(19) United States
(12) Patent Application Publication
(10) Pub. No.: US 2021/0246121 A1
(43) Pub. Date: Aug. 12, 2021

(54) SOLID FORMS OF APOL1 INHIBITOR AND METHODS OF USING SAME

(71) Applicant: Vertex Pharmaceuticals Incorporated, Boston, MA (US)

(72) Inventors: Mei-Hsiu LAI, Waltham, MA (US); Jicong LI, Cambridge, MA (US); Muna SHRESTHA, Belmont, MA (US); Ales MEDEK, Winchester, MA (US); Yi SHI, Natick, MA (US); Andrey PERESYPKIN, Waban, MA (US); Michael JUDELSON, Somerville, MA (US); Jack MINCHOM, Somerville, MA (US); Courtney MAGUIRE, Watertown, MA (US); Kevin GAGNON, Acton, MA (US); Faith WITKOS, Attleboro, MA (US); Satish Kumar IYEMPERUMAL, Quincy, MA (US); Kanika SARPAL, Waltham, MA (US)

(73) Assignee: Vertex Pharmaceuticals Incorporated

(21) Appl. No.: 17/166,927

(22) Filed: Feb. 3, 2021

Related U.S. Application Data
(60) Provisional application No. 63/038,267, filed on Jun. 12, 2020.
(60) Provisional application No. 62/970,002, filed on Feb. 4, 2020.

(51) Int. Cl. C07D 403/12

(52) U.S. Cl. CPC C07D 403/12; C07B 2200/13

(57) ABSTRACT

The disclosure provides novel solid state forms of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, compositions comprising the same, and methods of using the same, including use in treating APOL1 mediated kidney disease.

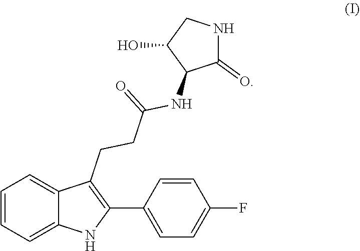 [SMILES: O=C(CCc1c(-c2ccc(F)cc2)[nH]c2ccccc12)N[C@@H]1C(=O)NC[C@H]1O]

This application claims priority to U.S. Provisional Patent Applications 62/970,002, filed Feb. 4, 2020 and 63/038,267, filed Jun. 12, 2020, the contents of which are incorporated by reference in their entirety. This disclosure provides solid forms of a compound that may inhibit apolipoprotein L1 (APOL1) and methods of using those compounds to treat APOL1 mediated kidney disease, including focal segmental glomerulosclerosis (FSGS) and/or non-diabetic kidney disease (NDKD). In some embodiments, the FSGS and/or NDKD is associated with common APOL1 genetic variants (G1: S342G:I384M and G2: N388del:Y389del).

FSGS is a disease of the podocyte (glomerular visceral epithelial cells) responsible for proteinuria and progressive decline in kidney function. NDKD is a disease characterized by hypertension and progressive decline in kidney function. Human genetics support a causal role for the G1 and G2 APOL1 variants in inducing kidney disease. Individuals with two APOL1 risk alleles are at increased risk of developing end-stage kidney disease (ESKD), including FSGS, human immunodeficiency virus (HIV)-associated nephropathy, NDKD, arterionephrosclerosis, lupus nephritis, microalbuminuria, and chronic kidney disease. See, P. Dummer et al., Semin Nephrol. 35(3): 222-236 (2015).

APOL1 is a 44 kDa protein that is only expressed in humans, gorillas, and baboons. APOL1 is produced mainly by the liver and contains a signal peptide that allows for secretion into the bloodstream, where it circulates bound to a subset of high density lipoproteins. APOL1 is responsible for protection against the invasive parasite, Trypanosoma brucei brucei (T. b. brucei). APOL1 G1 and G2 variants confer additional protection against trypanosoma species that cause sleeping sickness. Although normal plasma concentrations of APOL1 are relatively high and can vary at least 20-fold in humans, circulating APOL1 is not causally associated with kidney disease.

However, APOL1 in the kidney is thought to be responsible for the development kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry and in the U.S., ESKD is responsible for nearly as many lost years of life in women as from breast cancer and more lost years of life in men than from colorectal cancer. Currently, FSGS and NDKD are managed with symptomatic treatment (including blood pressure control using blockers of the renin angiotensin system), and patients with FSGS and heavy proteinuria may be offered high dose steroids. Corticosteroids induce remission in a minority of patients and are associated with numerous and at times, severe, side effects, and are often poorly tolerated. These patients, and particularly individuals of recent sub-Saharan African ancestry with two APOL1 risk alleles, experience faster disease progression leading to ESKD.

Thus, there is an unmet medical need for treatment for APOL1 mediated kidney diseases, including FSGS, NDKD, and ESKD. In view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Compound I, its method of preparation, physicochemical data are disclosed as Compound 87 in U.S. Provisional Application No. 62/780,667 filed on Dec. 17, 2018, the entirety of which is incorporated herein by reference. Additional information, such as solid state forms, are disclosed as Compound 87 in U.S. application Ser. No. 16/717,099 and PCT International Application No. PCT/US2019/066746, both of which were filed on Dec. 17, 2019, the entirety of each of which are incorporated herein by reference.

One aspect of the disclosure provides a new solid state Form B of Compound I, which can be employed in the treatment of diseases mediated by APOL1, such as FSGS and NDKD.

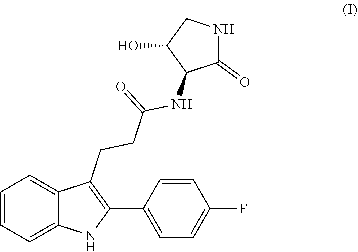 [SMILES: O=C(CCc1c(-c2ccc(F)cc2)[nH]c2ccccc12)N[C@@H]1C(=O)NC[C@H]1O]

Another aspect of the disclosure provides a new solid state form, citric acid cocrystal Form A, of Compound I, which can be employed in the treatment of diseases mediated by APOL1, such as FSGS and NDKD. Another aspect of the disclosure provides a new solid state form, piperazine cocrystal Form A, of Compound I, which can be employed in the treatment of diseases mediated by APOL1, such as FSGS and NDKD. Another aspect of the disclosure provides a new solid state form, urea cocrystal Form A, of Compound I, which can be employed in the treatment of diseases mediated by APOL1, such as FSGS and NDKD. Another aspect of the disclosure provides a new solid state form, nicotinamide cocrystal Form A, of Compound I, which can be employed in the treatment of diseases mediated by APOL1, such as FSGS and NDKD. Another aspect of the disclosure provides a new solid state form, nicotinamide cocrystal Form B, of Compound I, which can be employed in the treatment of diseases mediated by APOL1, such as FSGS and NDKD. Another aspect of the disclosure provides a new solid state form, aspartame cocrystal Form A, of Compound I, which can be employed in the treatment of diseases mediated by APOL1, such as FSGS and NDKD. Another aspect of the disclosure provides a new solid state form, glutaric acid cocrystal Form A, of Compound I, which can be employed in the treatment of diseases mediated by APOL1, such as FSGS and NDKD. Another aspect of the disclosure provides a new solid state form, L-proline cocrystal Form A, of Compound I, which can be employed in the treatment of diseases mediated by APOL1, such as FSGS and NDKD. Another aspect of the disclosure provides a new solid state form, L-proline cocrystal Form B, of Compound I, which can be employed in the treatment of diseases mediated by APOL1, such as FSGS and NDKD. Another aspect of the disclosure provides a new solid state form, vanillin cocrystal Form A, of Compound I, which can be employed in the treatment of diseases mediated by APOL1, such as FSGS and NDKD. Another aspect of the disclosure provides a new solid state form, 2-pyridone cocrystal Form A, of Compound I, which can be employed in the treatment of diseases mediated by APOL1, such as FSGS and NDKD.

Another aspect of the disclosure provides methods of treating FSGS and/or NDKD comprising administering to a subject in need thereof, one a solid form of Compound I selected from Compound I Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, or a pharmaceutical composition comprising the same.

In some embodiments, the methods of treatment include administration of at least one additional active agent to the subject in need thereof, either in the same pharmaceutical composition as a solid form Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, or as separate compositions.

Also provided are methods of inhibiting APOL1, comprising administering to a subject in need thereof, a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, or a pharmaceutical composition comprising the same.

### BRIEF DESCRIPTION OF THE DRAWINGS

FIG. 1A depicts an XRPD diffractogram of Compound I Form B—Lot 1.

FIG. 1B depicts an XRPD diffractogram of Compound I Form B—Lot 2. FIG. 1C provides a comparison of XRPD diffractograms of Compound I Form B Lot 1 (top line) and Lot 2 (bottom line). FIG. 1D shows the substantial similarity of XRPD diffractograms for six separate preparations of Compound I Form B.

FIG. 2 depicts a solid state ¹³C NMR spectrum of Form B of Compound I.

FIG. 3 depicts a ¹⁹F MAS spectrum of Form B of Compound I.

FIG. 4 depicts a TGA thermogram of Form B of Compound I.

FIG. 5 depicts a DSC curve of Form B of Compound I.

FIG. 6 depicts an IR spectrum of Form B of Compound I.

FIG. 7 depicts an XRPD diffractogram of citric acid cocrystal Form A of Compound I.

FIG. 8 depicts a solid state ¹³C NMR spectrum of citric acid cocrystal Form A of Compound I.

FIG. 9 depicts a ¹⁹F MAS spectrum of citric acid cocrystal Form A of Compound I.

FIG. 10 depicts a TGA thermogram of citric acid cocrystal Form A of Compound I.

FIG. 11 depicts a DSC curve of citric acid cocrystal Form A of Compound I.

FIG. 12 depicts an XRPD diffractogram of piperazine cocrystal Form A of Compound I.

FIG. 13 depicts a solid state ¹³C NMR spectrum of piperazine cocrystal Form A of Compound I.

FIG. 14 depicts a ¹⁹F MAS spectrum of piperazine cocrystal Form A of Compound I.

FIG. 15 depicts a TGA thermogram of piperazine cocrystal Form A of Compound I.

FIG. 16 depicts a DSC curve of piperazine cocrystal Form A of Compound I.

FIG. 17 depicts an XRPD diffractogram of urea cocrystal Form A of Compound I.

FIG. 18 depicts a solid state ¹³C NMR spectrum of urea cocrystal Form A of Compound I.

FIG. 19 depicts a ¹⁹F MAS spectrum of urea cocrystal Form A of Compound I.

FIG. 20 depicts a TGA thermogram of urea cocrystal Form A of Compound I.

FIG. 21 depicts a DSC curve of urea cocrystal Form A of Compound I.

FIG. 22 depicts an XRPD diffractogram of nicotinamide cocrystal Form A of Compound I.

FIG. 23 depicts a solid state ¹³C NMR spectrum of nicotinamide cocrystal Form A of Compound I.

FIG. 24 depicts a ¹⁹F MAS spectrum of nicotinamide cocrystal Form A of Compound I.

FIG. 25 depicts a TGA thermogram of nicotinamide cocrystal Form A of Compound I.

FIG. 26 depicts a DSC curve of nicotinamide cocrystal Form A of Compound I.

FIG. 27 depicts an XRPD diffractogram of nicotinamide cocrystal Form B of Compound I.

FIG. 28 depicts a solid state ¹³C NMR spectrum of nicotinamide cocrystal Form B of Compound I.

FIG. 29 depicts a ¹⁹F MAS spectrum of nicotinamide cocrystal Form B of Compound I.

FIG. 30 depicts an XRPD diffractogram of aspartame cocrystal Form A of Compound I.

FIG. 31 depicts a TGA thermogram of aspartame cocrystal Form A of Compound I.

FIG. 32 depicts a DSC curve of aspartame cocrystal Form A of Compound I.

FIG. 33 depicts an XRPD diffractogram of glutaric acid cocrystal Form A of Compound I.

FIG. 34 depicts a TGA thermogram of glutaric acid cocrystal Form A of Compound I.

FIG. 35 depicts a DSC curve of glutaric acid cocrystal Form A of Compound I.

FIG. 36 depicts an XRPD diffractogram of L-proline cocrystal Form A of Compound I.

FIG. 37 depicts a TGA thermogram of L-proline cocrystal Form A of Compound I.

FIG. 38 depicts a DSC curve of L-proline cocrystal Form A of Compound I.

FIG. 39A depicts an XRPD diffractogram of L-proline cocrystal Form B of Compound I. FIG. 39B depicts a solid state ¹³C NMR spectrum of L-proline cocrystal Form B of Compound I. FIG. 39C depicts a ¹⁹F MAS spectrum of L-proline cocrystal Form B of Compound I.

FIG. 40 depicts a TGA thermogram of L-proline cocrystal Form B of Compound I.

FIG. 41 depicts a DSC curve of L-proline cocrystal Form B of Compound I.

FIG. 42A provides an XRPD diffractogram of vanillin cocrystal Form A of Compound I. FIG. 42B depicts a solid state ¹³C NMR spectrum of vanillin cocrystal Form A of Compound I. FIG. 42C depicts a ¹⁹F MAS spectrum of vanillin cocrystal Form A of Compound I.

FIG. 43 a TGA thermogram of vanillin cocrystal Form A of Compound I.

FIG. 44 a DSC curve of vanillin cocrystal Form A of Compound I.

FIG. 45 depicts an XRPD diffractogram of 2-pyridone cocrystal Form A of Compound I.

FIG. 46A depicts a solid state ¹³C NMR full spectrum ¹³C CPMAS of 2-pyridone cocrystal Form A of Compound I. FIG. 46B depicts a solid state ¹³C NMR spectrum of 2-pyridone cocrystal Form A of Compound I after subtraction of Compound I Form B and amorphous Compound I.

FIG. 47A depicts a full spectrum ¹⁹F MAS of 2-pyridone cocrystal Form A of Compound I. FIG. 47B depicts a ¹⁹F MAS spectrum of 2-pyridone cocrystal Form A of Compound I after subtraction of Compound I Form B and amorphous Compound I.

FIG. 48 depicts a TGA thermogram of 2-pyridone cocrystal Form A of Compound I.

FIG. 49 depicts a DSC curve of 2-pyridone cocrystal Form A of Compound I.

FIG. 50 depicts an XRPD diffractogram of Compound I Form A.

FIG. 51 depicts a solid state ¹³C NMR spectrum for Compound I Form A.

FIG. 52 depicts a ¹⁹F MAS (magnetic angle spinning) spectrum for Compound I Form A.

### DEFINITIONS

The term “APOL1” as used herein means apolipoprotein L1 protein and the term “APOL1” means apolipoprotein L1 gene.

The term “APOL1 mediated kidney disease” refers to a disease or condition that impairs kidney function and can be attributed to APOL1. In some embodiments APOL1 mediated kidney disease is associated with patients having two APOL1 risk alleles, e.g., are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease.

The term “FSGS” as used herein means focal segmental glomerulosclerosis, which is a disease of the podocyte (glomerular visceral epithelial cells) responsible for proteinuria and progressive decline in kidney function. In some embodiments FSGS is associated with two APOL1 risk alleles.

The term “NDKD” as used herein means non-diabetic kidney disease, which is characterized by severe hypertension and progressive decline in kidney function. In some embodiments, NDKD is associated with two APOL1 risk alleles.

The terms “ESKD” and “ESRD” are used interchangeabley to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

Non-limiting, examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting, examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The terms “about” and “approximately”, when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent. In some embodiments, the term “about” refers to a value ±10%, ±8%, ±6%, ±5%, ±4%, ±2%, or ±1% of a referenced value.

The terms “patient” and “subject” are used interchangeably and refer to an animal including a human.

The terms “effective dose” and “effective amount” are used interchangeably herein and refer to that amount of compound that produces the desired effect for which it is administered (e.g., improvement in symptoms of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to the following: complete or partial remission, lower risk of kidney failure (e.g. ESRD), and disease-related complications (e.g. edema, susceptibility to infections, or thrombo-embolic events). Improvements in or lessening the severity of any of these symptoms can be readily assessed according to methods and techniques known in the art or subsequently developed. In some embodiments, the terms “treat,” “treating,” and “treatment,” refer to the lessening of severity of one or more symptoms of FSGS and/or NDKD.

The solid forms of Compound I disclosed herein may be administered once daily, twice daily, or three times daily, for example, for the treatment of FSGS. In some embodiments, the solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is administered once daily. In some embodiments, the solid form of Compound I is administered twice daily. In some embodiments, the solid form of Compound I is administered three times daily.

In some embodiments, 2 mg to 1500 mg of the solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is administered once daily, twice daily, or three times daily.

As used herein, the term “ambient conditions” means room temperature, open air condition and uncontrolled humidity condition.

As used herein, the terms “crystalline form” and “Form” interchangeably refer to a crystal structure (or polymorph) having a particular molecular packing arrangement in the crystal lattice. Crystalline forms can be identified and distinguished from each other by one or more characterization techniques including, for example, X-ray powder diffraction (XRPD), single crystal X-ray diffraction, solid state nuclear magnetic resonance (SSNMR), differential scanning calorimetry (DSC), infrared radiation (IR), and/or thermogravimetric analysis (TGA). Accordingly, as used herein, the terms “crystalline Form B of Compound I” refers to a unique crystalline form that can be identified and distinguished from other crystalline forms of Compound I by one or more characterization techniques including, for example, X-ray powder diffraction (XRPD), single crystal X-ray diffraction, SSNMR, differential scanning calorimetry (DSC), infrared radiation (IR), and/or thermogravimetric analysis (TGA). In some embodiments, the novel crystalline Form B of is characterized by an X-ray powder diffractogram having one or more signals at one or more specified two-theta values (° 2θ).

As used herein, the term “SSNMR” refers to the analytical characterization method of solid state nuclear magnetic resonance. SSNMR spectra can be recorded at ambient conditions on any magnetically active isotope present in the sample. The typical examples of active isotopes for small molecule active pharmaceutical ingredients include ¹H, ²H, ¹³C, ¹⁹F, ³¹P, ¹⁵N, ¹⁴N, ³⁵Cl, ¹¹B, ⁷Li, ¹⁷O, ²³Na, ⁷⁹Br, and ¹⁹⁵Pt.

As used herein, the term “XRPD” refers to the analytical characterization method of X-ray powder diffraction. XRPD patterns can be recorded at ambient conditions in transmission or reflection geometry using a diffractometer.

As used herein, the terms “X-ray powder diffractogram,” “X-ray powder diffraction pattern,” “XRPD pattern” interchangeably refer to an experimentally obtained pattern plotting signal positions (on the abscissa) versus signal intensities on the ordinate). For an amorphous material, an X-ray powder diffractogram may include one or more broad signals; and for a crystalline material, an X-ray powder diffractogram may include one or more signals, each identified by its angular value as measured in degrees 2θ (°2θ), depicted on the abscissa of an X-ray powder diffractogram, which may be expressed as “a signal at . . . degrees two-theta,” “a signal at [a] two-theta value(s) of . . . ” and/or “a signal at at least . . . two-theta value(s) chosen from . . . .”

A “signal” or “peak” as used herein refers to a point in the XRPD pattern where the intensity as measured in counts is at a local maximum. One of ordinary skill in the art would recognize that one or more signals (or peaks) in an XRPD pattern may overlap and may, for example, not be apparent to the naked eye. Indeed, one of ordinary skill in the art would recognize that some art-recognized methods are capable of and suitable for determining whether a signal exists in a pattern, such as Rietveld refinement.

As used herein, “a signal at . . . degrees two-theta,” “a signal at [a] two-theta value[ ] of . . . ” and/or “a signal at at least . . . two-theta value(s) chosen from . . . ” refer to X-ray reflection positions as measured and observed in X-ray powder diffraction experiments (°2θ).

The repeatability of the angular values is in the range of ±0.2° 2θ, i.e., the angular value can be at the recited angular value+0.2 degrees two-theta, the angular value −0.2 degrees two-theta, or any value between those two end points (angular value +0.2 degrees two-theta and angular value −0.2 degrees two-theta).

The terms “signal intensities” and “peak intensities” interchangeably refer to relative signal intensities within a given X-ray powder diffractogram. Factors that can affect the relative signal or peak intensities include sample thickness and preferred orientation (e.g., the crystalline particles are not distributed randomly).

The term “X-ray powder diffractogram having a signal at . . . two-theta values” as used herein refers to an XRPD pattern that contains X-ray reflection positions as measured and observed in X-ray powder diffraction experiments (°2θ).

As used herein, an X-ray powder diffractogram is “substantially similar to that in [a particular] Figure” when at least 90%, such as at least 95%, at least 98%, or at least 99%, of the signals in the two diffractograms overlap. In determining “substantial similarity,” one of ordinary skill in the art will understand that there may be variation in the intensities and/or signal positions in XRPD diffractograms even for the same crystalline form. Thus, those of ordinary skill in the art will understand that the signal positions in XRPD diffractograms (in degrees two-theta (°2θ) referred to herein) generally mean that value reported is 0.2 degrees 2θ of the reported value, an art-recognized variance.

As used herein, an ssNMR spectrum is “substantially similar to that in [a particular] Figure” when at least 90%, such as at least 95%, at least 98%, or at least 99%, of the signals in the two spectra overlap. In determining “substantial similarity,” one of ordinary skill in the art will understand that there may be variation in the intensities and/or signal positions in SSNMR spectra even for the same crystalline form. Thus, those of ordinary skill in the art will understand that the signal positions in ssNMR spectra (in ppm) referred to herein generally mean that value reported is 0.2 ppm of the reported value, an art-recognized variance.

As used herein, a crystalline form is “substantially pure” when it accounts for an amount by weight equal to or greater than 90% of the sum of all solid form(s) in a sample as determined by a method in accordance with the art, such as quantitative XRPD. In some embodiments, the solid form is “substantially pure” when it accounts for an amount by weight equal to or greater than 95% of the sum of all solid form(s) in a sample. In some embodiments, the solid form is “substantially pure” when it accounts for an amount by weight equal to or greater than 99% of the sum of all solid form(s) in a sample.

As used herein, the term “DSC” refers to the analytical method of Differential Scanning Calorimetry.

As used herein, the term “TGA” refers to the analytical method of Thermo Gravimetric (or thermogravimetric) Analysis.

Compound I is disclosed as Compound 87 in U.S. Provisional Application No. 62/780,667 filed on Dec. 17, 2018, U.S. application Ser. No. 16/717,099 filed on Dec. 17, 2019, and PCT International Application No. PCT/US2019/066746 filed on Dec. 17, 2019, the entire contents of each of which are incorporated herein by reference.

Compound I is depicted as follows:

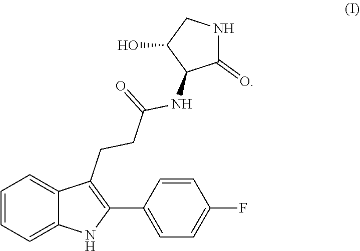 [SMILES: O=C(CCc1c(-c2ccc(F)cc2)[nH]c2ccccc12)N[C@@H]1C(=O)NC[C@H]1O]

Forms of Compound I as Form A, Hydrate Form A, IPAc Solvate, and Amorphous Form of Compound I, are disclosed in U.S. application Ser. No. 16/717,099 and PCT International Application No. PCT/US2019/066746, both of which were filed on Dec. 17, 2019 and both of which are incorporated herein by reference.

### Compound I Form B

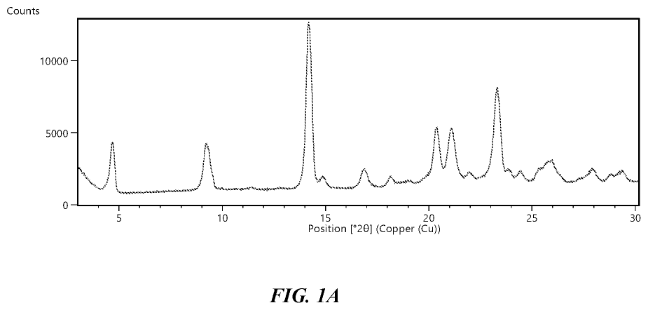

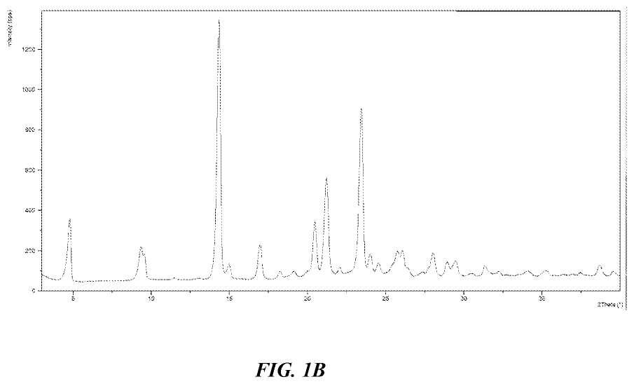

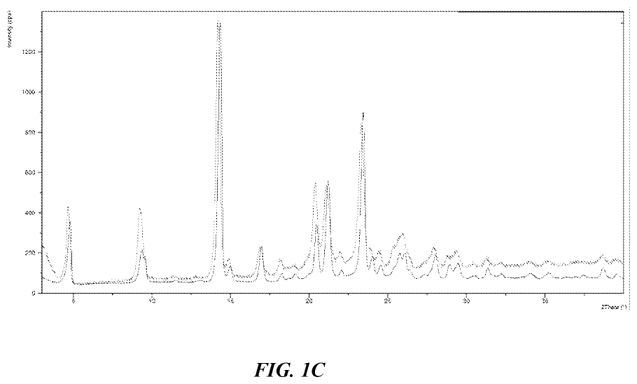

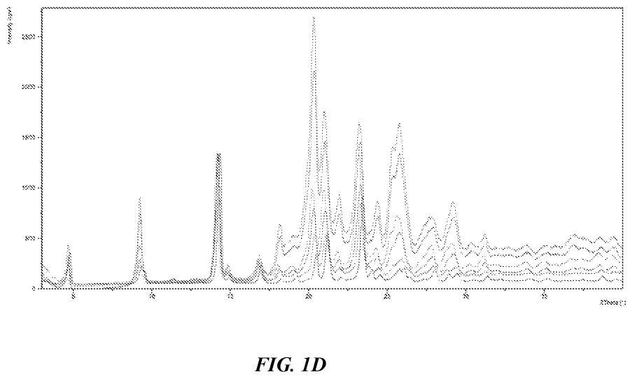

One embodiment of the invention provides novel Form B of Compound I. In some embodiments, Form B of Compound I is substantially pure. In some embodiments, Form B is characterized by an X-ray powder diffractogram substantially similar to that in FIG. 1A. In some embodiments, Form B of Compound I is characterized by an X-ray powder diffractogram having a signal at 20.3±0.2 two-theta. In some embodiments, Form B of Compound I is characterized by an X-ray powder diffractogram having a signal at 20.3±0.2 and a signal at one or more two-theta values chosen from 4.7±0.2, 9.2±0.2, 14.2±0.2, 21.1±0.2, and 23.3±0.2. In some embodiments, Form B of Compound I is characterized by an X-ray powder diffractogram having a signal at least two two-theta values chosen from 14.2±0.2, 20.3±0.2, 21.1±0.2, and 23.3±0.2. In some embodiments, Form B of Compound I is characterized by an X-ray powder diffractogram having a signal at at least three two-theta values chosen from 14.2±0.2, 20.3±0.2, 21.1±0.2, and 23.3±0.2. In some embodiments, Form B of Compound I is characterized by an X-ray powder diffractogram having a signal at the following two-theta values 14.2±0.2, 20.3±0.2, 21.1±0.2, and 23.3±0.2.

In some embodiments, Form B of Compound I is characterized by an X-ray powder diffractogram having a signal at at least two two-theta values chosen from 4.7±0.2, 9.2±0.2, 14.2±0.2, 20.3±0.2, 21.1±0.2, and 23.3±0.2. In some embodiments, Form B of Compound I is characterized by an X-ray powder diffractogram having a signal at at least three two-theta values chosen from 4.7±0.2, 9.2±0.2, 14.2±0.2, 20.3±0.2, 21.1±0.2, and 23.3±0.2. In some embodiments, Form B of Compound I is characterized by an X-ray powder diffractogram having a signal at at least four two-theta values chosen from 4.7±0.2, 9.2±0.2, 14.2±0.2, 20.3±0.2, 21.1±0.2, and 23.3±0.2. In some embodiments, Form B of Compound I is characterized by an X-ray powder diffractogram having a signal at at least five two-theta values chosen from 4.7±0.2, 9.2±0.2, 14.2±0.2, 20.3±0.2, 21.1±0.2, and 23.3±0.2. In some embodiments, Form B of Compound I is characterized by an X-ray powder diffractogram having a signal at the following two-theta values 4.7±0.2, 9.2±0.2, 14.2±0.2, 20.3±0.2, 21.1±0.2, and 23.3±0.2.

In alternate embodiments, Form B of Compound I is characterized by an X-ray powder diffractogram substantially similar to that in FIG. 1B. In some embodiments, Form B of Compound I is characterized by an X-ray powder diffractogram having a signal at one or more two-theta values chosen from 16.9±0.2, 20.4±0.2, and 23.4±0.2. In some embodiments, Form B of Compound I is characterized by an X-ray powder diffractogram having a signal at two or more two-theta values chosen from 16.9±0.2, 20.4±0.2, and 23.4±0.2. In some embodiments, Form B of Compound I is characterized by an X-ray powder diffractogram having a signal at two-theta values 16.9±0.2, 20.4±0.2, and 23.4±0.2.

In some embodiments, Form B of Compound I is characterized by an X-ray powder diffractogram having a signal at (a) one or more two-theta values chosen from 16.9±0.2, 20.4±0.2, and 23.4±0.2; and (b) one, two, or three, two-theta values chosen from 4.7±0.2, 9.3±0.2, 9.6±0.2, 14.3±0.2, and 21.2±0.2. In some embodiments, Form B of Compound I is characterized by an X-ray powder diffractogram having a signal at (a) two or more two-theta values chosen from 16.9±0.2, 20.4±0.2, and 23.4±0.2; and (b) at two-theta values of 4.7±0.2, 9.3±0.2, 9.6±0.2, 14.3±0.2, and 21.2±0.2. In some embodiments, Form B of Compound I is characterized by an X-ray powder diffractogram having a signal at two-theta values of 4.7±0.2, 9.3±0.2, 9.6±0.2, 14.3±0.2, 16.9±0.2, 20.4±0.2, 21.2±0.2 and 23.4±0.2.

In some embodiments, disclosed herein is a composition comprising Form B of Compound I. In some embodiments, disclosed herein is a composition comprising Compound I in substantially pure Form B. In some embodiments, disclosed herein is a composition comprising at least one active compound consisting essentially of Compound I in Form B.

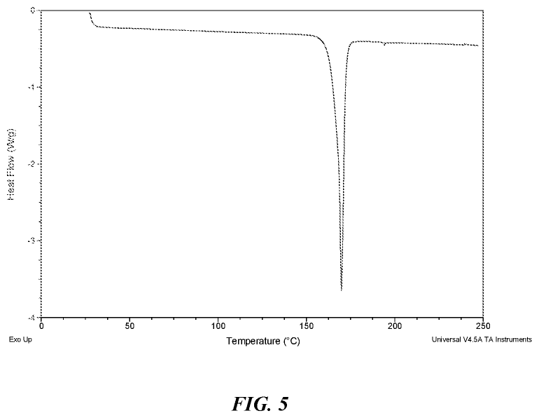

In some embodiments, Form B of Compound I is characterized by a DSC substantially similar to that in FIG. 5. In some embodiments, Form B of Compound I is characterized by a DSC having a melting onset of 168° C. with a peak at 170° C. In some embodiments, Form B of Compound I is characterized by a DSC having a peak in a range of 167° C. to 171° C.

In some embodiments, Form B of Compound I is characterized by a ¹³C NMR spectrum having a signal at at least one, at least two, at least three, at least four, or at least five ppm value(s) chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm. In some embodiments, Form B of Compound I is characterized by a ¹³C NMR spectrum having a signal at at least seven, at least ten, at least twelve, or at least fifteen ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.

In some embodiments, Form B of Compound I is characterized by a ¹³C NMR spectrum having a signal at at least one ppm value chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least two ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least three ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least four ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least five ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B is characterized by a ¹³C NMR spectrum having a signal at at least six ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least seven ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least eight ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at at least nine ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm. In some embodiments, Form B of Compound I is characterized by a ¹³C NMR spectrum having a signal at 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.

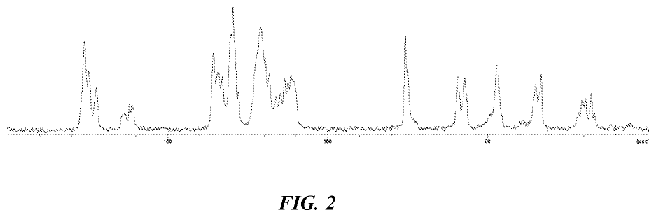

In some embodiments, Form B is characterized by a ¹³C NMR spectrum substantially similar to that in FIG. 2.

In some embodiments, Form B of Compound I is characterized by a ¹⁹F NMR spectrum having a signal at −112.5±0.2 ppm. In some embodiments, Form B of Compound I is characterized by a ¹⁹F NMR spectrum having signals at at least two ppm values chosen from −109.4±0.2 ppm, −112.5±0.2 ppm, and −113.7±0.2 ppm. In some embodiments, Form B of Compound I is characterized by a ¹⁹F NMR spectrum having signals at −109.4±0.2 ppm, −112.5±0.2 ppm, and −113.7±0.2 ppm.

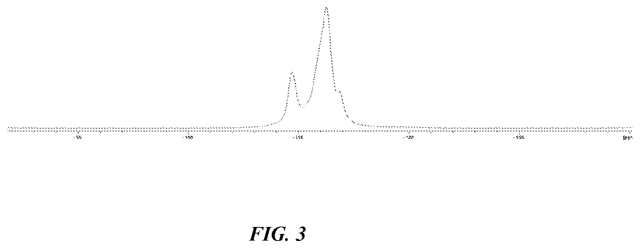

In some embodiments, Form B is characterized by a ¹⁹F NMR spectrum substantially similar to that in FIG. 3.

### Citric Acid Cocrystal Form A of Compound I

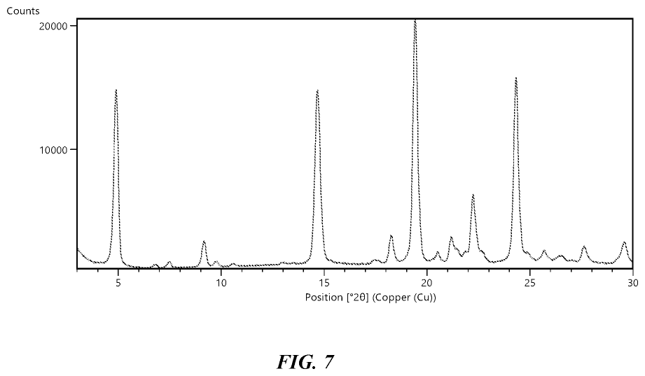

One embodiment of the invention provides a citric acid cocrystal Form A of Compound I. In some embodiments, the citric acid cocrystal Form A of Compound I is substantially pure. In some embodiments, the citric acid cocrystal Form A is characterized by an X-ray powder diffractogram substantially similar to that in FIG. 7. In some embodiments, the citric acid cocrystal Form A of Compound I is characterized by an X-ray powder diffractogram having a signal at one or more two-theta values selected from 24.4±0.2 19.5±0.2, 14.6±0.2, and 4.9±0.2. In some embodiments, the citric acid cocrystal Form A of Compound I is characterized by an X-ray powder diffractogram having a signal at the following two-theta values 24.4±0.2 19.5±0.2, 14.6±0.2, and 4.9±0.2. In some embodiments, the citric acid cocrystal Form A of Compound I is characterized by an X-ray powder diffractogram having (a) a signal at the following two-theta values 24.4±0.2 19.5±0.2, 14.6±0.2, and 4.9±0.2; and (b) a signal at one or more two-theta values selected from 22.2±0.2, 21.2±0.2, 18.3±0.2, 18.2±0.2, and 9.2±0.2. In some embodiments, the citric acid cocrystal Form A of Compound I is characterized by an X-ray powder diffractogram having signals at the following two-theta values 24.4±0.2, 22.2±0.2, 21.2±0.2, 19.5±0.2, 18.3±0.2, 18.2±0.2, 14.6±0.2, 9.2±0.2, and 4.9±0.2.

In some embodiments, the citric acid cocrystal Form A of Compound I is characterized by a ¹³C NMR spectrum having one or more signals selected from 174.8±0.2 ppm, 173.8±0.2 ppm, 130.1±0.2 ppm, 74.8±0.2 ppm, and 71.8±0.2 ppm. In some embodiments, the citric acid cocrystal Form A of Compound I is characterized by a 13C NMR spectrum having signals at 174.8±0.2 ppm, 173.8±0.2 ppm, 130.1±0.2 ppm, 74.8±0.2 ppm, and 71.8±0.2 ppm. In some embodiments, the citric acid cocrystal Form A of Compound I is characterized by a ¹³C NMR spectrum having (a) signals at 174.8±0.2 ppm, 173.8±0.2 ppm, 130.1±0.2 ppm, 74.8±0.2 ppm, and 71.8±0.2 ppm; and (b) one or more signals selected from 179.9±0.2 ppm, 129.4±0.2 ppm, 122.4±0.2 ppm, 116.3±0.2 ppm, and 44.1±0.2 ppm. In some embodiments, the citric acid cocrystal Form A of Compound I is characterized by a ¹³C NMR spectrum having signals at 179.9±0.2 ppm, 174.8±0.2 ppm, 173.8±0.2 ppm, 130.1±0.2 ppm, 129.4±0.2 ppm, 122.4±0.2 ppm, 116.3±0.2 ppm, 74.8±0.2 ppm, 71.8±0.2 and 44.1±0.2 ppm.

In some embodiments, the citric acid cocrystal Form A of Compound I is characterized by a ¹⁹F NMR spectrum having a signal at one or more ppm values chosen from −112.6±0.2 ppm, −114.8±0.2 ppm, and −116.8±0.2 ppm. In some embodiments, the citric acid cocrystal Form A of Compound I is characterized by a ¹⁹F NMR spectrum having signals at 112.6±0.2 ppm, −114.8±0.2 ppm, and −116.8±0.2 ppm.

### Piperazine Cocrystal Form A of Compound I

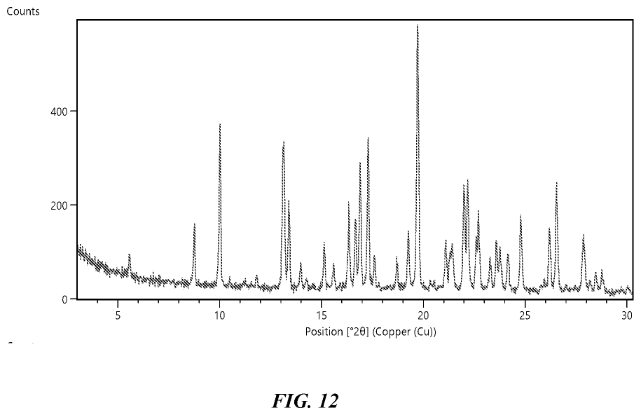

One embodiment of the invention provides a piperazine cocrystal Form A of Compound I. In some embodiments, the piperazine cocrystal Form A of Compound I is substantially pure. In some embodiments, the piperazine cocrystal Form A is characterized by an X-ray powder diffractogram substantially similar to that in FIG. 12. In some embodiments, the piperazine cocrystal Form A of Compound I is characterized by an X-ray powder diffractogram having a signal at one or more two-theta values selected from 19.7±0.2, 17.3±0.2, 13.1±0.2, and 10.0±0.2. In some embodiments, the piperazine cocrystal Form A of Compound I is characterized by an X-ray powder diffractogram having a signal at the following two-theta values 19.7±0.2, 17.3±0.2, 13.1±0.2, and 10.0±0.2. In some embodiments, the piperazine cocrystal Form A of Compound I is characterized by an X-ray powder diffractogram having (a) a signal at the following two-theta values 19.7±0.2, 17.3±0.2, 13.1±0.2, and 10.0±0.2; and (b) a signal at one or more two-theta values selected from 26.5±0.2, 22.2±0.2, 22.0±0.2, 16.9±0.2, 16.3±0.2, and 13.4±0.2. In some embodiments, the piperazine cocrystal Form A of Compound I is characterized by an X-ray powder diffractogram having signals at the following two-theta values 26.5±0.2, 22.2±0.2, 22.0±0.2, 19.7±0.2, 17.3±0.2, 16.9±0.2, 16.3±0.2, 13.4±0.2, 13.1±0.2, and 10.0±0.2.

In some embodiments, the piperazine cocrystal Form A of Compound I is characterized by a ¹³C NMR spectrum having one or more signals selected from 111.0±0.2 ppm, 72.8±0.2 ppm, 47.0±0.2 ppm, 45.1±0.2 ppm, and 44.8±0.2 ppm. In some embodiments, the piperazine cocrystal Form A of Compound I is characterized by a 13C NMR spectrum having signals at 111.0±0.2 ppm, 72.8±0.2 ppm, 47.0±0.2 ppm, 45.1±0.2 ppm, and 44.8±0.2 ppm. In some embodiments, the piperazine cocrystal Form A of Compound I is characterized by a ¹³C NMR spectrum having (a) signals at 111.0±0.2 ppm, 72.8±0.2 ppm, 47.0±0.2 ppm, 45.1±0.2 ppm, and 44.8±0.2 ppm and (b) one or more signals selected from 130.5±0.2 ppm, 129.2±0.2 ppm, 129.0±0.2 ppm, 120.5±0.2 ppm, 119.9±0.2 ppm, 111.6±0.2 ppm, and 46.2±0.2 ppm. In some embodiments, the piperazine cocrystal Form A of Compound I is characterized by a ¹³C NMR spectrum having signals at 130.5±0.2 ppm, 129.2±0.2 ppm, 120.5±0.2 ppm, 119.9±0.2 ppm, 111.6±0.2 ppm, 111.0±0.2 ppm, 72.8±0.2 ppm, 47.0±0.2 ppm, 46.2±0.2 ppm 45.1±0.2 ppm, and 44.8±0.2 ppm.

In some embodiments, the piperazine cocrystal Form A of Compound I is characterized by a ¹⁹F NMR spectrum having a signal at −112.1±0.2 ppm.

### Urea Cocrystal Form A of Compound I

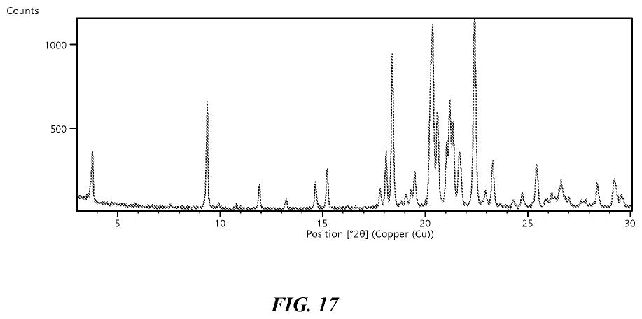

One embodiment of the invention provides a urea cocrystal Form A of Compound I. In some embodiments, the urea cocrystal Form A of Compound I is substantially pure. In some embodiments, the urea cocrystal Form A is characterized by an X-ray powder diffractogram substantially similar to that in FIG. 17. In some embodiments, the urea cocrystal Form A of Compound I is characterized by an X-ray powder diffractogram having a signal at one or more two-theta values selected from 22.4±0.2, 21.2±0.2, 20.4±0.2, and 18.4±0.2. In some embodiments, the urea cocrystal Form A of Compound I is characterized by an X-ray powder diffractogram having a signal at the following two-theta values 22.4±0.2, 21.2±0.2, 20.4±0.2, and 18.4±0.2. In some embodiments, the urea cocrystal Form A of Compound I is characterized by an X-ray powder diffractogram having (a) a signal at the following two-theta values 22.4±0.2, 21.2±0.2, 20.4±0.2, and 18.4±0.2; and (b) a signal at one or more two-theta values selected from 23.3±0.2, 21.7±0.2, 21.4±0.2, 21.3±0.2, 20.3±0.2, and 9.4±0.2. In some embodiments, the urea cocrystal Form A of Compound I is characterized by an X-ray powder diffractogram having signals at the following two-theta values 23.3±0.2, 22.4±0.2, 21.7±0.2, 21.4±0.2, 21.3±0.2, 21.2±0.2, 20.4±0.2, 20.3±0.2, 18.4±0.2, and 9.4±0.2.

In some embodiments, the urea cocrystal Form A of Compound I is characterized by a ¹³C NMR spectrum having one or more signals selected from 129.2±0.2 ppm, 120.3±0.2 ppm, 74.6±0.2 ppm, 58.4±0.2 ppm, and 44.6±0.2 ppm. In some embodiments, the urea cocrystal Form A of Compound I is characterized by a ¹³C NMR spectrum having signals at 129.2±0.2 ppm, 120.3±0.2 ppm, 74.6±0.2 ppm, 58.4±0.2 ppm, and 44.6±0.2 ppm. In some embodiments, the urea cocrystal Form A of Compound I is characterized by a ¹³C NMR spectrum having (a) signals at 129.2±0.2 ppm, 120.3±0.2 ppm, 74.6±0.2 ppm, 58.4±0.2 ppm, and 44.6±0.2 ppm; and (b) one or more signals selected from 175.4±0.2 ppm, 175.0±0.2 ppm, 135.5±0.2 ppm, 38.4±0.2 ppm, and 18.9±0.2 ppm. In some embodiments, the urea cocrystal Form A of Compound I is characterized by a ¹³C NMR spectrum having signals at 175.4±0.2 ppm, 175.0±0.2 ppm, 135.5±0.2 ppm, 129.2±0.2 ppm, 120.3±0.2 ppm, 74.6±0.2 ppm, 58.4±0.2 ppm, and 44.6±0.2 ppm, 38.4±0.2 ppm, and 18.9±0.2 ppm.

In some embodiments, the urea cocrystal Form A of Compound I is characterized by a ¹⁹F NMR spectrum having a signal at one or more of −110.8±0.2 ppm, −113.2±0.2 ppm, and −113.7±0.2 ppm. In some embodiments, the urea cocrystal Form A of Compound I is characterized by a ¹⁹F NMR spectrum having signals at −110.8±0.2 ppm, −113.2±0.2 ppm, and −113.7±0.2 ppm.

### Nicotinamide Cocrystal Form A of Compound I

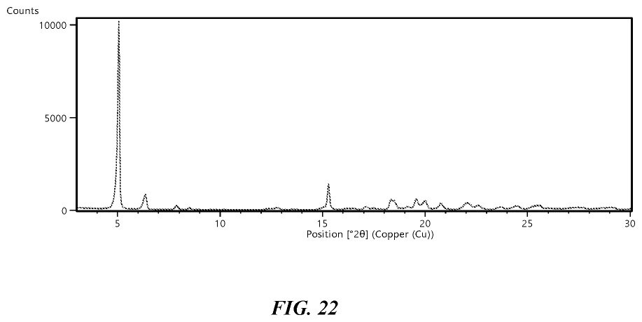

One embodiment of the invention provides a nicotinamide cocrystal Form A of Compound I. In some embodiments, the nicotinamide cocrystal Form A of Compound I is substantially pure. In some embodiments, the nicotinamide cocrystal Form A is characterized by an X-ray powder diffractogram substantially similar to that in FIG. 22. In some embodiments, the nicotinamide cocrystal Form A of Compound I is characterized by an X-ray powder diffractogram having a signal at one or more two-theta values selected from 18.3±0.2, 15.3±0.2, 6.3±0.2, and 5.1±0.2. In some embodiments, the nicotinamide cocrystal Form A of Compound I is characterized by an X-ray powder diffractogram having a signal at the following two-theta values 18.3±0.2, 15.3±0.2, 6.3±0.2, and 5.1±0.2. In some embodiments, the nicotinamide cocrystal Form A of Compound I is characterized by an X-ray powder diffractogram having (a) a signal at one or more two-theta values selected from 18.3±0.2, 15.3±0.2, 6.3±0.2, and 5.1±0.2; and (b) a signal at 19.6±0.2 degrees two-theta. In some embodiments, the nicotinamide cocrystal Form A of Compound I is characterized by an X-ray powder diffractogram having signals at the following two-theta values 19.6±0.2, 18.3±0.2, 15.3±0.2, 6.3±0.2, and 5.1±0.2.

In some embodiments, the nicotinamide cocrystal Form A of Compound I is characterized by a ¹³C NMR spectrum having one or more signals selected from 149.2±0.2 ppm, 136.1±0.2 ppm, 128.3±0.2 ppm, 112.0±0.2 ppm, and 71.4±0.2 ppm. In some embodiments, the nicotinamide cocrystal Form A of Compound I is characterized by a ¹³C NMR spectrum having signals at 149.2±0.2 ppm, 136.1±0.2 ppm, 128.3±0.2 ppm, 112.0±0.2 ppm, and 71.4±0.2 ppm. In some embodiments, the nicotinamide cocrystal Form A of Compound I is characterized by a ¹³C NMR spectrum having (a) signals at 149.2±0.2 ppm, 136.1±0.2 ppm, 128.3±0.2 ppm, 112.0±0.2 ppm, and 71.4±0.2 ppm; and (b) one or more signals selected from 174.5±0.2 ppm, 129.0±0.2 ppm, 121.2±0.2 ppm, 119.2±0.2 ppm, and 112.7±0.2 ppm. In some embodiments, the nicotinamide cocrystal Form A of Compound I is characterized by a ¹³C NMR spectrum having signals at 174.5±0.2 ppm, 149.2±0.2 ppm, 136.1±0.2 ppm, 129.0±0.2 ppm, 128.3±0.2 ppm, 121.2±0.2 ppm, 119.2±0.2 ppm, 112.7±0.2 ppm, 112.0±0.2 ppm, and 71.4±0.2 ppm.

In some embodiments, the nicotinamide cocrystal Form A of Compound I is characterized by a ¹⁹F NMR spectrum having a signal at one or more of −116.4±0.2 ppm, −117.9±0.2 ppm, and −118.5±0.2 ppm. In some embodiments, the nicotinamide cocrystal Form A of Compound I is characterized by a ¹⁹F NMR spectrum having signals at −116.4±0.2 ppm, −117.9±0.2 ppm, and −118.5±0.2 ppm.

### Nicotinamide Cocrystal Form B of Compound I

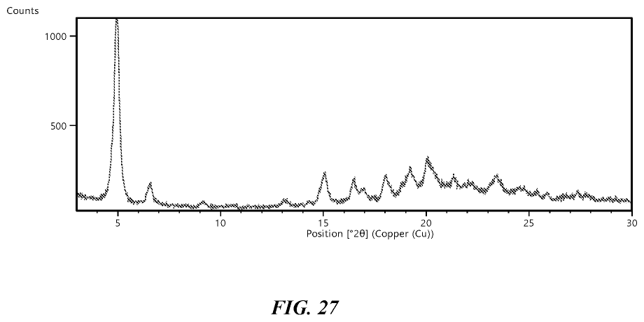

One embodiment of the invention provides a nicotinamide cocrystal Form B of Compound I. In some embodiments, the nicotinamide cocrystal Form B of Compound I is substantially pure. In some embodiments, the nicotinamide cocrystal Form B is characterized by an X-ray powder diffractogram substantially similar to that in FIG. 27. In some embodiments, the nicotinamide cocrystal Form B of Compound I is characterized by an X-ray powder diffractogram having a signal at one or more two-theta values selected from 20.0±0.2, 15.1±0.2, 5.0±0.2, and 4.9±0.2. In some embodiments, the nicotinamide cocrystal Form B of Compound I is characterized by an X-ray powder diffractogram having a signal at the following two-theta values 20.0±0.2, 15.1±0.2, 5.0±0.2, and 4.9±0.2. In some embodiments, the nicotinamide cocrystal Form B of Compound I is characterized by an X-ray powder diffractogram having (a) a signal at the following two-theta values 20.0±0.2, 15.1±0.2, 5.0±0.2, and 4.9±0.2; and (b) a signal at one or more of the following two-theta values selected from 19.2±0.2, 18.0±0.2, 16.5±0.2, and 6.6±0.2. In some embodiments, the nicotinamide cocrystal Form B of Compound I is characterized by an X-ray powder diffractogram having signals at the following two-theta values 20.0±0.2, 19.2±0.2, 18.0±0.2, 16.5±0.2, 15.1±0.2, 6.6±0.2, 5.0±0.2, and 4.9±0.2.

In some embodiments, the nicotinamide cocrystal Form B of Compound I is characterized by a ¹³C NMR spectrum having one or more signals selected from 136.4±0.2 ppm, 128.9±0.2 ppm, 121.7±0.2 ppm, 119.2±0.2 ppm, and 111.6±0.2 ppm. In some embodiments, the nicotinamide cocrystal Form B of Compound I is characterized by a ¹³C NMR spectrum having signals at 136.4±0.2 ppm, 128.9±0.2 ppm, 121.7±0.2 ppm, 119.2±0.2 ppm, and 111.6±0.2 ppm. In some embodiments, the nicotinamide cocrystal Form B of Compound I is characterized by a ¹³C NMR spectrum having (a) signals at 136.4±0.2 ppm, 128.9±0.2 ppm, 121.7±0.2 ppm, 119.2±0.2 ppm, and 111.6±0.2 ppm; and (b) one or more signals selected from 174.5±0.2 ppm, 120.6±0.2 ppm, 120.2±0.2 ppm, 62.8±0.2 ppm, and 18.1±0.2 ppm. In some embodiments, the nicotinamide cocrystal Form B of Compound I is characterized by a ¹³C NMR spectrum having signals at 174.5±0.2 ppm, 136.4±0.2 ppm, 128.9±0.2 ppm, 121.7±0.2 ppm, 120.6±0.2 ppm, 120.2±0.2 ppm, 119.2±0.2 ppm, 111.6±0.2 ppm 62.8±0.2 ppm, and 18.1±0.2 ppm.

In some embodiments, the nicotinamide cocrystal Form B of Compound I is characterized by a ¹⁹F NMR spectrum having a signal at one or more of −111.0±0.2 ppm, −113.0±0.2 ppm, and −115.4±0.2 ppm. In some embodiments, the nicotinamide cocrystal Form B of Compound I is characterized by a ¹⁹F NMR spectrum having signals at −111.0±0.2 ppm, −113.0±0.2 ppm, and −115.4±0.2 ppm.

### Aspartame Cocrystal Form A of Compound I

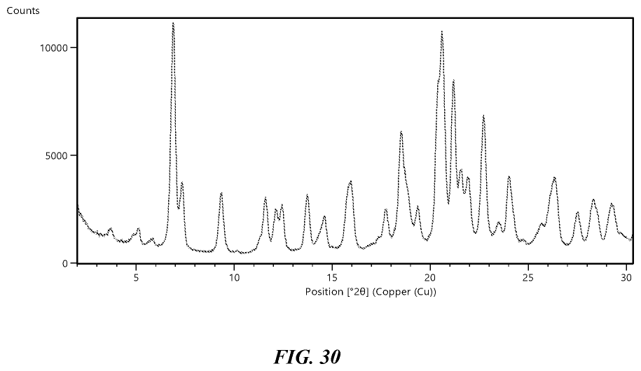

One embodiment of the invention provides aspartame cocrystal Form A of Compound I. In some embodiments, the aspartame cocrystal Form A of Compound I is substantially pure. In some embodiments, the aspartame cocrystal Form is characterized by an X-ray powder diffractogram substantially similar to that in FIG. 30. In some embodiments, the aspartame cocrystal Form A of Compound I is characterized by an X-ray powder diffractogram having a signal at one or more two-theta values selected from 22.7±0.2, 21.2±0.2, 20.6±0.2, 20.3±0.2, and 6.9±0.2. In some embodiments, the aspartame cocrystal Form A of Compound I is characterized by an X-ray powder diffractogram having a signal at the following two-theta values 22.7±0.2, 21.2±0.2, 20.6±0.2, 20.3±0.2, and 6.9±0.2. In some embodiments, the aspartame cocrystal Form A of Compound I is characterized by an X-ray powder diffractogram having (a) a signal at the following two-theta values 22.7±0.2, 21.2±0.2, 20.6±0.2, 20.3±0.2, and 6.9±0.2; and (b) a signal at one or more of the following two-theta values selected from 24.0±0.2, 21.6±0.2, 18.5±0.2, 16.0±0.2, and 7.4±0.2. In some embodiments, the aspartame cocrystal Form A of Compound I is characterized by an X-ray powder diffractogram having signals at the following two-theta values 24.0±0.2, 22.7±0.2, 21.6±0.2, 21.2±0.2, 20.6±0.2, 20.3±0.2, 18.5±0.2, 16.0±0.2, 7.4±0.2, and 6.9±0.2.

### Glutaric Acid Cocrystal Form A of Compound I

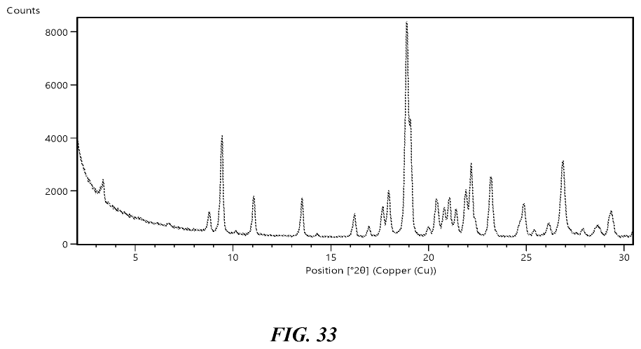

One embodiment of the invention provides glutaric acid cocrystal Form A of Compound I. In some embodiments, the glutaric acid cocrystal Form A of Compound I is substantially pure. In some embodiments, the glutaric acid cocrystal Form is characterized by an X-ray powder diffractogram substantially similar to that in FIG. 33. In some embodiments, the glutaric acid cocrystal Form A of Compound I is characterized by an X-ray powder diffractogram having a signal at one or more two-theta values selected from 26.9±0.2, 22.2±0.2, 19.1±0.2, 18.9±0.2, and 9.4±0.2. In some embodiments, the glutaric acid cocrystal Form A of Compound I is characterized by an X-ray powder diffractogram having a signal at the following two-theta values 26.9±0.2, 22.2±0.2, 19.1±0.2, 18.9±0.2, and 9.4±0.2. In some embodiments, the glutaric acid cocrystal Form A of Compound I is characterized by an X-ray powder diffractogram having (a) a signal at the following two-theta values 26.9±0.2, 22.2±0.2, 19.1±0.2, 18.9±0.2, and 9.4±0.2; and (b) a signal at one or more of the following two-theta values selected from 23.2±0.2, 21.9±0.2, 18.0±0.2, 13.5±0.2, and 11.0±0.2. In some embodiments, the glutaric acid cocrystal Form A of Compound I is characterized by an X-ray powder diffractogram having signals at the following two-theta values 26.9±0.2, 23.2±0.2, 22.2±0.2, 21.9±0.2, 19.1±0.2, 18.9±0.2, 18.0±0.2, 13.5±0.2, 11.0±0.2, and 9.4±0.22.

### L-Proline Cocrystal Form A of Compound I

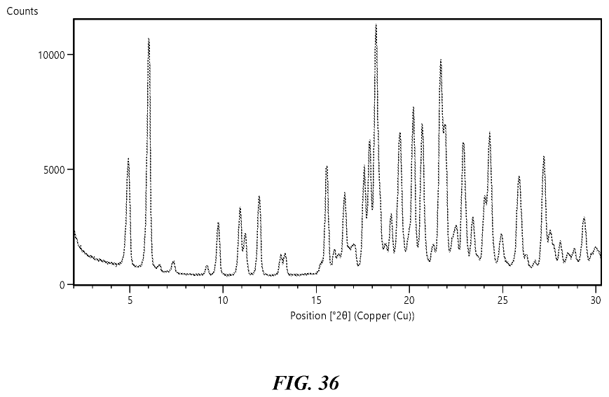

One embodiment of the invention provides L-proline cocrystal Form A of Compound I. In some embodiments, the L-proline cocrystal Form A of Compound I is substantially pure. In some embodiments, the L-proline cocrystal Form A is characterized by an X-ray powder diffractogram substantially similar to that in FIG. 36. In some embodiments, the L-proline cocrystal Form A of Compound I is characterized by an X-ray powder diffractogram having a signal at one or more two-theta values selected from 22.9±0.2, 20.2±0.2, 6.0±0.2, and 4.9±0.2. In some embodiments, the L-proline cocrystal Form A of Compound I is characterized by an X-ray powder diffractogram having a signal at the following two-theta values 22.9±0.2, 20.2±0.2, 6.0±0.2, and 4.9±0.2. In some embodiments, the L-proline cocrystal Form A of Compound I is characterized by an X-ray powder diffractogram having (a) a signal at the following two-theta values 22.9±0.2, 20.2±0.2, 6.0±0.2, and 4.9±0.2; and (b) a signal at one or more of the following two-theta values selected from 24.3±0.2, 22.0±0.2, 19.5±0.2, and 17.9±0.2. In some embodiments, the L-proline cocrystal Form A of Compound I is characterized by an X-ray powder diffractogram having signals at the following two-theta values 24.3±0.2, 22.9±0.2, 22.0±0.2, 20.2±0.2, 19.5±0.2, 17.9±0.2, 6.0±0.2, and 4.9±0.2.

### L-Proline Cocrystal Form B of Compound I

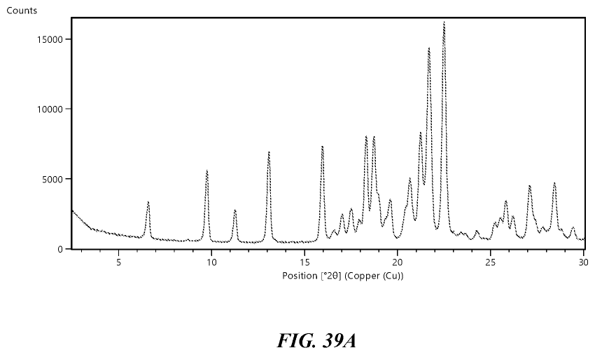

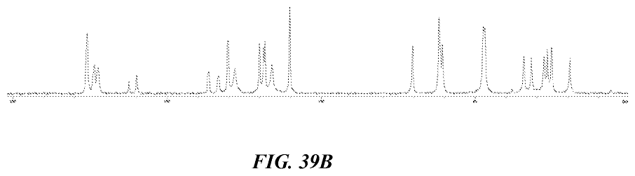

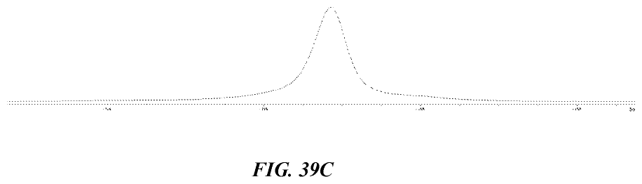

One embodiment of the invention provides L-proline cocrystal Form B of Compound I. In some embodiments, the L-proline cocrystal Form B of Compound I is substantially pure. In some embodiments, the L-proline cocrystal Form B is characterized by an X-ray powder diffractogram substantially similar to that in FIG. 39A. In some embodiments, the L-proline cocrystal Form B of Compound I is characterized by an X-ray powder diffractogram having a signal at one or more two-theta values selected from 22.5±0.2, 21.2±0.2, and 18.7±0.2. In some embodiments, the L-proline cocrystal Form B of Compound I is characterized by an X-ray powder diffractogram having a signal at the following two-theta values 22.5±0.2, 21.2±0.2, and 18.7±0.2. In some embodiments, the L-proline cocrystal Form B of Compound I is characterized by an X-ray powder diffractogram having (a) a signal at the following two-theta values 22.5±0.2, 21.2±0.2, and 18.7±0.2; and (b) a signal at one or more of the following two-theta values selected from 28.5±0.2, 16.0±0.2, and 13.1±0.2. In some embodiments, the L-proline cocrystal Form B of Compound I is characterized by an X-ray powder diffractogram having signals at the following two-theta values 28.5±0.2, 22.5±0.2, 21.2±0.2, 18.7±0.2, 16.0±0.2, and 13.1±0.2.

In some embodiments, the L-proline cocrystal Form B of Compound I is characterized by a ¹³C NMR spectrum having a signal at at least one, at least two, at least three, at least four, or at least five ppm value(s) chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm. In some embodiments, the L-proline cocrystal Form B of Compound I is characterized by a ¹³C NMR spectrum having a signal at at least seven, at least ten, at least twelve or at least fifteen ppm value(s) chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm. In some embodiments, the L-proline cocrystal Form B is characterized by a ¹³C NMR spectrum substantially similar to that in FIG. 39B.

In some embodiments, the L-proline cocrystal Form B of Compound I is characterized by a ¹⁹F NMR spectrum having a signal at −116.9±0.2 ppm. In some embodiments, the L-proline cocrystal Form B is characterized by a ¹⁹F NMR spectrum substantially similar to that in FIG. 39C.

### Vanillin Cocrystal Form a of Compound I

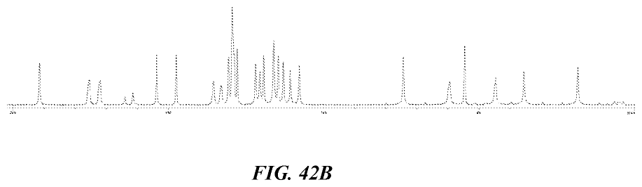

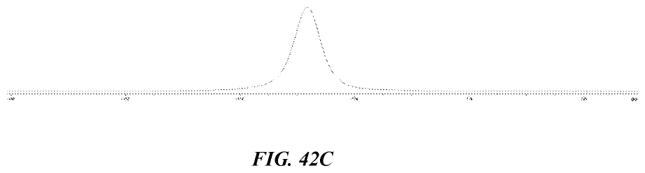

One embodiment of the invention provides vanillin cocrystal Form A of Compound I. In some embodiments, the vanillin cocrystal Form A of Compound I is substantially pure. In some embodiments, the vanillin cocrystal Form is characterized by an X-ray powder diffractogram substantially similar to that in FIG. 42A. In some embodiments, the vanillin cocrystal Form A of Compound I is characterized by an X-ray powder diffractogram having a signal at one or more two-theta values selected from 24.5 0.2, 21.9±0.2, 21.0±0.2, 15.6±0.2, and 9.6±0.2. In some embodiments, the vanillin cocrystal Form A of Compound I is characterized by an X-ray powder diffractogram having a signal at the following two-theta values 24.5±0.2, 21.9±0.2, 21.0±0.2, 15.6±0.2, and 9.6±0.2. In some embodiments, the vanillin cocrystal Form A of Compound I is characterized by an X-ray powder diffractogram having (a) a signal at the following two-theta values 24.5±0.2, 21.9±0.2, 21.0±0.2, 15.6±0.2, and 9.6±0.2; and (b) a signal at one or more of the following two-theta values selected from 27.4±0.2, 26.7±0.2, 26.2±0.2, 23.7±0.2, and 14.3±0.2. In some embodiments, the vanillin cocrystal Form A of Compound I is characterized by an X-ray powder diffractogram having signals at the following two-theta values 27.4±0.2, 26.7±0.2, 26.2±0.2, 24.5±0.2, 23.7±0.2, 21.9±0.2, 21.0±0.2, 15.6±0.2, 14.3±0.2, and 9.6±0.2.

In some embodiments, the vanillin cocrystal Form A of Compound I is characterized by a ¹³C NMR spectrum having a signal at at least one, at least two, at least three, at least four, or at least five ppm value(s) chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm. In some embodiments, the vanillin cocrystal Form A of Compound I is characterized by a ¹³C NMR spectrum having a signal at at least seven, at least ten, at least twelve or at least fifteen ppm value(s) chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm. In some embodiments, the vanillin cocrystal Form A is characterized by a ¹³C NMR spectrum substantially similar to that in FIG. 42B.

In some embodiments, the vanillin cocrystal Form A of Compound I is characterized by a ¹⁹F NMR spectrum having a signal at −115.2±0.2 ppm. In some embodiments, the vanillin cocrystal Form A is characterized by a ¹⁹F NMR spectrum substantially similar to that in FIG. 42C.

### 2-Pyridone Cocrystal Form A of Compound I

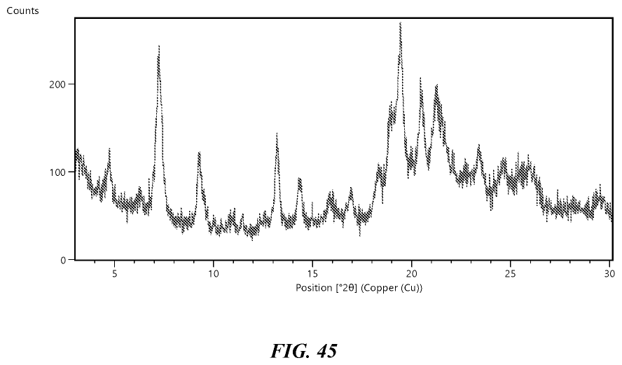

One embodiment of the invention provides 2-pyridone cocrystal Form A of Compound I. In some embodiments, the 2-pyridone cocrystal Form A of Compound I is substantially pure. In some embodiments, the 2-pyridone cocrystal Form is characterized by an X-ray powder diffractogram substantially similar to that in FIG. 45. In some embodiments, the 2-pyridone cocrystal Form A of Compound I is characterized by an X-ray powder diffractogram having a signal at one or more two-theta values selected from 19.5±0.2, 18.9±0.2, 15.8±0.2, 13.2±0.2, and 7.2±0.2. In some embodiments, the 2-pyridone cocrystal Form A of Compound I is characterized by an X-ray powder diffractogram having a signal at two or more of the following two-theta values 19.5±0.2, 18.9±0.2, 15.8±0.2, 13.2±0.2, and 7.2±0.2. In some embodiments, the 2-pyridone cocrystal Form A of Compound I is characterized by an X-ray powder diffractogram having a signal at three or more of the following two-theta values 19.5±0.2, 18.9±0.2, 15.8±0.2, 13.2±0.2, and 7.2±0.2 In some embodiments, the 2-pyridone cocrystal Form A of Compound I is characterized by an X-ray powder diffractogram having signals at the following two-theta values 19.5±0.2, 18.9±0.2, 15.8±0.2, 13.2±0.2, and 7.2±0.2.

In some embodiments, the 2-pyridone cocrystal Form A of Compound I is characterized by a ¹³C NMR spectrum having one or more signals selected from 165.3±0.2 ppm, 136.1±0.2 ppm, 129.7±0.2 ppm, and 119.8±0.2 ppm. In some embodiments, the 2-pyridone cocrystal Form A of Compound I is characterized by a ¹³C NMR spectrum having signals at 165.3±0.2 ppm, 136.1±0.2 ppm, 129.7±0.2 ppm, and 119.8±0.2 ppm. In some embodiments, the 2-pyridone cocrystal Form A of Compound I is characterized by a ¹³C NMR spectrum having (a) signals at 165.3±0.2 ppm, 136.1±0.2 ppm, 129.7±0.2 ppm, and 119.8±0.2 ppm; and (b) one or more signals selected from 142.3±0.2 ppm, 135.2±0.2 ppm, 107.8±0.2 ppm, and 36.6±0.2 ppm. In some embodiments, the 2-pyridone cocrystal Form A of Compound I is characterized by a ¹³C NMR spectrum having signals at 165.3±0.2 ppm, 142.3±0.2 ppm, 136.1±0.2 ppm, 135.2±0.2 ppm, 129.7±0.2 ppm, 119.8±0.2 ppm, 107.8±0.2 ppm, and 36.6±0.2 ppm.

In some embodiments, the 2-pyridone cocrystal Form A of Compound I is characterized by a ¹⁹F NMR spectrum having a signal at −112.1±0.2 ppm or −115.5±0.2 ppm. In some embodiments, the 2-pyridone cocrystal Form A of Compound I is characterized by a ¹⁹F NMR spectrum having signals at −112.1±0.2 ppm, and −115.5±0.2 ppm.

Another aspect of the disclosure provides pharmaceutical compositions comprising a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A. In some embodiments, the pharmaceutical composition comprising a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, can be administered as a separate composition concurrently with, prior to, or subsequent to, a composition comprising at least one other active therapeutic agent. In some embodiments, a pharmaceutical composition comprising a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A, can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science andPractice ofPharmacy, 21st edition, 2005, ed. D. B. Troy, Lippincott Williams & Wilkins, Philadelphia, and Encyclopedia ofPharmaceutical Technology, eds. J. Swarbrick and J C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

In some embodiments, Compound I is a crystalline solid consisting of 1% to 99% Form B relative to the total weight of the crystalline solid Compound I. In some embodiments, the crystalline solid consists of 2% to 99% Form B relative to the total weight of the crystalline solid Compound I. In some embodiments, the crystalline solid consists of 5% to 99% Form B relative to the total weight of the crystalline solid Compound I. In some embodiments, the crystalline solid consists of 10% to 99% Form B relative to the total weight of the crystalline solid Compound I. In some embodiments, the crystalline solid consists of 15% to 99% Form B relative to the total weight of the crystalline solid Compound I. In some embodiments, the crystalline solid consists of 20% to 99% Form B relative to the total weight of the crystalline solid Compound I. In some embodiments, the crystalline solid consists of 25% to 99% Form B relative to the total weight of the crystalline solid Compound I. In some embodiments, the crystalline solid consists of 30% to 99% Form B relative to the total weight of the crystalline solid Compound I. In some embodiments, the crystalline solid consists of 35% to 99% Form B relative to the total weight of the crystalline solid Compound I. In some embodiments, the crystalline solid consists of 45% to 99% Form B relative to the total weight of the crystalline solid Compound I. In some embodiments, the crystalline solid consists of 50% to 99% Form B relative to the total weight of the crystalline solid Compound I. In some embodiments, the crystalline solid consists of 55% to 99% Form B relative to the total weight of the crystalline solid Compound I. In some embodiments, the crystalline solid consists of 60% to 99% Form B relative to the total weight of the crystalline solid Compound I. In some embodiments, the crystalline solid consists of 65% to 99% Form B relative to the total weight of the crystalline solid Compound I. In some embodiments, the crystalline solid consists of 70% to 99% Form B relative to the total weight of the crystalline solid Compound I. In some embodiments, the crystalline solid consists of 75% to 99% Form B relative to the total weight of the crystalline solid Compound I. In some embodiments, the crystalline solid consists of 80% to 99% Form B relative to the total weight of the crystalline solid Compound I. In some embodiments, the crystalline solid consists of 85% to 99% Form B relative to the total weight of the crystalline solid Compound I. In some embodiments, the crystalline solid consists of 90% to 99% Form B relative to the total weight of the crystalline solid Compound I. In some embodiments, the crystalline solid consists of 95% to 99% Form B relative to the total weight of the crystalline solid Compound I.

In some embodiments, Compound I is a crystalline solid consisting of 1% to 99% citric acid cocrystal Form A relative to the total weight of the crystalline solid Compound I. In some embodiments, the crystalline solid consists of 2% to 99% citric acid cocrystal Form A relative to the total weight of the crystalline solid Compound I. In some embodiments, the crystalline solid consists of 5% to 99% citric acid cocrystal Form A relative to the total weight of the crystalline solid Compound I. In some embodiments, the crystalline solid consists of 10% to 99% citric acid cocrystal Form A relative to the total weight of the crystalline solid Compound I. In some embodiments, the crystalline solid consists of 15% to 99% citric acid cocrystal Form A relative to the total weight of the crystalline solid Compound I. In some embodiments, the crystalline solid consists of 20% to 99% citric acid cocrystal Form A relative to the total weight of the crystalline solid Compound I. In some embodiments, the crystalline solid consists of 25% to 99% citric acid cocrystal Form A relative to the total weight of the crystalline solid Compound I. In some embodiments, the crystalline solid consists of 30% to 99% citric acid cocrystal Form A relative to the total weight of the crystalline solid Compound I. In some embodiments, the crystalline solid consists of 35% to 99% citric acid cocrystal Form A relative to the total weight of the crystalline solid Compound I. In some embodiments, the crystalline solid consists of 45% to 99% citric acid cocrystal Form A relative to the total weight of the crystalline solid Compound I. In some embodiments, the crystalline solid consists of 50% to 99% citric acid cocrystal Form A relative to the total weight of the crystalline solid Compound I. In some embodiments, the crystalline solid consists of 55% to 99% citric acid cocrystal Form A relative to the total weight of the crystalline solid Compound I. In some embodiments, the crystalline solid consists of 60% to 99% citric acid cocrystal Form A relative to the total weight of the crystalline solid Compound I. In some embodiments, the crystalline solid consists of 65% to 99% citric acid cocrystal Form A relative to the total weight of the crystalline solid Compound I. In some embodiments, the crystalline solid consists of 70% to 99% citric acid cocrystal Form A relative to the total weight of the crystalline solid Compound I. In some embodiments, the crystalline solid consists of 75% to 99% citric acid cocrystal Form A relative to the total weight of the crystalline solid Compound I. In some embodiments, the crystalline solid consists of 80% to 99% citric acid cocrystal Form A relative to the total weight of the crystalline solid Compound I. In some embodiments, the crystalline solid consists of 85% to 99% citric acid cocrystal Form A relative to the total weight of the crystalline solid Compound I. In some embodiments, the crystalline solid consists of 90% to 99% citric acid cocrystal Form A relative to the total weight of the crystalline solid Compound I. In some embodiments, the crystalline solid consists of 95% to 99% citric acid cocrystal Form A relative to the total weight of the crystalline solid Compound I.

In some embodiments, Compound I is a crystalline solid consisting of 1% to 99% piperazine cocrystal Form A relative to the total weight of the crystalline solid Compound I. In some embodiments, the crystalline solid consists of 2% to 99% piperazine cocrystal Form A relative to the total weight of the crystalline solid Compound I. In some embodiments, the crystalline solid consists of 5% to 99% piperazine cocrystal Form A relative to the total weight of the crystalline solid Compound I. In some embodiments, the crystalline solid consists of 10% to 99% piperazine cocrystal Form A relative to the total weight of the crystalline solid Compound I. In some embodiments, the crystalline solid consists of 15% to 99% piperazine cocrystal Form A relative to the total weight of the crystalline solid Compound I. In some embodiments, the crystalline solid consists of 20% to 99% piperazine cocrystal Form A relative to the total weight of the crystalline solid Compound I. In some embodiments, the crystalline solid consists of 25% to 99% piperazine cocrystal Form A relative to the total weight of the crystalline solid Compound I. In some embodiments, the crystalline solid consists of 30% to 99% piperazine cocrystal Form A relative to the total weight of the crystalline solid Compound I. In some embodiments, the crystalline solid consists of 35% to 99% piperazine cocrystal Form A relative to the total weight of the crystalline solid Compound I. In some embodiments, the crystalline solid consists of 45% to 99% piperazine cocrystal Form A relative to the total weight of the crystalline solid Compound I. In some embodiments, the crystalline solid consists of 50% to 99% piperazine cocrystal Form A relative to the total weight of the crystalline solid Compound I. In some embodiments, the crystalline solid consists of 55% to 99% piperazine cocrystal Form A relative to the total weight of the crystalline solid Compound I. In some embodiments, the crystalline solid consists of 60% to 99% piperazine cocrystal Form A relative to the total weight of the crystalline solid Compound I. In some embodiments, the crystalline solid consists of 65% to 99% piperazine cocrystal Form A relative to the total weight of the crystalline solid Compound I. In some embodiments, the crystalline solid consists of 70% to 99% piperazine cocrystal Form A relative to the total weight of the crystalline solid Compound I. In some embodiments, the crystalline solid consists of 75% to 99% piperazine cocrystal Form A relative to the total weight of the crystalline solid Compound I. In some embodiments, the crystalline solid consists of 80% to 99% piperazine cocrystal Form A relative to the total weight of the crystalline solid Compound I. In some embodiments, the crystalline solid consists of 85% to 99% piperazine cocrystal Form A relative to the total weight of the crystalline solid Compound I. In some embodiments, the crystalline solid consists of 90% to 99% piperazine cocrystal Form A relative to the total weight of the crystalline solid Compound I. In some embodiments, the crystalline solid consists of 95% to 99% piperazine cocrystal Form A relative to the total weight of the crystalline solid Compound I.

In some embodiments, Compound I is a crystalline solid consisting of 1% to 99% urea cocrystal Form A relative to the total weight of the crystalline solid Compound I. In some embodiments, the crystalline solid consists of 2% to 99% urea cocrystal Form A relative to the total weight of the crystalline solid Compound I. In some embodiments, the crystalline solid consists of 5% to 99% urea cocrystal Form A relative to the total weight of the crystalline solid Compound I. In some embodiments, the crystalline solid consists of 10% to 99% urea cocrystal Form A relative to the total weight of the crystalline solid Compound I. In some embodiments, the crystalline solid consists of 15% to 99% urea cocrystal Form A relative to the total weight of the crystalline solid Compound I. In some embodiments, the crystalline solid consists of 20% to 99% urea cocrystal Form A relative to the total weight of the crystalline solid Compound I. In some embodiments, the crystalline solid consists of 25% to 99% urea cocrystal Form A relative to the total weight of the crystalline solid Compound I. In some embodiments, the crystalline solid consists of 30% to 99% urea cocrystal Form A relative to the total weight of the crystalline solid Compound I. In some embodiments, the crystalline solid consists of 35% to 99% urea cocrystal Form A relative to the total weight of the crystalline solid Compound I. In some embodiments, the crystalline solid consists of 45% to 99% urea cocrystal Form A relative to the total weight of the crystalline solid Compound I. In some embodiments, the crystalline solid consists of 50% to 99% urea cocrystal Form A relative to the total weight of the crystalline solid Compound I. In some embodiments, the crystalline solid consists of 55% to 99% urea cocrystal Form A relative to the total weight of the crystalline solid Compound I. In some embodiments, the crystalline solid consists of 60% to 99% urea cocrystal Form A relative to the total weight of the crystalline solid Compound I. In some embodiments, the crystalline solid consists of 65% to 99% urea cocrystal Form A relative to the total weight of the crystalline solid Compound I. In some embodiments, the crystalline solid consists of 70% to 99% urea cocrystal Form A relative to the total weight of the crystalline solid Compound I. In some embodiments, the crystalline solid consists of 75% to 99% urea cocrystal Form A relative to the total weight of the crystalline solid Compound I. In some embodiments, the crystalline solid consists of 80% to 99% urea cocrystal Form A relative to the total weight of the crystalline solid Compound I. In some embodiments, the crystalline solid consists of 85% to 99% urea cocrystal Form A relative to the total weight of the crystalline solid Compound I. In some embodiments, the crystalline solid consists of 90% to 99% urea cocrystal Form A relative to the total weight of the crystalline solid Compound I. In some embodiments, the crystalline solid consists of 95% to 99% urea cocrystal Form A relative to the total weight of the crystalline solid Compound I.

In some embodiments, Compound I is a crystalline solid consisting of 1% to 99% nicotinamide cocrystal Form A relative to the total weight of the crystalline solid Compound I. In some embodiments, the crystalline solid consists of 2% to 99% nicotinamide cocrystal Form A relative to the total weight of the crystalline solid Compound I. In some embodiments, the crystalline solid consists of 5% to 99% nicotinamide cocrystal Form A relative to the total weight of the crystalline solid Compound I. In some embodiments, the crystalline solid consists of 10% to 99% nicotinamide cocrystal Form A relative to the total weight of the crystalline solid Compound I. In some embodiments, the crystalline solid consists of 15% to 99% nicotinamide cocrystal Form A relative to the total weight of the crystalline solid Compound I. In some embodiments, the crystalline solid consists of 20% to 99% nicotinamide cocrystal Form A relative to the total weight of the crystalline solid Compound I. In some embodiments, the crystalline solid consists of 25% to 99% nicotinamide cocrystal Form A relative to the total weight of the crystalline solid Compound I. In some embodiments, the crystalline solid consists of 30% to 99% nicotinamide cocrystal Form A relative to the total weight of the crystalline solid Compound I. In some embodiments, the crystalline solid consists of 35% to 99% nicotinamide cocrystal Form A relative to the total weight of the crystalline solid Compound I. In some embodiments, the crystalline solid consists of 45% to 99% nicotinamide cocrystal Form A relative to the total weight of the crystalline solid Compound I. In some embodiments, the crystalline solid consists of 50% to 99% nicotinamide cocrystal Form A relative to the total weight of the crystalline solid Compound I. In some embodiments, the crystalline solid consists of 55% to 99% nicotinamide cocrystal Form A relative to the total weight of the crystalline solid Compound I. In some embodiments, the crystalline solid consists of 60% to 99% nicotinamide cocrystal Form A relative to the total weight of the crystalline solid Compound I. In some embodiments, the crystalline solid consists of 65% to 99% nicotinamide cocrystal Form A relative to the total weight of the crystalline solid Compound I. In some embodiments, the crystalline solid consists of 70% to 99% nicotinamide cocrystal Form A relative to the total weight of the crystalline solid Compound I. In some embodiments, the crystalline solid consists of 75% to 99% nicotinamide cocrystal Form A relative to the total weight of the crystalline solid Compound I. In some embodiments, the crystalline solid consists of 80% to 99% nicotinamide cocrystal Form A relative to the total weight of the crystalline solid Compound I. In some embodiments, the crystalline solid consists of 85% to 99% nicotinamide cocrystal Form A relative to the total weight of the crystalline solid Compound I. In some embodiments, the crystalline solid consists of 90% to 99% nicotinamide cocrystal Form A relative to the total weight of the crystalline solid Compound I. In some embodiments, the crystalline solid consists of 95% to 99% nicotinamide cocrystal Form A relative to the total weight of the crystalline solid Compound I.

In some embodiments, Compound I is a crystalline solid consisting of 1% to 99% nicotinamide cocrystal Form B relative to the total weight of the crystalline solid Compound I. In some embodiments, the crystalline solid consists of 2% to 99% nicotinamide cocrystal Form B relative to the total weight of the crystalline solid Compound I. In some embodiments, the crystalline solid consists of 5% to 99% nicotinamide cocrystal Form B relative to the total weight of the crystalline solid Compound I. In some embodiments, the crystalline solid consists of 10% to 99% nicotinamide cocrystal Form B relative to the total weight of the crystalline solid Compound I. In some embodiments, the crystalline solid consists of 15% to 99% nicotinamide cocrystal Form B relative to the total weight of the crystalline solid Compound I. In some embodiments, the crystalline solid consists of 20% to 99% nicotinamide cocrystal Form B relative to the total weight of the crystalline solid Compound I. In some embodiments, the crystalline solid consists of 25% to 99% nicotinamide cocrystal Form B relative to the total weight of the crystalline solid Compound I. In some embodiments, the crystalline solid consists of 30% to 99% nicotinamide cocrystal Form B relative to the total weight of the crystalline solid Compound I. In some embodiments, the crystalline solid consists of 35% to 99% nicotinamide cocrystal Form B relative to the total weight of the crystalline solid Compound I. In some embodiments, the crystalline solid consists of 45% to 99% nicotinamide cocrystal Form B relative to the total weight of the crystalline solid Compound I. In some embodiments, the crystalline solid consists of 50% to 99% nicotinamide cocrystal Form B relative to the total weight of the crystalline solid Compound I. In some embodiments, the crystalline solid consists of 55% to 99% nicotinamide cocrystal Form B relative to the total weight of the crystalline solid Compound I. In some embodiments, the crystalline solid consists of 60% to 99% nicotinamide cocrystal Form B relative to the total weight of the crystalline solid Compound I. In some embodiments, the crystalline solid consists of 65% to 99% nicotinamide cocrystal Form B relative to the total weight of the crystalline solid Compound I. In some embodiments, the crystalline solid consists of 70% to 99% nicotinamide cocrystal Form B relative to the total weight of the crystalline solid Compound I. In some embodiments, the crystalline solid consists of 75% to 99% nicotinamide cocrystal Form B relative to the total weight of the crystalline solid Compound I. In some embodiments, the crystalline solid consists of 80% to 99% nicotinamide cocrystal Form B relative to the total weight of the crystalline solid Compound I. In some embodiments, the crystalline solid consists of 85% to 99% nicotinamide cocrystal Form B relative to the total weight of the crystalline solid Compound I. In some embodiments, the crystalline solid consists of 90% to 99% nicotinamide cocrystal Form B relative to the total weight of the crystalline solid Compound I. In some embodiments, the crystalline solid consists of 95% to 99% nicotinamide cocrystal Form B relative to the total weight of the crystalline solid Compound I.

In some embodiments, Compound I is a crystalline solid consisting of 1% to 99% aspartame cocrystal Form A relative to the total weight of the crystalline solid Compound I. In some embodiments, the crystalline solid consists of 2% to 99% aspartame cocrystal Form A relative to the total weight of the crystalline solid Compound I. In some embodiments, the crystalline solid consists of 5% to 99% aspartame cocrystal Form A relative to the total weight of the crystalline solid Compound I. In some embodiments, the crystalline solid consists of 10% to 99% aspartame cocrystal Form A relative to the total weight of the crystalline solid Compound I. In some embodiments, the crystalline solid consists of 15% to 99% aspartame cocrystal Form A relative to the total weight of the crystalline solid Compound I. In some embodiments, the crystalline solid consists of 20% to 99% aspartame cocrystal Form A relative to the total weight of the crystalline solid Compound I. In some embodiments, the crystalline solid consists of 25% to 99% aspartame cocrystal Form A relative to the total weight of the crystalline solid Compound I. In some embodiments, the crystalline solid consists of 30% to 99% aspartame cocrystal Form A relative to the total weight of the crystalline solid Compound I. In some embodiments, the crystalline solid consists of 35% to 99% aspartame cocrystal Form A relative to the total weight of the crystalline solid Compound I. In some embodiments, the crystalline solid consists of 45% to 99% aspartame cocrystal Form A relative to the total weight of the crystalline solid Compound I. In some embodiments, the crystalline solid consists of 50% to 99% aspartame cocrystal Form A relative to the total weight of the crystalline solid Compound I. In some embodiments, the crystalline solid consists of 55% to 99% aspartame cocrystal Form A relative to the total weight of the crystalline solid Compound I. In some embodiments, the crystalline solid consists of 60% to 99% aspartame cocrystal Form A relative to the total weight of the crystalline solid Compound I. In some embodiments, the crystalline solid consists of 65% to 99% aspartame cocrystal Form A relative to the total weight of the crystalline solid Compound I. In some embodiments, the crystalline solid consists of 70% to 99% aspartame cocrystal Form A relative to the total weight of the crystalline solid Compound I. In some embodiments, the crystalline solid consists of 75% to 99% aspartame cocrystal Form A relative to the total weight of the crystalline solid Compound I. In some embodiments, the crystalline solid consists of 80% to 99% aspartame cocrystal Form A relative to the total weight of the crystalline solid Compound I. In some embodiments, the crystalline solid consists of 85% to 99% aspartame cocrystal Form A relative to the total weight of the crystalline solid Compound I. In some embodiments, the crystalline solid consists of 90% to 99% aspartame cocrystal Form A relative to the total weight of the crystalline solid Compound I. In some embodiments, the crystalline solid consists of 95% to 99% aspartame cocrystal Form A relative to the total weight of the crystalline solid Compound I.

In some embodiments, Compound I is a crystalline solid consisting of 1% to 99% glutaric acid cocrystal Form A relative to the total weight of the crystalline solid Compound I. In some embodiments, the crystalline solid consists of 2% to 99% glutaric acid cocrystal Form A relative to the total weight of the crystalline solid Compound I. In some embodiments, the crystalline solid consists of 5% to 99% glutaric acid cocrystal Form A relative to the total weight of the crystalline solid Compound I. In some embodiments, the crystalline solid consists of 10% to 99% glutaric acid cocrystal Form A relative to the total weight of the crystalline solid Compound I. In some embodiments, the crystalline solid consists of 15% to 99% glutaric acid cocrystal Form A relative to the total weight of the crystalline solid Compound I. In some embodiments, the crystalline solid consists of 20% to 99% glutaric acid cocrystal Form A relative to the total weight of the crystalline solid Compound I. In some embodiments, the crystalline solid consists of 25% to 99% glutaric acid cocrystal Form A relative to the total weight of the crystalline solid Compound I. In some embodiments, the crystalline solid consists of 30% to 99% glutaric acid cocrystal Form A relative to the total weight of the crystalline solid Compound I. In some embodiments, the crystalline solid consists of 35% to 99% glutaric acid cocrystal Form A relative to the total weight of the crystalline solid Compound I. In some embodiments, the crystalline solid consists of 45% to 99% glutaric acid cocrystal Form A relative to the total weight of the crystalline solid Compound I. In some embodiments, the crystalline solid consists of 50% to 99% glutaric acid cocrystal Form A relative to the total weight of the crystalline solid Compound I. In some embodiments, the crystalline solid consists of 55% to 99% glutaric acid cocrystal Form A relative to the total weight of the crystalline solid Compound I. In some embodiments, the crystalline solid consists of 60% to 99% glutaric acid cocrystal Form A relative to the total weight of the crystalline solid Compound I. In some embodiments, the crystalline solid consists of 65% to 99% glutaric acid cocrystal Form A relative to the total weight of the crystalline solid Compound I. In some embodiments, the crystalline solid consists of 70% to 99% glutaric acid cocrystal Form A relative to the total weight of the crystalline solid Compound I. In some embodiments, the crystalline solid consists of 75% to 99% glutaric acid cocrystal Form A relative to the total weight of the crystalline solid Compound I. In some embodiments, the crystalline solid consists of 80% to 99% glutaric acid cocrystal Form A relative to the total weight of the crystalline solid Compound I. In some embodiments, the crystalline solid consists of 85% to 99% glutaric acid cocrystal Form A relative to the total weight of the crystalline solid Compound I. In some embodiments, the crystalline solid consists of 90% to 99% glutaric acid cocrystal Form A relative to the total weight of the crystalline solid Compound I. In some embodiments, the crystalline solid consists of 95% to 99% glutaric acid cocrystal Form A relative to the total weight of the crystalline solid Compound I.

In some embodiments, Compound I is a crystalline solid consisting of 1% to 99% L-proline cocrystal Form A relative to the total weight of the crystalline solid Compound I. In some embodiments, the crystalline solid consists of 2% to 99% L-proline cocrystal Form A relative to the total weight of the crystalline solid Compound I. In some embodiments, the crystalline solid consists of 5% to 99% L-proline cocrystal Form A relative to the total weight of the crystalline solid Compound I. In some embodiments, the crystalline solid consists of 10% to 99% L-proline cocrystal Form A relative to the total weight of the crystalline solid Compound I. In some embodiments, the crystalline solid consists of 15% to 99% L-proline cocrystal Form A relative to the total weight of the crystalline solid Compound I. In some embodiments, the crystalline solid consists of 20% to 99% L-proline cocrystal Form A relative to the total weight of the crystalline solid Compound I. In some embodiments, the crystalline solid consists of 25% to 99% L-proline cocrystal Form A relative to the total weight of the crystalline solid Compound I. In some embodiments, the crystalline solid consists of 30% to 99% L-proline cocrystal Form A relative to the total weight of the crystalline solid Compound I. In some embodiments, the crystalline solid consists of 35% to 99% L-proline cocrystal Form A relative to the total weight of the crystalline solid Compound I. In some embodiments, the crystalline solid consists of 45% to 99% L-proline cocrystal Form A relative to the total weight of the crystalline solid Compound I. In some embodiments, the crystalline solid consists of 50% to 99% L-proline cocrystal Form A relative to the total weight of the crystalline solid Compound I. In some embodiments, the crystalline solid consists of 55% to 99% L-proline cocrystal Form A relative to the total weight of the crystalline solid Compound I. In some embodiments, the crystalline solid consists of 60% to 99% L-proline cocrystal Form A relative to the total weight of the crystalline solid Compound I. In some embodiments, the crystalline solid consists of 65% to 99% L-proline cocrystal Form A relative to the total weight of the crystalline solid Compound I. In some embodiments, the crystalline solid consists of 70% to 99% L-proline cocrystal Form A relative to the total weight of the crystalline solid Compound I. In some embodiments, the crystalline solid consists of 75% to 99% L-proline cocrystal Form A relative to the total weight of the crystalline solid Compound I. In some embodiments, the crystalline solid consists of 80% to 99% L-proline cocrystal Form A relative to the total weight of the crystalline solid Compound I. In some embodiments, the crystalline solid consists of 85% to 99% L-proline cocrystal Form A relative to the total weight of the crystalline solid Compound I. In some embodiments, the crystalline solid consists of 90% to 99% L-proline cocrystal Form A relative to the total weight of the crystalline solid Compound I. In some embodiments, the crystalline solid consists of 95% to 99% L-proline cocrystal Form A relative to the total weight of the crystalline solid Compound I.

In some embodiments, Compound I is a crystalline solid consisting of 1% to 99% L-proline cocrystal Form B relative to the total weight of the crystalline solid Compound I. In some embodiments, the crystalline solid consists of 2% to 99% L-proline cocrystal Form B relative to the total weight of the crystalline solid Compound I. In some embodiments, the crystalline solid consists of 5% to 99% L-proline cocrystal Form B relative to the total weight of the crystalline solid Compound I. In some embodiments, the crystalline solid consists of 10% to 99% L-proline cocrystal Form B relative to the total weight of the crystalline solid Compound I. In some embodiments, the crystalline solid consists of 15% to 99% L-proline cocrystal Form B relative to the total weight of the crystalline solid Compound I. In some embodiments, the crystalline solid consists of 20% to 99% L-proline cocrystal Form B relative to the total weight of the crystalline solid Compound I. In some embodiments, the crystalline solid consists of 25% to 99% L-proline cocrystal Form B relative to the total weight of the crystalline solid Compound I. In some embodiments, the crystalline solid consists of 30% to 99% L-proline cocrystal Form B relative to the total weight of the crystalline solid Compound I. In some embodiments, the crystalline solid consists of 35% to 99% L-proline cocrystal Form B relative to the total weight of the crystalline solid Compound I. In some embodiments, the crystalline solid consists of 45% to 99% L-proline cocrystal Form B relative to the total weight of the crystalline solid Compound I. In some embodiments, the crystalline solid consists of 50% to 99% L-proline cocrystal Form B relative to the total weight of the crystalline solid Compound I. In some embodiments, the crystalline solid consists of 55% to 99% L-proline cocrystal Form B relative to the total weight of the crystalline solid Compound I. In some embodiments, the crystalline solid consists of 60% to 99% L-proline cocrystal Form B relative to the total weight of the crystalline solid Compound I. In some embodiments, the crystalline solid consists of 65% to 99% L-proline cocrystal Form B relative to the total weight of the crystalline solid Compound I. In some embodiments, the crystalline solid consists of 70% to 99% L-proline cocrystal Form B relative to the total weight of the crystalline solid Compound I. In some embodiments, the crystalline solid consists of 75% to 99% L-proline cocrystal Form B relative to the total weight of the crystalline solid Compound I. In some embodiments, the crystalline solid consists of 80% to 99% L-proline cocrystal Form B relative to the total weight of the crystalline solid Compound I. In some embodiments, the crystalline solid consists of 85% to 99% L-proline cocrystal Form B relative to the total weight of the crystalline solid Compound I. In some embodiments, the crystalline solid consists of 90% to 99% L-proline cocrystal Form B relative to the total weight of the crystalline solid Compound I. In some embodiments, the crystalline solid consists of 95% to 99% L-proline cocrystal Form B relative to the total weight of the crystalline solid Compound I.

In some embodiments, Compound I is a crystalline solid consisting of 1% to 99% vanillin cocrystal Form A relative to the total weight of the crystalline solid Compound I. In some embodiments, the crystalline solid consists of 2% to 99% vanillin cocrystal Form A relative to the total weight of the crystalline solid Compound I. In some embodiments, the crystalline solid consists of 5% to 99% vanillin cocrystal Form A relative to the total weight of the crystalline solid Compound I. In some embodiments, the crystalline solid consists of 10% to 99% vanillin cocrystal Form A relative to the total weight of the crystalline solid Compound I. In some embodiments, the crystalline solid consists of 15% to 99% vanillin cocrystal Form A relative to the total weight of the crystalline solid Compound I. In some embodiments, the crystalline solid consists of 20% to 99% vanillin cocrystal Form A relative to the total weight of the crystalline solid Compound I. In some embodiments, the crystalline solid consists of 25% to 99% vanillin cocrystal Form A relative to the total weight of the crystalline solid Compound I. In some embodiments, the crystalline solid consists of 30% to 99% vanillin cocrystal Form A relative to the total weight of the crystalline solid Compound I. In some embodiments, the crystalline solid consists of 35% to 99% vanillin cocrystal Form A relative to the total weight of the crystalline solid Compound I. In some embodiments, the crystalline solid consists of 45% to 99% vanillin cocrystal Form A relative to the total weight of the crystalline solid Compound I. In some embodiments, the crystalline solid consists of 50% to 99% vanillin cocrystal Form A relative to the total weight of the crystalline solid Compound I. In some embodiments, the crystalline solid consists of 55% to 99% vanillin cocrystal Form A relative to the total weight of the crystalline solid Compound I. In some embodiments, the crystalline solid consists of 60% to 99% vanillin cocrystal Form A relative to the total weight of the crystalline solid Compound I. In some embodiments, the crystalline solid consists of 65% to 99% vanillin cocrystal Form A relative to the total weight of the crystalline solid Compound I. In some embodiments, the crystalline solid consists of 70% to 99% vanillin cocrystal Form A relative to the total weight of the crystalline solid Compound I. In some embodiments, the crystalline solid consists of 75% to 99% vanillin cocrystal Form A relative to the total weight of the crystalline solid Compound I. In some embodiments, the crystalline solid consists of 80% to 99% vanillin cocrystal Form A relative to the total weight of the crystalline solid Compound I. In some embodiments, the crystalline solid consists of 85% to 99% vanillin cocrystal Form A relative to the total weight of the crystalline solid Compound I. In some embodiments, the crystalline solid consists of 90% to 99% vanillin cocrystal Form A relative to the total weight of the crystalline solid Compound I. In some embodiments, the crystalline solid consists of 95% to 99% vanillin cocrystal Form A relative to the total weight of the crystalline solid Compound I.

In some embodiments, Compound I is a crystalline solid consisting of 1% to 99% 2-pyridone cocrystal Form A relative to the total weight of the crystalline solid Compound I. In some embodiments, the crystalline solid consists of 2% to 99% 2-pyridone cocrystal Form A relative to the total weight of the crystalline solid Compound I. In some embodiments, the crystalline solid consists of 5% to 99% 2-pyridone cocrystal Form A relative to the total weight of the crystalline solid Compound I. In some embodiments, the crystalline solid consists of 10% to 99% 2-pyridone cocrystal Form A relative to the total weight of the crystalline solid Compound I. In some embodiments, the crystalline solid consists of 15% to 99% 2-pyridone cocrystal Form A relative to the total weight of the crystalline solid Compound I. In some embodiments, the crystalline solid consists of 20% to 99% 2-pyridone cocrystal Form A relative to the total weight of the crystalline solid Compound I. In some embodiments, the crystalline solid consists of 25% to 99% 2-pyridone cocrystal Form A relative to the total weight of the crystalline solid Compound I. In some embodiments, the crystalline solid consists of 30% to 99% 2-pyridone cocrystal Form A relative to the total weight of the crystalline solid Compound I. In some embodiments, the crystalline solid consists of 35% to 99% 2-pyridone cocrystal Form A relative to the total weight of the crystalline solid Compound I. In some embodiments, the crystalline solid consists of 45% to 99% 2-pyridone cocrystal Form A relative to the total weight of the crystalline solid Compound I. In some embodiments, the crystalline solid consists of 50% to 99% 2-pyridone cocrystal Form A relative to the total weight of the crystalline solid Compound I. In some embodiments, the crystalline solid consists of 55% to 99% 2-pyridone cocrystal Form A relative to the total weight of the crystalline solid Compound I. In some embodiments, the crystalline solid consists of 60% to 99% 2-pyridone cocrystal Form A relative to the total weight of the crystalline solid Compound I. In some embodiments, the crystalline solid consists of 65% to 99% 2-pyridone cocrystal Form A relative to the total weight of the crystalline solid Compound I. In some embodiments, the crystalline solid consists of 70% to 99% 2-pyridone cocrystal Form A relative to the total weight of the crystalline solid Compound I. In some embodiments, the crystalline solid consists of 75% to 99% 2-pyridone cocrystal Form A relative to the total weight of the crystalline solid Compound I. In some embodiments, the crystalline solid consists of 80% to 99% 2-pyridone cocrystal Form A relative to the total weight of the crystalline solid Compound I. In some embodiments, the crystalline solid consists of 85% to 99% 2-pyridone cocrystal Form A relative to the total weight of the crystalline solid Compound I. In some embodiments, the crystalline solid consists of 90% to 99% 2-pyridone cocrystal Form A relative to the total weight of the crystalline solid Compound I. In some embodiments, the crystalline solid consists of 95% to 99% 2-pyridone cocrystal Form A relative to the total weight of the crystalline solid Compound I.

In some embodiments of the disclosure, a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is used to treat APOL1 mediated kidney disease. In some embodiments, the APOL1 mediated kidney disease is chosen from ESKD, FSGS, HIV-associated nephropathy, NDKD, arterionephrosclerosis, lupus nephritis, microalbuminuria, and chronic kidney disease. In some embodiments, the APOL1 mediated kidney disease treated with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is FSGS. In some embodiments, the APOL1 mediated kidney disease treated with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is NDKD. In some embodiments, the APOL1 mediated kidney disease treated with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A is ESKD. In some embodiments, the patient with APOL1 mediated kidney disease to be treated with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A has two APOL1 risk alleles. In some embodiments, the patient with APOL1 mediated kidney disease is homozygous forAPOL1 genetic risk alleles G1: S342G:I384M. In some embodiments, the patient with APOL1 mediated kidney disease is homozygous for APOL1 genetic risk alleles G2: N388del:Y389del. In some embodiments, the patient with APOL1 mediated kidney disease is heterozygous forAPOL1 genetic risk alleles G1: S342G:I384M and G2: N388del:Y389del.

In some embodiments, the methods of the disclosure comprise administering a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A to a patient in need thereof. In some embodiments, said patient in need thereof possesses APOL1 genetic variants, i.e., G1: S342G:I384M and G2: N388del:Y389del.

Another aspect of the disclosure provides methods of inhibiting APOL1 activity comprising contacting said APOL1 with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A. In some embodiments, the methods of inhibiting APOL1 activity comprise contacting said APOL1 with a solid form of Compound I selected from Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A.

Non-limiting Exemplary Embodiments

### 1. Form B of Compound I:

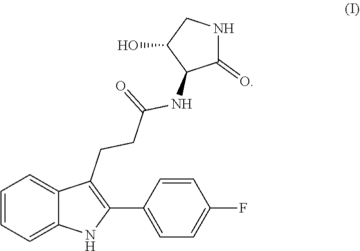 [SMILES: O=C(CCc1c(-c2ccc(F)cc2)[nH]c2ccccc12)N[C@@H]1C(=O)NC[C@H]1O]

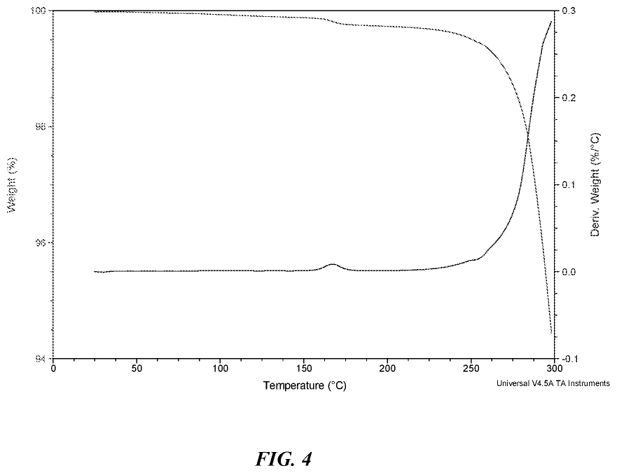

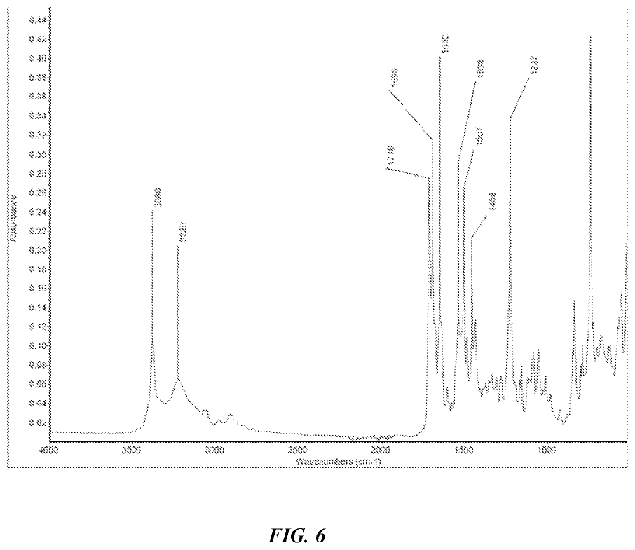

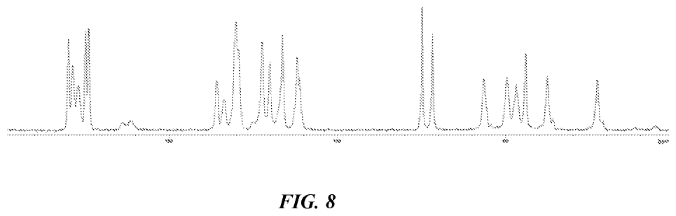

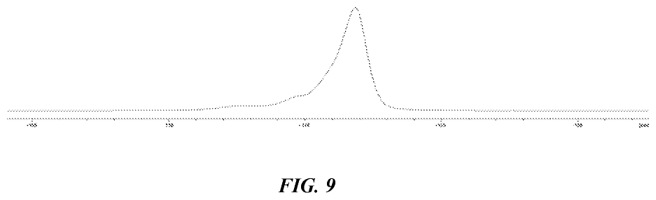

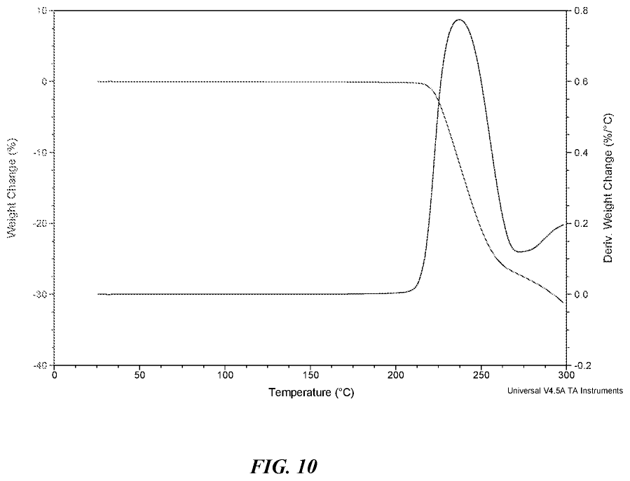

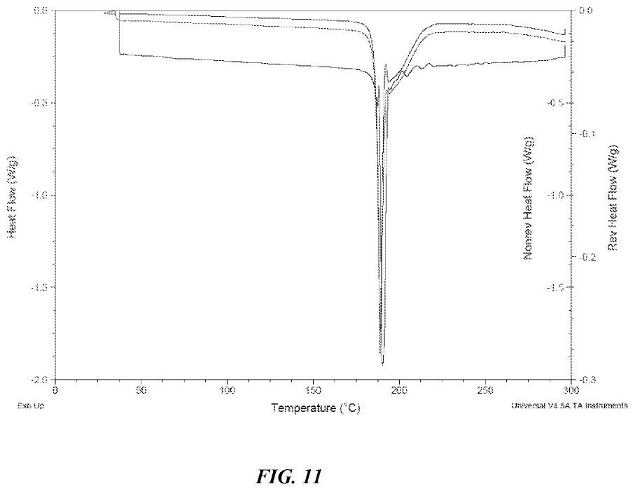

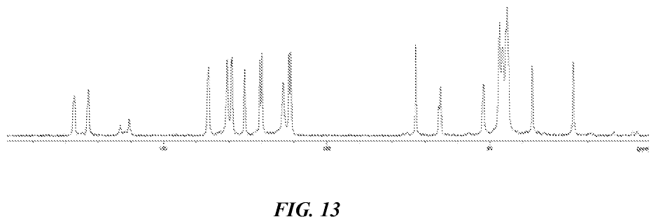

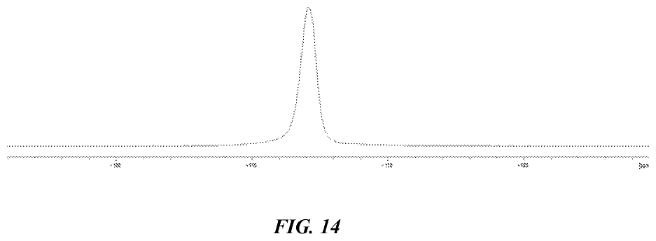

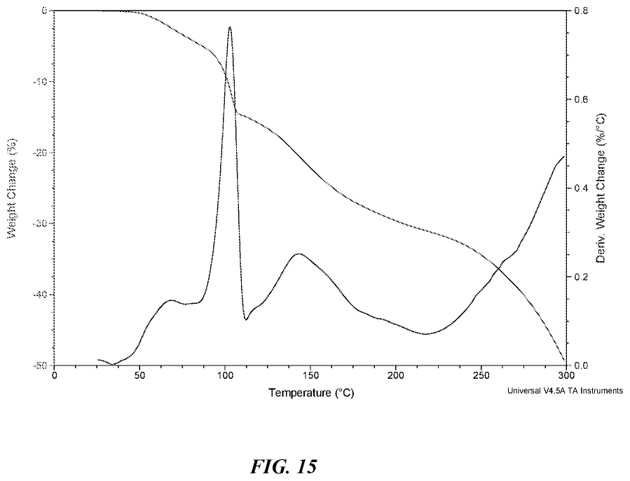

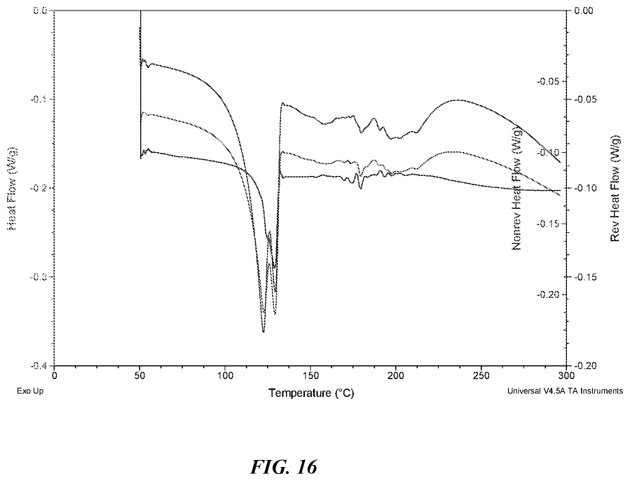

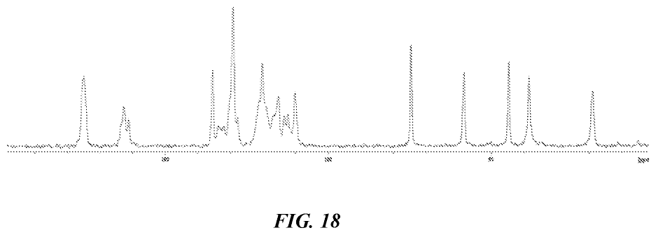

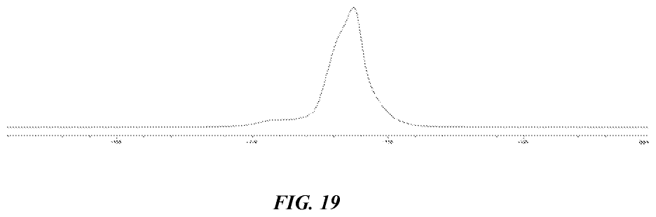

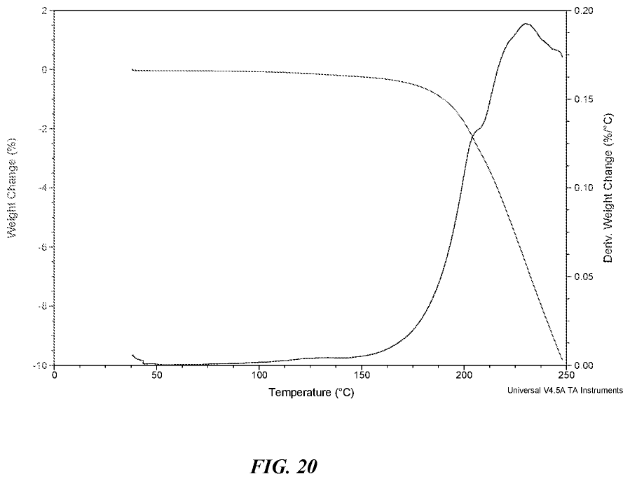

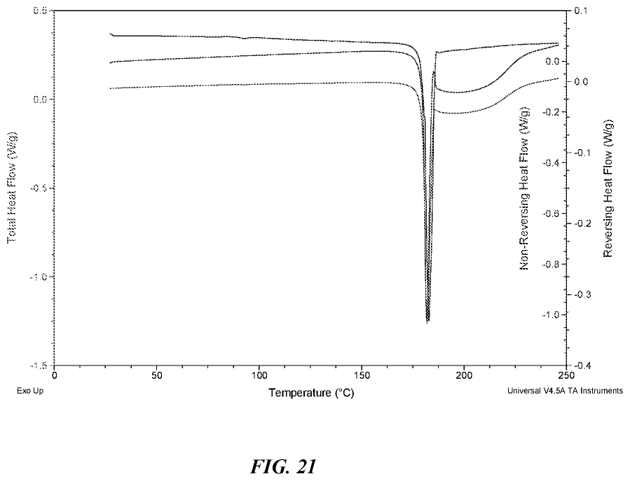

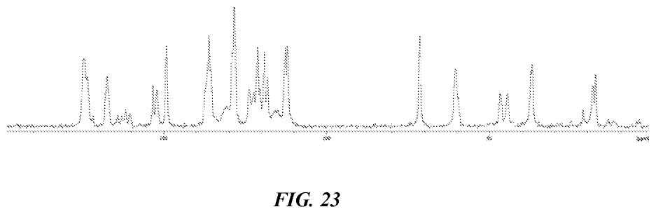

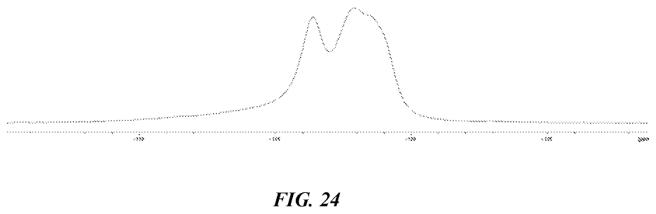

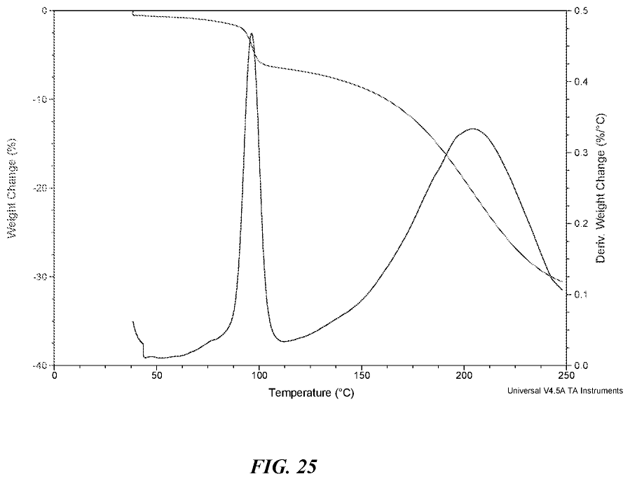

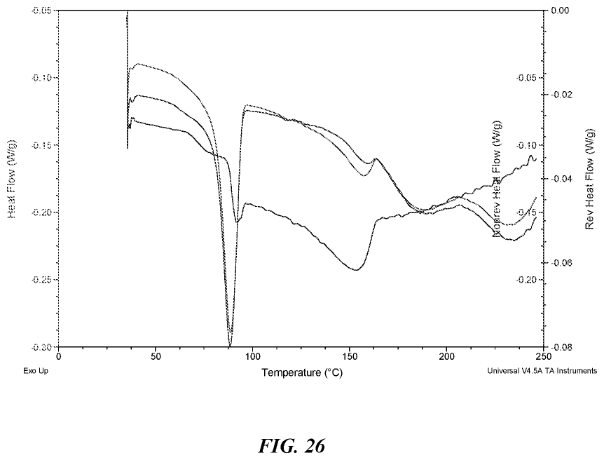

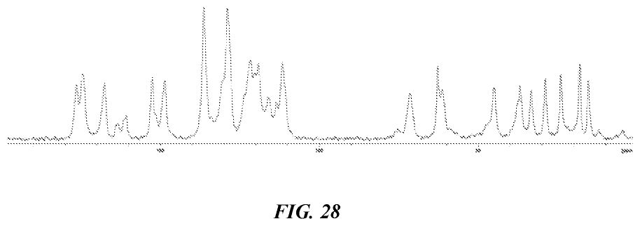

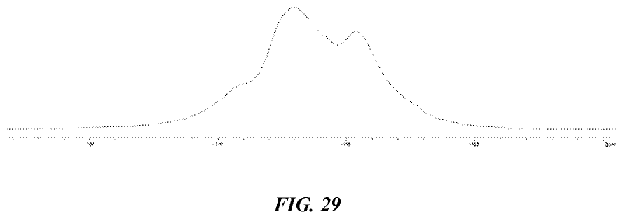

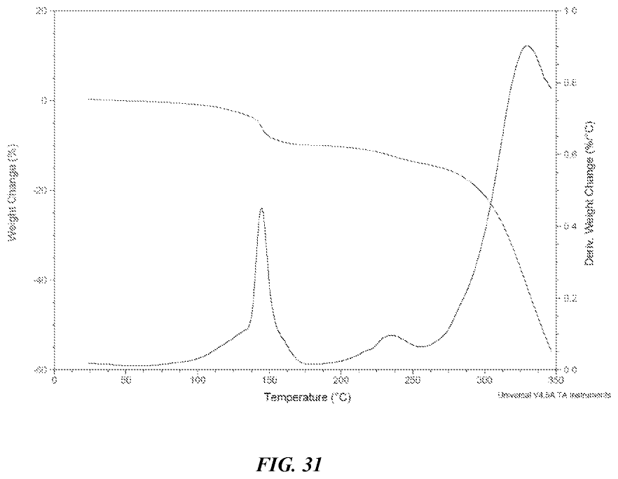

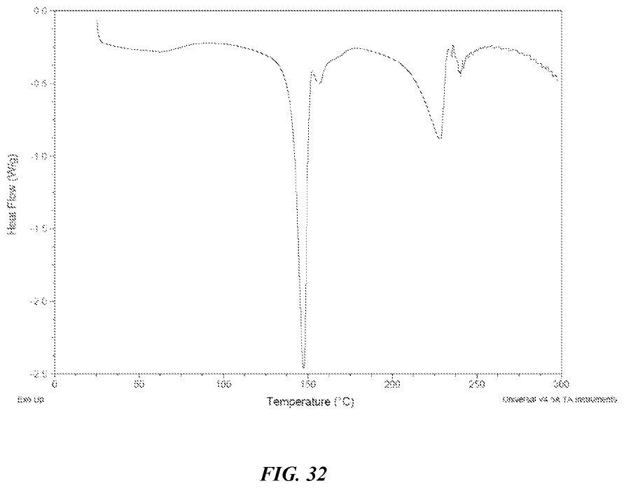

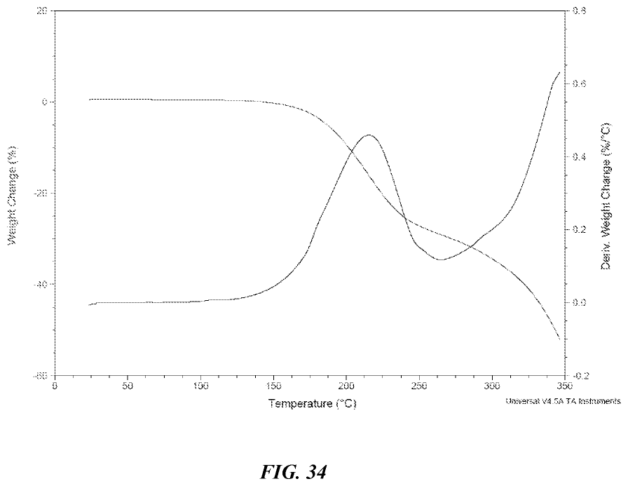

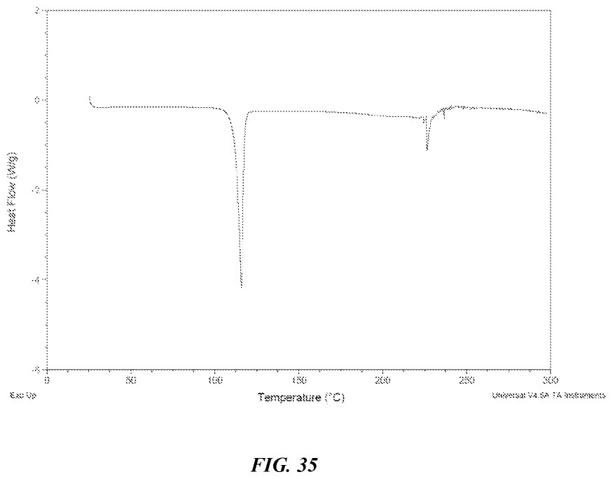

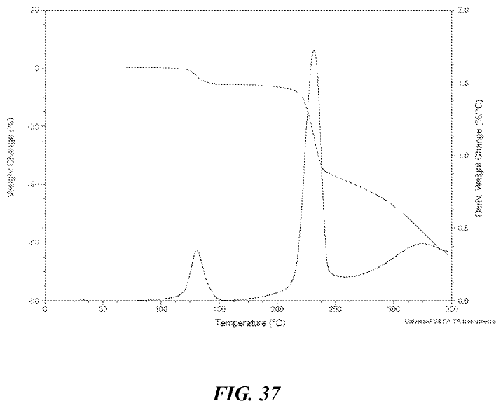

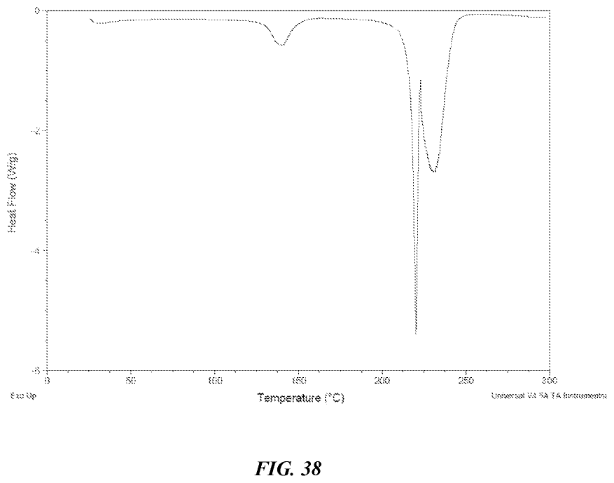

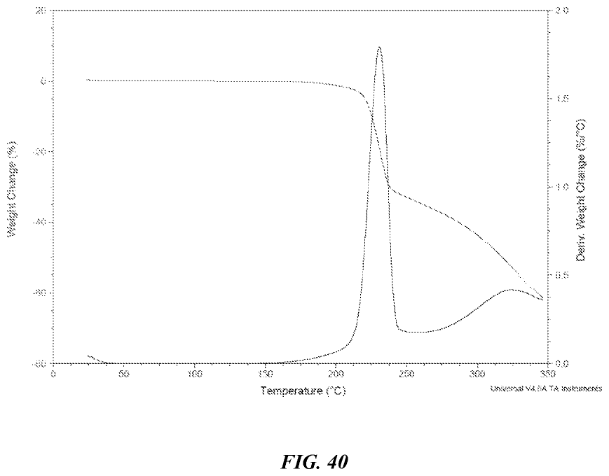

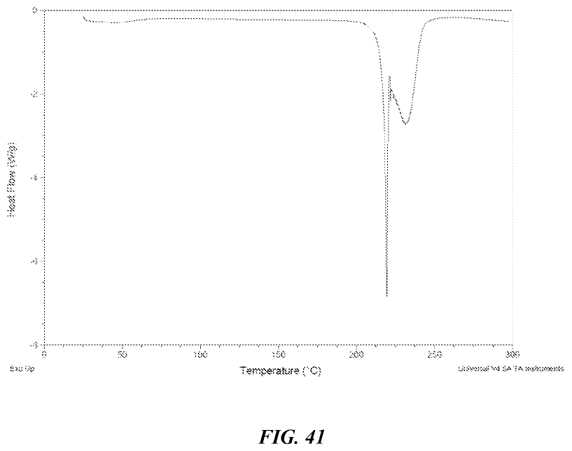

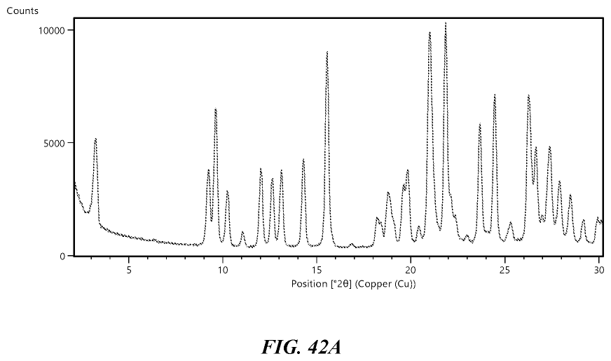

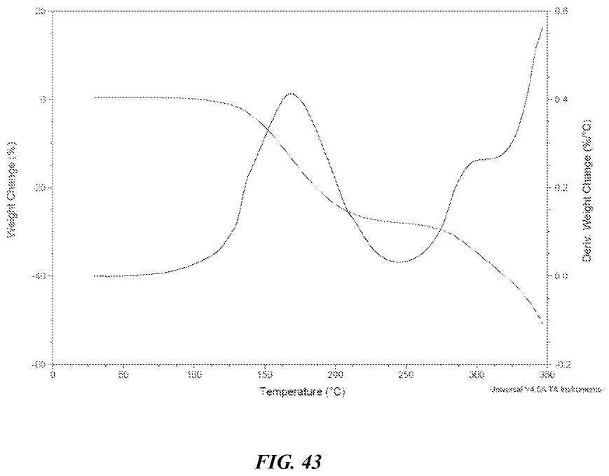

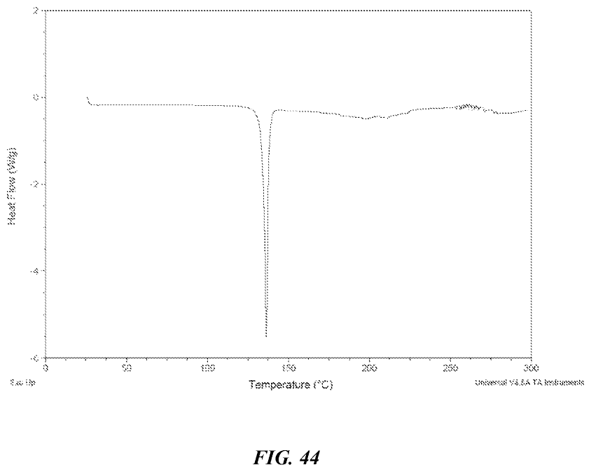

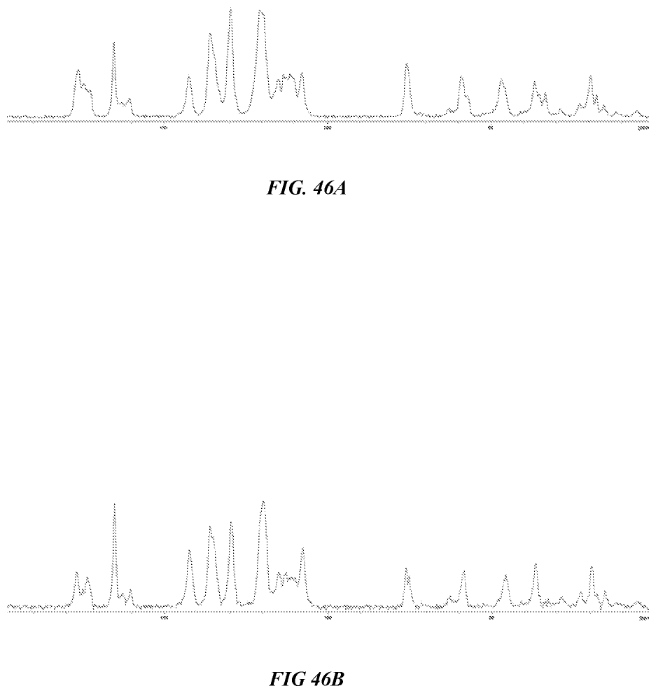

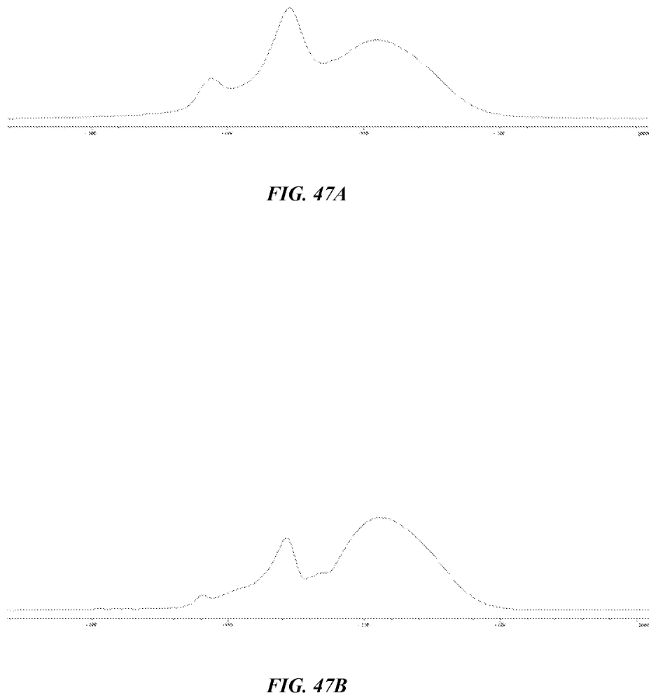

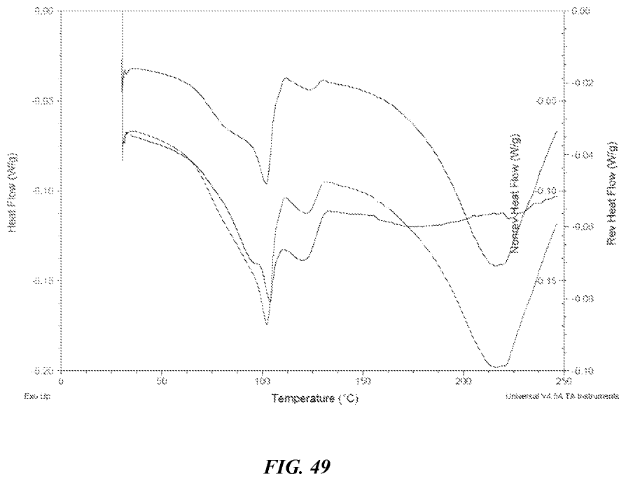

2. Form B of Compound I according to embodiment 1, characterized by an X-ray powder diffractogram substantially similar to that in FIG. 1A.
2a. Form B of Compound I according to embodiment 1, characterized by an X-ray powder diffractogram substantially similar to that in FIG. 1B.
2b. Form B of Compound I according to embodiment 1, characterized by an X-ray powder diffractogram substantially similar to that in FIG. 1C or FIG. 1D.
2c. Form B of Compound I according to embodiment 1, characterized by an X-ray powder diffractogram having a signal at 20.3±0.2, and a signal at least two two-theta values chosen from 4.7±0.2, 9.2±0.2, 14.2±0.2, 21.1±0.2, and 23.3±0.2.
3. Form B of Compound I according to embodiment 1, characterized by an X-ray powder diffractogram having a signal at at least two two-theta values chosen from 4.7±0.2, 9.2±0.2, 14.2±0.2, 20.3±0.2, 21.1±0.2, and 23.3±0.2.
4. Form B of Compound I according to embodiment 1, characterized by an X-ray powder diffractogram having a signal at 4.7±0.2, 9.2±0.2, 14.2±0.2, 20.3±0.2, 21.1±0.2, and 23.3±0.2 two-theta.
5. Form B of Compound I according to embodiment 1, characterized by an X-ray powder diffractogram having a signal at at least three two-theta values chosen from 4.7±0.2, 9.2±0.2, 14.2±0.2, 20.3±0.2, 21.1±0.2, and 23.3±0.2.
6. Form B of Compound I according to embodiment 1, characterized by an X-ray powder diffractogram having a signal at at least five two-theta values chosen from 4.7±0.2, 9.2±0.2, 14.2±0.2, 20.3±0.2, 21.1±0.2, and 23.3±0.2.
7. Form B of Compound I is characterized by an X-ray powder diffractogram having a signal at 4.7±0.2, 9.2±0.2, 14.2±0.2, 20.3±0.2, 21.1±0.2, and 23.3±0.2 two-theta.
7a. Form B of Compound I is characterized by an X-ray powder diffractogram having a signal at one or more two-theta values chosen from 16.9±0.2, 20.4±0.2, and 23.4±0.2.
7b. Form B of Compound I is characterized by an X-ray powder diffractogram having a signal at two or more, two-theta values chosen from 16.9±0.2, 20.4±0.2, and 23.4±0.2.
7c. Form B of Compound I is characterized by an X-ray powder diffractogram having a signal at 16.9±0.2, 20.4±0.2, and 23.4±0.2 two-theta.
7d. Form B of Compound I is characterized by an X-ray powder diffractogram (a) having a signal at one or more, two-theta values chosen from 16.9±0.2, 20.4±0.2, and 23.4±0.2; and (b) having a signal at one or more two-theta values chosen from 4.7±0.2, 9.3±0.2, 9.6±0.2, 14.3±0.2, and 21.2±0.2.
7e. Form B of Compound I is characterized by an X-ray powder diffractogram (a) having a signal at one or more, two-theta values chosen from 16.9±0.2, 20.4±0.2, and 23.4±0.2; and (b) having a signal at two or more two-theta values chosen from 4.7±0.2, 9.3±0.2, 9.6±0.2, 14.3±0.2, and 21.2±0.2.
7f. Form B of Compound I is characterized by an X-ray powder diffractogram (a) having a signal at one or more, two-theta values chosen from 16.9±0.2, 20.4±0.2, and 23.4±0.2; and (b) having a signal at three or more two-theta values chosen from 4.7±0.2, 9.3±0.2, 9.6±0.2, 14.3±0.2, and 21.2±0.2.
7g. Form B of Compound I is characterized by an X-ray powder diffractogram (a) having a signal at two or more, two-theta values chosen from 16.9±0.2, 20.4±0.2, and 23.4±0.2; and (b) having a signal at one or more two-theta values chosen from 4.7±0.2, 9.3±0.2, 9.6±0.2, 14.3±0.2, and 21.2±0.2.
7h. Form B of Compound I is characterized by an X-ray powder diffractogram (a) having a signal at two or more, two-theta values chosen from 16.9±0.2, 20.4±0.2, and 23.4±0.2; and (b) having a signal at two or more two-theta values chosen from 4.7±0.2, 9.3±0.2, 9.6±0.2, 14.3±0.2, and 21.2±0.2.
7i. Form B of Compound I is characterized by an X-ray powder diffractogram (a) having a signal at two or more, two-theta values chosen from 16.9±0.2, 20.4±0.2, and 23.4±0.2; and (b) having a signal at 4.7±0.2, 9.3±0.2, 9.6±0.2, 14.3±0.2, and 21.2±0.2 two-theta.
7j. Form B of Compound I is characterized by an X-ray powder diffractogram having a signal at 4.7±0.2, 9.3±0.2, 9.6±0.2, 14.3±0.2, 16.9±0.2, 20.4±0.2, 21.2±0.2and 23.4±0.2 two-theta.
8. Form B of Compound I, characterized by a ¹³C NMR spectrum having a signal at at least three ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.
9. Form B of Compound I according to any one of embodiments 1-8, characterized by a ¹³C NMR spectrum having a signal at at least five ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.
10. Form B of Compound I according to any one of embodiments 1-8, characterized by a ¹³C NMR spectrum having a signal at at least seven ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.
11. Form B of Compound I according to any one of embodiments 1-8, characterized by a ¹³C NMR spectrum having a signal at at least nine ppm values chosen from 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.
12. Form B of Compound I according to any one of embodiments 1-8, characterized by a ¹³C NMR spectrum having a signal at 132.9±0.2 ppm, 127.9±0.2 ppm, 114.7±0.2 ppm, 113.5±0.2 ppm, 59.2±0.2 ppm, 57.2±0.2 ppm, 33.3±0.2 ppm, 19.5±0.2 ppm, 17.6±0.2 ppm, and 16.7±0.2 ppm.
12a. Form B of Compound I characterized by a ¹³C NMR spectrum having a signal at two or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12b. Form B of Compound I characterized by a ¹³C NMR spectrum having a signal at three or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12c. Form B of Compound I characterized by a ¹³C NMR spectrum having a signal at five or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12d. Form B of Compound I characterized by a ¹³C NMR spectrum having a signal at seven or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12e. Form B of Compound I characterized by a ¹³C NMR spectrum having a signal at ten or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12f. Form B of Compound I characterized by a ¹³C NMR spectrum having a signal at twelve or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12g. Form B of Compound I characterized by a ¹³C NMR spectrum having a signal at fifteen or more ppm values chosen from 175.9±0.2 ppm, 172.3±0.2 ppm, 163.3±0.2 ppm, 161.9±0.2 ppm, 135.7±0.2 ppm, 134.2±0.2 ppm, 132.9±0.2 ppm, 130.1±0.2 ppm, 127.9±0.2 ppm, 124.3±0.2 ppm, 119.4±0.2 ppm, 118.2±0.2 ppm, 116.2±0.2 ppm, 114.7±0.2 ppm, 113.5±0.2 ppm, 111.5±0.2 ppm, 35.0±0.2 ppm, 33.3±0.2 ppm, 20.4±0.2 ppm, 19.5±0.2 ppm, and 17.6±0.2 ppm.
12h. Form B of Compound I according to embodiment 1, characterized by a ¹³C NMR spectrum substantially similar to that in FIG. 2.
13. Form B of Compound I according to embodiment 1, characterized by a ¹⁹F NMR spectrum having a signal at −112.5±0.2 ppm.
14. Form B of Compound I according to embodiment 1, characterized by a ¹⁹F NMR spectrum having signals at at least two ppm values chosen from −109.4±0.2 ppm, −112.5±0.2 ppm, and −113.7±0.2 ppm.
15. Form B of Compound I according to embodiment 1, characterized by a ¹⁹F NMR spectrum having signals at −109.4±0.2 ppm, −112.5±0.2 ppm, and −113.7±0.2 ppm.
16. Form B of Compound I according to embodiment 1, characterized by a DSC substantially similar to that in FIG. 5.
17. Form B of Compound I according to embodiment 1, characterized by a DSC having a melting onset of 168° C. and/or a peak at a temperature ranging from 167° C. to 171° C.
18. Form B of Compound I according to embodiment 1, characterized a TGA substantially similar to that in FIG. 4.
19. Form B of Compound I according to embodiment 1, characterized a TGA showing a weight loss of 0.3% w/w from ambient temperature up to 225° C.
20. Form B of Compound I according to embodiment 1, characterized by an IR spectrum substantially similar to that in FIG. 6.
21. A pharmaceutical composition comprising Form B of Compound I according to any one of embodiments 1 to 20 and a pharmaceutically acceptable carrier.
22. A method of treating APOL1 mediated kidney disease comprising administering to a patient in need thereof. Form B of Compound I according to any one of embodiments 1 to 20 or a pharmaceutical composition according to embodiment 21.
23. The method according to embodiment 22, wherein the APOL1 mediated kidney disease is chosen from ESKD, NDKD, FSGS, HIV-associated nephropathy, arterionephrosclerosis, lupus nephritis, microalbuminuria, and chronic kidney disease.
24. The method according to embodiment 22, wherein the APOL1 mediated kidney disease is chosen from ESKD, NDKD, and FSGS.
25. The method according to any one of embodiments 22-24, wherein the APOL1 mediated kidney disease is associated with APOL1 genetic alleles chosen from homozygous G1: S342G:I384M and homozygous G2: N388del:Y389del.
26. The method according to any one of embodiments 22-24, wherein the APOL1 mediated kidney disease is associated with compound heterozygous G1: S342G:I384M and G2: N388del:Y389del APOL1 genetic alleles.
27. A method of inhibiting APOL1 activity comprising contacting said APOL1 with at least one entity according to any one of claims 1 to 20 or a pharmaceutical composition according to embodiment 21.
28. The method according to embodiment 27, wherein the APOL1 is associated with APOL1 genetic alleles chosen from homozygous G: S342G:I384M and homozygous G2: N388del:Y389del.
29. The method according to embodiment 27, wherein the APOL1 is associated with compound heterozygous G1: S342G:I384M and G2: N388del:Y389del APOL1 genetic alleles.
30. A method of preparing Form B of Compound I comprising
mixing Compound I with n-pentanol at 65° C., and
stirring said mixture at 65° C. for at least 2 hours.
   31. The method according to embodiment 30, further comprising adding seeds of Form B of Compound I and/or n-heptane to said stirred mixture at 65° C.
   32. Citric acid cocrystal Form A cocrystal of Compound I.
   33. Citric acid cocrystal Form A of Compound I according to embodiment 32, characterized by an X-ray powder diffractogram having a signal at one or more two-theta values selected from 24.4±0.2 19.5±0.2, 14.6±0.2, and 4.9±0.2.
   34. Citric acid cocrystal Form A of Compound I according to embodiment 32, characterized by an X-ray powder diffractogram having a signal at two or more two-theta values selected from 24.4±0.2 19.5±0.2, 14.6±0.2, and 4.9±0.2.
   35. Citric acid cocrystal Form A of Compound I according to embodiment 32, characterized by an X-ray powder diffractogram having a signal at three or more two-theta values selected from 24.4±0.2 19.5±0.2, 14.6±0.2, and 4.9±0.2.
   36. Citric acid cocrystal Form A of Compound I according to embodiment 32, characterized by an X-ray powder diffractogram having signals at 24.4±0.2, 19.5±0.2, 14.6±0.2, and 4.9±0.2 two-theta.
   37. Citric acid cocrystal Form A of Compound I according to embodiment 32, characterized by an X-ray powder diffractogram having (a) a signal at the following two-theta values 24.4±0.2 19.5±0.2, 14.6±0.2, and 4.9±0.2; and (b) a signal at one or more two-theta values selected from 22.2±0.2, 21.2±0.2, 18.3±0.2, 18.2±0.2, and 9.2±0.2.
   38. Citric acid cocrystal Form A of Compound I according to embodiment 32, characterized by an X-ray powder diffractogram having (a) a signal at the following two-theta values 24.4±0.2 19.5±0.2, 14.6±0.2, and 4.9±0.2; and (b) a signal at two or more two-theta values selected from 22.2±0.2, 21.2±0.2, 18.3±0.2, 18.2±0.2, and 9.2±0.2.
   39. Citric acid cocrystal Form A of Compound I according to embodiment 32, characterized by an X-ray powder diffractogram having (a) a signal at the following two-theta values 24.4±0.2 19.5±0.2, 14.6±0.2, and 4.9±0.2; and (b) a signal at three or more two-theta values selected from 22.2±0.2, 21.2±0.2, 18.3±0.2, 18.2±0.2, and 9.2±0.2
   40. Citric acid cocrystal Form A of Compound I according to embodiment 32, characterized by an X-ray powder diffractogram having a signals at 24.4±0.2, 22.2±0.2, 21.2±0.2, 19.5±0.2, 18.3±0.2, 18.2±0.2, 14.6±0.2, 9.2±0.2, and 4.9±0.2 two-theta.
   41. Citric acid cocrystal Form A of Compound I according to embodiment 32, characterized by an X-ray powder diffractogram substantially similar to that in FIG. 7.
   42. Citric acid cocrystal Form A of Compound I according to embodiment 32, characterized by a ¹³C NMR spectrum having one or more signals selected from 174.8±0.2 ppm, 173.8±0.2 ppm, 130.1±0.2 ppm, 74.8±0.2 ppm, and 71.8±0.2 ppm.
   43. Citric acid cocrystal Form A of Compound I according to embodiment 32, characterized by a ¹³C NMR spectrum having two or more signals selected from 174.8±0.2 ppm, 173.8±0.2 ppm, 130.1±0.2 ppm, 74.8±0.2 ppm, and 71.8±0.2 ppm.
   44. Citric acid cocrystal Form A of Compound I according to embodiment 32, characterized by a ¹³C NMR spectrum having three or more signals selected from 174.8±0.2 ppm, 173.8±0.2 ppm, 130.1±0.2 ppm, 74.8±0.2 ppm, and 71.8±0.2 ppm.
   45. Citric acid cocrystal Form A of Compound I according to embodiment 32, characterized by a ¹³C NMR spectrum signals at 174.8±0.2 ppm, 173.8±0.2 ppm, 130.1±0.2 ppm, 74.8±0.2 ppm, and 71.8±0.2 ppm.
   46. Citric acid cocrystal Form A of Compound I according to embodiment 32, characterized by a ¹³C NMR spectrum having (a) signals at 174.8±0.2 ppm, 173.8±0.2 ppm, 130.1±0.2 ppm, 74.8±0.2 ppm, and 71.8±0.2 ppm; and (b) one or more signals selected from 179.9±0.2 ppm, 129.4±0.2 ppm, 122.4±0.2 ppm, 116.3±0.2 ppm, and 44.1±0.2 ppm.
   47. Citric acid cocrystal Form A of Compound I according to embodiment 32, characterized by a ¹³C NMR spectrum having (a) signals at 174.8±0.2 ppm, 173.8±0.2 ppm, 130.1±0.2 ppm, 74.8±0.2 ppm, and 71.8±0.2 ppm; and (b) two or more signals selected from 179.9±0.2 ppm, 129.4±0.2 ppm, 122.4±0.2 ppm, 116.3±0.2 ppm, and 44.1±0.2 ppm.
   48. Citric acid cocrystal Form A of Compound I according to embodiment 32, characterized by a ¹³C NMR spectrum having (a) signals at 174.8±0.2 ppm, 173.8±0.2 ppm, 130.1±0.2 ppm, 74.8±0.2 ppm, and 71.8±0.2 ppm; and (b) three or more signals selected from 179.9±0.2 ppm, 129.4±0.2 ppm, 122.4±0.2 ppm, 116.3±0.2 ppm, and 44.1±0.2 ppm.
   49. Citric acid cocrystal Form A of Compound I according to embodiment 32, characterized by a ¹³C NMR spectrum having signals at 179.9±0.2 ppm, 174.8±0.2 ppm, 173.8±0.2 ppm, 130.1±0.2 ppm, 129.4±0.2 ppm, 122.4±0.2 ppm, 116.3±0.2 ppm, 74.8±0.2 ppm, 71.8±0.2 ppm, and 44.1±0.2 ppm.
   50. Citric acid cocrystal Form A of Compound I according to embodiment 32, characterized by a ¹³C NMR spectrum substantially similar to that in FIG. 8.
   51. Citric acid cocrystal Form A of Compound I according to embodiment 32, characterized by a ¹⁹F NMR spectrum having a signal at one or more ppm values chosen from −112.6±0.2 ppm, −114.8±0.2 ppm, and −116.8±0.2 ppm.
   52. Citric acid cocrystal Form A of Compound I according to embodiment 32, characterized by a ¹⁹F NMR spectrum having a signal at two or more ppm values chosen from −112.6±0.2 ppm, −114.8±0.2 ppm, and −116.8±0.2 ppm.
   53. Citric acid cocrystal Form A of Compound I according to embodiment 32, characterized by a ¹⁹F NMR spectrum having signals at −112.6±0.2 ppm, −114.8±0.2 ppm, and −116.8±0.2 ppm.
   54. Citric acid cocrystal Form A of Compound I according to embodiment 32, characterized by a ¹⁹F NMR spectrum substantially similar to that in FIG. 9.
   55. Citric acid cocrystal Form A of Compound I according to embodiment 32, characterized by a DSC substantially similar to that in FIG. 11.
   56. Citric acid cocrystal Form A of Compound I according to embodiment 32, characterized by a DSC having an endotherm at 189° C.
   57. Citric acid cocrystal Form A of Compound I according to embodiment 32, characterized a TGA substantially similar to that in FIG. 10.
   58. Citric acid cocrystal Form A of Compound I according to embodiment 32, characterized a TGA showing negligible weight loss from ambient temperature until thermal degradation.
   59. A pharmaceutical composition comprising citric acid cocrystal Form A of Compound I according to any one of embodiments 32 to 58 and a pharmaceutically acceptable carrier.
   60. A method of treating APOL1 mediated kidney disease comprising administering to a patient in need thereof citric acid cocrystal Form A of Compound I according to any one of embodiments 32 to 58 or a pharmaceutical composition according to embodiment 59.
   61. The method according to embodiment 60, wherein the APOL1 mediated kidney disease is chosen from ESKD, NDKD, FSGS, HIV-associated nephropathy, arterionephrosclerosis, lupus nephritis, microalbuminuria, and chronic kidney disease.
   62. The method according to embodiment 60, wherein the APOL1 mediated kidney disease is chosen from ESKD, NDKD, and FSGS.
   63. The method according to any one of embodiments 60-62, wherein the APOL1 mediated kidney disease is associated with APOL1 genetic alleles chosen from homozygous G1: S342G:I384M and homozygous G2: N388del:Y389del.
   64. The method according to any one of embodiments 60-62, wherein the APOL1 mediated kidney disease is associated with compound heterozygous G1: S342G:I384M and G2: N388del:Y389del APOL1 genetic alleles.
   65. A method of inhibiting APOL1 activity comprising contacting said APOL1 with at least one entity according to any one of embodiments 32 to 58 or a pharmaceutical composition according to embodiment 59.
   66. The method according to embodiment 65, wherein the APOL1 is associated with APOL1 genetic alleles chosen from homozygous G: S342G:I384M and homozygous G2: N388del:Y389del.
   67. The method according to embodiment 65, wherein the APOL1 is associated with compound heterozygous G1: S342G:I384M and G2: N388del:Y389del APOL1 genetic alleles.
   68. Use of citric acid cocrystal Form A of Compound I according to any one of embodiments 32 to 58 in the manufacture of a medicament for treating APOL1 mediated kidney disease.
   69. Citric acid cocrystal Form A of Compound I according to any one of embodiments 32-58 for use in treating APOL1 mediated kidney disease.
   70. A method of preparing citric acid cocrystal Form A of Compound I comprising
mixing Compound I Form A with citric acid,
dissolving the mixture in 2 butanone (MEK),
stirring for 30 min-1 hour to form a slurry; and
centrifuging and then drying the solid at 55° C. overnight with nitrogen bleed.
   71. Piperazine cocrystal Form A cocrystal of Compound I.
   72. Piperazine cocrystal Form A of Compound I according to embodiment 71, characterized by an X-ray powder diffractogram having a signal at one or more two-theta values selected from 19.7±0.2, 17.3±0.2, 13.1±0.2, and 10.0±0.2.
   73. Piperazine cocrystal Form A of Compound I according to embodiment 71, characterized by an X-ray powder diffractogram having a signal at two or more two-theta values selected from 19.7±0.2, 17.3±0.2, 13.1±0.2, and 10.0±0.2.
   74. Piperazine cocrystal Form A of Compound I according to embodiment 71, characterized by an X-ray powder diffractogram having a signal at three or more two-theta values selected from 19.7±0.2, 17.3±0.2, 13.1±0.2, and 10.0±0.2.
   75. Piperazine cocrystal Form A of Compound I according to embodiment 71, characterized by an X-ray powder diffractogram having signals at 19.7±0.2, 17.3±0.2, 13.1±0.2, and 10.0±0.2 two-theta.
   76. Piperazine cocrystal Form A of Compound I according to embodiment 71, characterized by an X-ray powder diffractogram having (a) a signal at the following two-theta values 19.7±0.2, 17.3±0.2, 13.1±0.2, and 10.0±0.2; and (b) a signal at one or more two-theta values selected from 26.5±0.2, 22.2±0.2, 22.0±0.2, 16.9±0.2, 16.3±0.2, and 13.4±0.2.
   77. Piperazine cocrystal Form A of Compound I according to embodiment 71, characterized by an X-ray powder diffractogram having (a) a signal at the following two-theta values 19.7±0.2, 17.3±0.2, 13.1±0.2, and 10.0±0.2; and (b) a signal at two or more two-theta values selected from 26.5±0.2, 22.2±0.2, 22.0±0.2, 16.9±0.2, 16.3±0.2, and 13.4±0.2.
   78. Piperazine cocrystal Form A of Compound I according to embodiment 71, characterized by an X-ray powder diffractogram having (a) a signal at the following two-theta values 19.7±0.2, 17.3±0.2, 13.1±0.2, and 10.0±0.2; and (b) a signal at three or more two-theta values selected from 26.5±0.2, 22.2±0.2, 22.0±0.2, 16.9±0.2, 16.3±0.2, and 13.4±0.2
   79. Piperazine cocrystal Form A of Compound I according to embodiment 71, characterized by an X-ray powder diffractogram having a signals at 26.5±0.2, 22.2±0.2, 22.0±0.2, 19.7±0.2, 17.3±0.2, 16.9±0.2, 16.3±0.2, 13.4±0.2, 13.1±0.2, and 10.0±0.2 two-theta.
   80. Piperazine cocrystal Form A of Compound I according to embodiment 71, characterized by an X-ray powder diffractogram substantially similar to that in FIG. 12.
   81. Piperazine cocrystal Form A of Compound I according to embodiment 71, characterized by a ¹³C NMR spectrum having one or more signals selected from 111.0±0.2 ppm 72.8±0.2 ppm, 47.0±0.2 ppm, 45.1±0.2 ppm, and 44.8±0.2 ppm.
   82. Piperazine cocrystal Form A of Compound I according to embodiment 71, characterized by a ¹³C NMR spectrum having two or more signals selected from 111.0±0.2 ppm 72.8±0.2 ppm, 47.0±0.2 ppm, 45.1±0.2 ppm, and 44.8±0.2 ppm.
   83. Piperazine cocrystal Form A of Compound I according to embodiment 71, characterized by a ¹³C NMR spectrum having three or more signals selected from 111.0±0.2 ppm 72.8±0.2 ppm, 47.0±0.2 ppm, 45.1±0.2 ppm, and 44.8±0.2 ppm.
   84. Piperazine cocrystal Form A of Compound I according to embodiment 71, characterized by a ¹³C NMR spectrum signals at 111.0±0.2 ppm 72.8±0.2 ppm, 47.0±0.2 ppm, 45.1±0.2 ppm, and 44.8±0.2 ppm.
   85. Piperazine cocrystal Form A of Compound I according to embodiment 71, characterized by a ¹³C NMR spectrum having (a) signals at 111.0±0.2 ppm 72.8±0.2 ppm, 47.0±0.2 ppm, 45.1±0.2 ppm, and 44.8±0.2 ppm and (b) one or more signals selected from 130.5±0.2 ppm, 129.2±0.2 ppm, 129.0±0.2 ppm, 120.5±0.2 ppm, 119.9±0.2 ppm, 111.6±0.2 ppm, and 46.2±0.2 ppm.
   86. Piperazine cocrystal Form A of Compound I according to embodiment 71, characterized by a ¹³C NMR spectrum having (a) signals at 111.0±0.2 ppm 72.8±0.2 ppm, 47.0±0.2 ppm, 45.1±0.2 ppm, and 44.8±0.2 ppm and (b) two or more signals selected from 130.5±0.2 ppm, 129.2±0.2 ppm, 129.0±0.2 ppm, 120.5±0.2 ppm, 119.9±0.2 ppm, 111.6±0.2 ppm, and 46.2±0.2 ppm.
   87. Piperazine cocrystal Form A of Compound I according to embodiment 71, characterized by a ¹³C NMR spectrum having (a) signals at 111.0±0.2 ppm 72.8±0.2 ppm, 47.0±0.2 ppm, 45.1±0.2 ppm, and 44.8±0.2 ppm and (b) three or more signals selected from 130.5±0.2 ppm, 129.2±0.2 ppm, 129.0±0.2 ppm, 120.5±0.2 ppm, 119.9±0.2 ppm, 111.6±0.2 ppm, and 46.2±0.2 ppm.
   88. Piperazine cocrystal Form A of Compound I according to embodiment 71, characterized by a ¹³C NMR spectrum having signals at 130.5±0.2 ppm, 129.2±0.2 ppm, 120.5±0.2 ppm, 119.9±0.2 ppm, 111.6±0.2 ppm, 111.0±0.2 ppm, 72.8±0.2 ppm, 47.0±0.2 ppm, 46.2±0.2 ppm 45.1±0.2 ppm, and 44.8±0.2 ppm.
   89. Piperazine cocrystal Form A of Compound I according to embodiment 71, characterized by a ¹³C NMR spectrum substantially similar to that in FIG. 13.
   90. Piperazine cocrystal Form A of Compound I according to embodiment 71, characterized by a ¹⁹F NMR spectrum having a signal at −112.1±0.2 ppm.
   91. Piperazine cocrystal Form A of Compound I according to embodiment 71, characterized by a ¹⁹F NMR spectrum substantially similar to that in FIG. 14.
   92. Piperazine cocrystal Form A of Compound I according to embodiment 71, characterized by a DSC substantially similar to that in FIG. 16.
   93. Piperazine cocrystal Form A of Compound I according to embodiment 71, characterized by a DSC having showing multiple endothermic peaks at about 123° C. and 130° C.
   94. Piperazine cocrystal Form A of Compound I according to embodiment 71, characterized a TGA substantially similar to that in FIG. 15.
   95. Piperazine cocrystal Form A of Compound I according to embodiment 71, characterized a TGA showing about a 15% weight loss from ambient temperature to about 115° C. with continued weight loss to about 300° C.
   96. A pharmaceutical composition comprising piperazine cocrystal Form A of Compound I according to any one of embodiments 71 to 95 and a pharmaceutically acceptable carrier.
   97. A method of treating APOL1 mediated kidney disease comprising administering to a patient in need thereof piperazine cocrystal Form A of Compound I according to any one of embodiments 71 to 95 or a pharmaceutical composition according to embodiment 96.
   98. The method according to embodiment 97, wherein the APOL1 mediated kidney disease is chosen from ESKD, NDKD, FSGS, HIV-associated nephropathy, arterionephrosclerosis, lupus nephritis, microalbuminuria, and chronic kidney disease.
   99. The method according to embodiment 97, wherein the APOL1 mediated kidney disease is chosen from ESKD, NDKD, and FSGS.
   100. The method according to any one of embodiments 97-99, wherein the APOL1 mediated kidney disease is associated with APOL1 genetic alleles chosen from homozygous G1: S342G:I384M and homozygous G2: N388del:Y389del.
   101. The method according to any one of embodiments 97-99, wherein the APOL1 mediated kidney disease is associated with compound heterozygous G1: S342G:I384M and G2: N388del:Y389del APOL1 genetic alleles.
   102. A method of inhibiting APOL1 activity comprising contacting said APOL1 with at least one entity according to any one of embodiments 71 to 95 or a pharmaceutical composition according to embodiment 96.
   103. The method according to embodiment 102, wherein the APOL1 is associated with APOL1 genetic alleles chosen from homozygous G: S342G:I384M and homozygous G2: N388del:Y389del.
   104. The method according to embodiment 102, wherein the APOL1 is associated with compound heterozygous G1: S342G:I384M and G2: N388del:Y389del APOL1 genetic alleles.
   105. Use of piperazine cocrystal Form A of Compound I according to any one of embodiments 71 to 95 in the manufacture of a medicament for treating APOL1 mediated kidney disease.
   106. Piperazine cocrystal Form A of Compound I according to any one of embodiments 71-95 or the pharmaceutical composition according to embodiment 96, for use in treating APOL1 mediated kidney disease.
   107. A method of preparing piperazine cocrystal Form A of Compound I comprising
mixing Compound I Form A with piperazine and ethyl acetate,
sonicating mixture for about 30 minutes at ambient temperature,
isolating the solid material (piperazine cocrystal Form A).
   108. Urea cocrystal Form A cocrystal of Compound I.
   109. Urea cocrystal Form A of Compound I according to embodiment 108, characterized by an X-ray powder diffractogram having a signal at one or more two-theta values selected from 22.4±0.2, 21.2±0.2, 20.4±0.2, and 18.4±0.2.
   110. Urea cocrystal Form A of Compound I according to embodiment 108, characterized by an X-ray powder diffractogram having a signal at two or more two-theta values selected from 22.4±0.2, 21.2±0.2, 20.4±0.2, and 18.4±0.2.
   111. Urea cocrystal Form A of Compound I according to embodiment 108, characterized by an X-ray powder diffractogram having a signal at three or more two-theta values selected from 22.4±0.2, 21.2±0.2, 20.4±0.2, and 18.4±0.2.
   112. Urea cocrystal Form A of Compound I according to embodiment 108, characterized by an X-ray powder diffractogram having signals at 22.4±0.2, 21.2±0.2, 20.4±0.2, and 18.4±0.2 two-theta.
   113. Urea cocrystal Form A of Compound I according to embodiment 108, characterized by an X-ray powder diffractogram having (a) a signal at the following two-theta values 22.4±0.2, 21.2±0.2, 20.4±0.2, and 18.4±0.2; and (b) a signal at one or more two-theta values selected from 23.3±0.2, 21.7±0.2, 21.4±0.2, 21.3±0.2, 20.3±0.2, and 9.4±0.2.
   114. Urea cocrystal Form A of Compound I according to embodiment 108, characterized by an X-ray powder diffractogram having (a) a signal at the following two-theta values 22.4±0.2, 21.2±0.2, 20.4±0.2, and 18.4±0.2; and (b) a signal at two or more two-theta values selected from 23.3±0.2, 21.7±0.2, 21.4±0.2, 21.3±0.2, 20.3±0.2, and 9.4±0.2.
   115. Urea cocrystal Form A of Compound I according to embodiment 108, characterized by an X-ray powder diffractogram having (a) a signal at the following two-theta values 22.4±0.2, 21.2±0.2, 20.4±0.2, and 18.4±0.2; and (b) a signal at three or more two-theta values selected from 23.3±0.2, 21.7±0.2, 21.4±0.2, 21.3±0.2, 20.3±0.2, and 9.4±0.2.
   116. Urea cocrystal Form A of Compound I according to embodiment 108, characterized by an X-ray powder diffractogram having a signals at 23.3±0.2, 22.4±0.2, 21.7±0.2, 21.4±0.2, 21.3±0.2, 21.2±0.2, 20.4±0.2, 20.3±0.2, 18.4±0.2, and 9.4±0.2 two-theta.
   117. Urea cocrystal Form A of Compound I according to embodiment 108, characterized by an X-ray powder diffractogram substantially similar to that in FIG. 17.
   118. Urea cocrystal Form A of Compound I according to embodiment 108, characterized by a ¹³C NMR spectrum having one or more signals selected from 129.2±0.2 ppm, 120.3±0.2 ppm, 74.6±0.2 ppm, 58.4±0.2 ppm, and 44.6±0.2 ppm.
   119. Urea cocrystal Form A of Compound I according to embodiment 108, characterized by a ¹³C NMR spectrum having two or more signals selected from 129.2±0.2 ppm, 120.3±0.2 ppm, 74.6±0.2 ppm, 58.4±0.2 ppm, and 44.6±0.2 ppm.
   120. Urea cocrystal Form A of Compound I according to embodiment 108, characterized by a ¹³C NMR spectrum having three or more signals selected from 129.2±0.2 ppm, 120.3±0.2 ppm, 74.6±0.2 ppm, 58.4±0.2 ppm, and 44.6±0.2 ppm.
   121. Urea cocrystal Form A of Compound I according to embodiment 108, characterized by a ¹³C NMR spectrum signals at 129.2±0.2 ppm, 120.3±0.2 ppm, 74.6±0.2 ppm, 58.4±0.2 ppm, and 44.6±0.2 ppm.
   122. Urea cocrystal Form A of Compound I according to embodiment 108, characterized by a ¹³C NMR spectrum having (a) signals at 129.2±0.2 ppm, 120.3±0.2 ppm, 74.6±0.2 ppm, 58.4±0.2 ppm, and 44.6±0.2 ppm; and (b) one or more signals selected from 175.4±0.2 ppm, 175.0±0.2 ppm, 135.5±0.2 ppm, 38.4±0.2 ppm, and 18.9±0.2 ppm.
   123. Urea cocrystal Form A of Compound I according to embodiment 108, characterized by a ¹³C NMR spectrum having (a) signals at 129.2±0.2 ppm, 120.3±0.2 ppm, 74.6±0.2 ppm, 58.4±0.2 ppm, and 44.6±0.2 ppm; and (b) two or more signals selected from 175.4±0.2 ppm, 175.0±0.2 ppm, 135.5±0.2 ppm, 38.4±0.2 ppm, and 18.9±0.2 ppm.
   124. Urea cocrystal Form A of Compound I according to embodiment 108, characterized by a ¹³C NMR spectrum having (a) signals at 129.2±0.2 ppm, 120.3±0.2 ppm, 74.6±0.2 ppm, 58.4±0.2 ppm, and 44.6±0.2 ppm; and (b) three or more signals selected from 175.4±0.2 ppm, 175.0±0.2 ppm, 135.5±0.2 ppm, 38.4±0.2 ppm, and 18.9±0.2 ppm.
   125. Urea cocrystal Form A of Compound I according to embodiment 108, characterized by a ¹³C NMR spectrum having signals at 175.4±0.2 ppm, 175.0±0.2 ppm, 135.5±0.2 ppm, 129.2±0.2 ppm, 120.3±0.2 ppm, 74.6±0.2 ppm, 58.4±0.2 ppm, and 44.6±0.2 ppm, 38.4±0.2 ppm, and 18.9±0.2 ppm.
   126. Urea cocrystal Form A of Compound I according to embodiment 108, characterized by a ¹³C NMR spectrum substantially similar to that in FIG. 18.
   127. Urea cocrystal Form A of Compound I according to embodiment 108, characterized by a ¹⁹F NMR spectrum having a signal at one or more ppm values chosen from −110.8±0.2 ppm, −113.2±0.2 ppm, and −113.7±0.2 ppm.
   128. Urea cocrystal Form A of Compound I according to embodiment 108, characterized by a ¹⁹F NMR spectrum having a signal at two or more ppm values chosen from −110.8±0.2 ppm, −113.2±0.2 ppm, and −113.7±0.2 ppm.
   129. Urea cocrystal Form A of Compound I according to embodiment 108, characterized by a ¹⁹F NMR spectrum having signals at −110.8±0.2 ppm, −113.2±0.2 ppm, and −113.7±0.2 ppm.
   130. Urea cocrystal Form A of Compound I according to embodiment 108, characterized by a ¹⁹F NMR spectrum substantially similar to that in FIG. 19.
   131. Urea cocrystal Form A of Compound I according to embodiment 108, characterized by a DSC substantially similar to that in FIG. 21.
   132. Urea cocrystal Form A of Compound I according to embodiment 108, characterized by a DSC having an endothermic peak at about 182° C.
   133. Urea cocrystal Form A of Compound I according to embodiment 108, characterized a TGA substantially similar to that in FIG. 20.
   134. Urea cocrystal Form A of Compound I according to embodiment 108, characterized a TGA showing gradual weight loss of about 0.3% from ambient temperature to thermal degradation.
   135. A pharmaceutical composition comprising urea cocrystal Form A of Compound I according to any one of embodiments 108 to 134 and a pharmaceutically acceptable carrier.
   136. A method of treating APOL1 mediated kidney disease comprising administering to a patient in need thereof urea cocrystal Form A of Compound I according to any one of embodiments 108 to 134 or a pharmaceutical composition according to embodiment 135.
   137. The method according to embodiment 136, wherein the APOL1 mediated kidney disease is chosen from ESKD, NDKD, FSGS, HIV-associated nephropathy, arterionephrosclerosis, lupus nephritis, microalbuminuria, and chronic kidney disease.
   138. The method according to embodiment 136, wherein the APOL1 mediated kidney disease is chosen from ESKD, NDKD, and FSGS.
   139. The method according to any one of embodiments 136-138, wherein the APOL1 mediated kidney disease is associated with APOL1 genetic alleles chosen from homozygous G1: S342G:I384M and homozygous G2: N388del:Y389del.
   140. The method according to any one of embodiments 136-138, wherein the APOL1 mediated kidney disease is associated with compound heterozygous G1: S342G:I384M and G2: N388del:Y389del APOL1 genetic alleles.
   141. A method of inhibiting APOL1 activity comprising contacting said APOL1 with at least one entity according to any one of embodiments 108 to 134 or a pharmaceutical composition according to embodiment 135.
   142. The method according to embodiment 141, wherein the APOL1 is associated with APOL1 genetic alleles chosen from homozygous G: S342G:I384M and homozygous G2: N388del:Y389del.
   143. The method according to embodiment 141, wherein the APOL1 is associated with compound heterozygous G1: S342G:I384M and G2: N388del:Y389del APOL1 genetic alleles.
   144. Use of urea cocrystal Form A of Compound I according to any one of embodiments 108 to 134 in the manufacture of a medicament for treating APOL1 mediated kidney disease.
   145. Urea cocrystal Form A of Compound I according to any one of embodiments 108 to 134 or the pharmaceutical composition according to embodiment 135 for use in treating APOL1 mediated kidney disease.
   146. A method of preparing urea cocrystal Form A of Compound I comprising dissolving Compound I Form A in solvent and adding urea, stirring for 1 hour at ambient temperature to form pre-saturated solution; adding a preground mixture of Compound I Form A and dry urea to make a slurry; heating to 25° C. and stirring for about 24 hours. isolating Compound I urea cocrystal Form A.
   147. Nicotinamide cocrystal Form A cocrystal of Compound I.
   148. Nicotinamide cocrystal Form A of Compound I according to embodiment 147, characterized by an X-ray powder diffractogram having a signal at one or more two-theta values selected from 18.3±0.2, 15.3±0.2, 6.3±0.2, and 5.1±0.2.
   149. Nicotinamide cocrystal Form A of Compound I according to embodiment 147, characterized by an X-ray powder diffractogram having a signal at two or more two-theta values selected from 18.3±0.2, 15.3±0.2, 6.3±0.2, and 5.1±0.2.
   150. Nicotinamide cocrystal Form A of Compound I according to embodiment 147, characterized by an X-ray powder diffractogram having a signal at three or more two-theta values selected from 18.3±0.2, 15.3±0.2, 6.3±0.2, and 5.1±0.2.
   151. Nicotinamide cocrystal Form A of Compound I according to embodiment 147, characterized by an X-ray powder diffractogram having signals at 18.3±0.2, 15.3±0.2, 6.3±0.2, and 5.1±0.2.
   152. Nicotinamide cocrystal Form A of Compound I according to embodiment 147, characterized by an X-ray powder diffractogram having (a) a signal at one or more two-theta values selected from 18.3±0.2, 15.3±0.2, 6.3±0.2, and 5.1±0.2; and (b) a signal at 19.6±0.2 degrees two-theta.
   153. Nicotinamide cocrystal Form A of Compound I according to embodiment 147, characterized by an X-ray powder diffractogram having (a) a signal at two or more two-theta values selected from 18.3±0.2, 15.3±0.2, 6.3±0.2, and 5.1±0.2; and (b) a signal at 19.6±0.2 degrees two-theta.
   154. Nicotinamide cocrystal Form A of Compound I according to embodiment 147, characterized by an X-ray powder diffractogram having (a) a signal at three or more two-theta values selected from 18.3±0.2, 15.3±0.2, 6.3±0.2, and 5.1±0.2; and (b) a signal at 19.6±0.2 degrees two-theta.
   155. Nicotinamide cocrystal Form A of Compound I according to embodiment 147, characterized by an X-ray powder diffractogram having signals at 19.6±0.2, 18.3±0.2, 15.3±0.2, 6.3±0.2, and 5.1±0.2.
   156. Nicotinamide cocrystal Form A of Compound I according to embodiment 147, characterized by an X-ray powder diffractogram substantially similar to that in FIG. 22.
   157. Nicotinamide cocrystal Form A of Compound I according to embodiment 147, characterized by a ¹³C NMR spectrum having one or more signals selected from 149.2±0.2 ppm, 136.1±0.2 ppm, 128.3±0.2 ppm, 112.0±0.2 ppm, and 71.4±0.2 ppm.
   158. Nicotinamide cocrystal Form A of Compound I according to embodiment 147, characterized by a ¹³C NMR spectrum having two or more signals selected from 149.2±0.2 ppm, 136.1±0.2 ppm, 128.3±0.2 ppm, 112.0±0.2 ppm, and 71.4±0.2 ppm.
   159. Nicotinamide cocrystal Form A of Compound I according to embodiment 147, characterized by a ¹³C NMR spectrum having three or more signals selected from 149.2±0.2 ppm, 136.1±0.2 ppm, 128.3±0.2 ppm, 112.0±0.2 ppm, and 71.4±0.2 ppm.
   160. Nicotinamide cocrystal Form A of Compound I according to embodiment 147, characterized by a ¹³C NMR spectrum signals at 149.2±0.2 ppm, 136.1±0.2 ppm, 128.3±0.2 ppm, 112.0±0.2 ppm, and 71.4±0.2 ppm.
   161. Nicotinamide cocrystal Form A of Compound I according to embodiment 147, characterized by a ¹³C NMR spectrum having (a) signals at 149.2±0.2 ppm, 136.1±0.2 ppm, 128.3±0.2 ppm, 112.0±0.2 ppm, and 71.4±0.2 ppm; and (b) one or more signals selected from 174.5±0.2 ppm, 129.0±0.2 ppm, 121.2±0.2 ppm, 119.2±0.2 ppm, and 112.7±0.2 ppm.
   162. Nicotinamide cocrystal Form A of Compound I according to embodiment 147, characterized by a ¹³C NMR spectrum having (a) signals at 149.2±0.2 ppm, 136.1±0.2 ppm, 128.3±0.2 ppm, 112.0±0.2 ppm, and 71.4±0.2 ppm; and (b) two or more signals selected from 174.5±0.2 ppm, 129.0±0.2 ppm, 121.2±0.2 ppm, 119.2±0.2 ppm, and 112.7±0.2 ppm.
   163. Nicotinamide cocrystal Form A of Compound I according to embodiment 147, characterized by a ¹³C NMR spectrum having (a) signals at 149.2±0.2 ppm, 136.1±0.2 ppm, 128.3±0.2 ppm, 112.0±0.2 ppm, and 71.4±0.2 ppm; and (b) three or more signals selected from 174.5±0.2 ppm, 129.0±0.2 ppm, 121.2±0.2 ppm, 119.2±0.2 ppm, and 112.7±0.2 ppm.
   164. Nicotinamide cocrystal Form A of Compound I according to embodiment 147, characterized by a ¹³C NMR spectrum having signals at 174.5±0.2 ppm, 149.2±0.2 ppm, 136.1±0.2 ppm, 129.0±0.2 ppm, 128.3±0.2 ppm, 121.2±0.2 ppm, 119.2±0.2 ppm, 112.7±0.2 ppm, 112.0±0.2 ppm, and 71.4±0.2 ppm.
   165. Nicotinamide cocrystal Form A of Compound I according to embodiment 147, characterized by a ¹³C NMR spectrum substantially similar to that in FIG. 23.
   166. Nicotinamide cocrystal Form A of Compound I according to embodiment 147, characterized by a ¹⁹F NMR spectrum having a signal at one or more ppm values chosen from −116.4±0.2 ppm, −117.9±0.2 ppm, and −118.5±0.2 ppm.
   167. Nicotinamide cocrystal Form A of Compound I according to embodiment 147, characterized by a ¹⁹F NMR spectrum having a signal at two or more ppm values chosen from −116.4±0.2 ppm, −117.9±0.2 ppm, and −118.5±0.2 ppm.
   168. Nicotinamide cocrystal Form A of Compound I according to embodiment 147, characterized by a ¹⁹F NMR spectrum having signals at −116.4±0.2 ppm, −117.9±0.2 ppm, and −118.5±0.2 ppm.
   169. Nicotinamide cocrystal Form A of Compound I according to embodiment 147, characterized by a ¹⁹F NMR spectrum substantially similar to that in FIG. 24.
   170. Nicotinamide cocrystal Form A of Compound I according to embodiment 147, characterized by a DSC substantially similar to that in FIG. 26.
   171. Nicotinamide cocrystal Form A of Compound I according to embodiment 147, characterized by a DSC having an endothermic peak at about 89° C.
   172. Nicotinamide cocrystal Form A of Compound I according to embodiment 147, characterized a TGA substantially similar to that in FIG. 25.
   173. Nicotinamide cocrystal Form A of Compound I according to embodiment 147, characterized a TGA showing weight loss of about 7% from ambient temperature to 125° C.
   174. A pharmaceutical composition comprising nicotinamide cocrystal Form A of Compound I according to any one of embodiments 147 to 173 and a pharmaceutically acceptable carrier.
   175. A method of treating APOL1 mediated kidney disease comprising administering to a patient in need thereof nicotinamide cocrystal Form A of Compound I according to any one of embodiments 147 to 173 or a pharmaceutical composition according to embodiment 174.
   176. The method according to embodiment 175, wherein the APOL1 mediated kidney disease is chosen from ESKD, NDKD, FSGS, HIV-associated nephropathy, arterionephrosclerosis, lupus nephritis, microalbuminuria, and chronic kidney disease.
   177. The method according to embodiment 175, wherein the APOL1 mediated kidney disease is chosen from ESKD, NDKD, and FSGS.
   178. The method according to any one of embodiments 175-177, wherein the APOL1 mediated kidney disease is associated with APOL1 genetic alleles chosen from homozygous G1: S342G:I384M and homozygous G2: N388del:Y389del.
   179. The method according to any one of embodiments 175-177, wherein the APOL1 mediated kidney disease is associated with compound heterozygous G1: S342G:I384M and G2: N388del:Y389del APOL1 genetic alleles.
   180. A method of inhibiting APOL1 activity comprising contacting said APOL1 with at least one entity according to any one of embodiments 147 to 173 or a pharmaceutical composition according to embodiment 174.
   181. The method according to embodiment 180, wherein the APOL1 is associated with APOL1 genetic alleles chosen from homozygous G: S342G:I384M and homozygous G2: N388del:Y389del.
   182. The method according to embodiment 180, wherein the APOL1 is associated with compound heterozygous G1: S342G:I384M and G2: N388del:Y389del APOL1 genetic alleles.
   183. Use of nicotinamide cocrystal Form A of Compound I according to any one of embodiments 147 to 173 in the manufacture of a medicament for treating APOL1 mediated kidney disease.
   184. Nicotinamide cocrystal Form A of Compound I according to any one of embodiments 147-173 or the pharmaceutical composition of embodiment 174 for use in treating APOL1 mediated kidney disease.
   185. A method of preparing nicotinamide cocrystal Form A of Compound I comprising
dissolving Compound I Form A in solvent and adding nicotinamide,
stirring for 1 hour at ambient temperature to form pre-saturated solution;
adding a preground mixture of Compound I Form A and dry nicotinamide to make a slurry;
heating to 25° C. and stirring for about 24 hours; and
isolating Compound I nicotinamide cocrystal Form A.
   186. Nicotinamide cocrystal Form B cocrystal of Compound I.
   187. Nicotinamide cocrystal Form B of Compound I according to embodiment 186, characterized by an X-ray powder diffractogram having a signal at one or more two-theta values selected from 20.0±0.2, 15.1±0.2, 5.0±0.2, and 4.9±0.2.
   188. Nicotinamide cocrystal Form B of Compound I according to embodiment 186, characterized by an X-ray powder diffractogram having a signal at two or more two-theta values selected from 20.0±0.2, 15.1±0.2, 5.0±0.2, and 4.9±0.2.
   189. Nicotinamide cocrystal Form B of Compound I according to embodiment 186, characterized by an X-ray powder diffractogram having a signal at three or more two-theta values selected from 20.0±0.2, 15.1±0.2, 5.0±0.2, and 4.9±0.2.
   190. Nicotinamide cocrystal Form B of Compound I according to embodiment 186, characterized by an X-ray powder diffractogram having signals at 20.0±0.2, 15.1±0.2, 5.0±0.2, and 4.9±0.2 two-theta.
   191. Nicotinamide cocrystal Form B of Compound I according to embodiment 186, characterized by an X-ray powder diffractogram having (a) a signal at the following two-theta values 20.0±0.2, 15.1±0.2, 5.0±0.2, and 4.9±0.2; and (b) a signal at one or more two-theta values selected from 19.2±0.2, 18.0±0.2, 16.5±0.2, and 6.6±0.2.
   192. Nicotinamide cocrystal Form B of Compound I according to embodiment 186, characterized by an X-ray powder diffractogram having (a) a signal at the following two-theta values 20.0±0.2, 15.1±0.2, 5.0±0.2, and 4.9±0.2; and (b) a signal at two or more two-theta values selected from 19.2±0.2, 18.0±0.2, 16.5±0.2, and 6.6±0.2.
   193. Nicotinamide cocrystal Form B of Compound I according to embodiment 186, characterized by an X-ray powder diffractogram having (a) a signal at the following two-theta values 20.0±0.2, 15.1±0.2, 5.0±0.2, and 4.9±0.2; and (b) a signal at three or more two-theta values selected from 19.2±0.2, 18.0±0.2, 16.5±0.2, and 6.6±0.2.
   194. Nicotinamide cocrystal Form B of Compound I according to embodiment 186, characterized by an X-ray powder diffractogram having signals at 20.0±0.2, 19.2±0.2, 18.0±0.2, 16.5±0.2, 15.1±0.2, 6.6±0.2, 5.0±0.2, and 4.9±0.2 two-theta.
   195. Nicotinamide cocrystal Form B of Compound I according to embodiment 186, characterized by an X-ray powder diffractogram substantially similar to that in FIG. 27.
   196. Nicotinamide cocrystal Form B of Compound I according to embodiment 186, characterized by a ¹³C NMR spectrum having one or more signals selected from 136.4±0.2 ppm, 128.9±0.2 ppm, 121.7±0.2 ppm, 119.2±0.2 ppm, and 111.6±0.2 ppm.
   197. Nicotinamide cocrystal Form B of Compound I according to embodiment 186, characterized by a ¹³C NMR spectrum having two or more signals selected from 136.4±0.2 ppm, 128.9±0.2 ppm, 121.7±0.2 ppm, 119.2±0.2 ppm, and 111.6±0.2 ppm.
   198. Nicotinamide cocrystal Form B of Compound I according to embodiment 186, characterized by a ¹³C NMR spectrum having three or more signals selected from 136.4±0.2 ppm, 128.9±0.2 ppm, 121.7±0.2 ppm, 119.2±0.2 ppm, and 111.6±0.2 ppm.
   199. Nicotinamide cocrystal Form B of Compound I according to embodiment 186, characterized by a ¹³C NMR spectrum signals at 136.4±0.2 ppm, 128.9±0.2 ppm, 121.7±0.2 ppm, 119.2±0.2 ppm, and 111.6±0.2 ppm.
   200. Nicotinamide cocrystal Form B of Compound I according to embodiment 186, characterized by a ¹³C NMR spectrum having (a) signals at 136.4±0.2 ppm, 128.9±0.2 ppm, 121.7±0.2 ppm, 119.2±0.2 ppm, and 111.6±0.2 ppm; and (b) one or more signals selected from 174.5±0.2 ppm, 120.6±0.2 ppm, 120.2±0.2 ppm, 62.8±0.2 ppm, and 18.1±0.2 ppm.
   201. Nicotinamide cocrystal Form B of Compound I according to embodiment 186, characterized by a ¹³C NMR spectrum having (a) signals at 136.4±0.2 ppm, 128.9±0.2 ppm, 121.7±0.2 ppm, 119.2±0.2 ppm, and 111.6±0.2 ppm; and (b) two or more signals selected from 174.5±0.2 ppm, 120.6±0.2 ppm, 120.2±0.2 ppm, 62.8±0.2 ppm, and 18.1±0.2 ppm.
   202. Nicotinamide cocrystal Form B of Compound I according to embodiment 186, characterized by a ¹³C NMR spectrum having (a) signals at 136.4±0.2 ppm, 128.9±0.2 ppm, 121.7±0.2 ppm, 119.2±0.2 ppm, and 111.6±0.2 ppm; and (b) three or more signals selected from 174.5±0.2 ppm, 120.6±0.2 ppm, 120.2±0.2 ppm, 62.8±0.2 ppm, and 18.1±0.2 ppm.
   203. Nicotinamide cocrystal Form B of Compound I according to embodiment 186, characterized by a ¹³C NMR spectrum having signals at 174.5±0.2 ppm, 136.4±0.2 ppm, 128.9±0.2 ppm, 121.7±0.2 ppm, 120.6±0.2 ppm, 120.2±0.2 ppm, 119.2±0.2 ppm, 111.6±0.2 ppm 62.8±0.2 ppm, and 18.1±0.2 ppm.
   204. Nicotinamide cocrystal Form B of Compound I according to embodiment 186, characterized by a ¹³C NMR spectrum substantially similar to that in FIG. 28.
   205. Nicotinamide cocrystal Form B of Compound I according to embodiment 186, characterized by a ¹⁹F NMR spectrum having a signal at one or more ppm values chosen from −111.0±0.2 ppm, −113.0±0.2 ppm, and −115.4±0.2 ppm.
   206. Nicotinamide cocrystal Form B of Compound I according to embodiment 186, characterized by a ¹⁹F NMR spectrum having a signal at two or more ppm values chosen from −111.0±0.2 ppm, −113.0±0.2 ppm, and −115.4±0.2 ppm.
   207. Nicotinamide cocrystal Form B of Compound I according to embodiment 186, characterized by a ¹⁹F NMR spectrum having signals at −111.0±0.2 ppm, −113.0±0.2 ppm, and −115.4±0.2 ppm.
   208. Nicotinamide cocrystal Form B of Compound I according to embodiment 186, characterized by a ¹⁹F NMR spectrum substantially similar to that in FIG. 29.
   209. A pharmaceutical composition comprising nicotinamide cocrystal Form B of Compound I according to any one of embodiments 186 to 208 and a pharmaceutically acceptable carrier.
   210. A method of treating APOL1 mediated kidney disease comprising administering to a patient in need thereof nicotinamide cocrystal Form B of Compound I according to any one of embodiments 186 to 208 or a pharmaceutical composition according to embodiment 209.
   211. The method according to embodiment 210, wherein the APOL1 mediated kidney disease is chosen from ESKD, NDKD, FSGS, HIV-associated nephropathy, arterionephrosclerosis, lupus nephritis, microalbuminuria, and chronic kidney disease.
   212. The method according to embodiment 210, wherein the APOL1 mediated kidney disease is chosen from ESKD, NDKD, and FSGS.
   213. The method according to any one of embodiments 210-212, wherein the APOL1 mediated kidney disease is associated with APOL1 genetic alleles chosen from homozygous G1: S342G:I384M and homozygous G2: N388del:Y389del.
   214. The method according to any one of embodiments 210-212, wherein the APOL1 mediated kidney disease is associated with compound heterozygous G1: S342G:I384M and G2: N388del:Y389del APOL1 genetic alleles.
   215. A method of inhibiting APOL1 activity comprising contacting said APOL1 with at least one entity according to any one of embodiments 186 to 208 or a pharmaceutical composition according to embodiment 209.
   216. The method according to embodiment 215, wherein the APOL1 mediated kidney disease is associated with compound heterozygous G1: S342G:I384M and G2: N388del:Y389del APOL1 genetic alleles.
   217. The method according to embodiment 215, wherein the APOL1 is associated with compound heterozygous G1: S342G:I384M and G2: N388del:Y389del APOL1 genetic alleles.
   218. Use of nicotinamide cocrystal Form B of Compound I according to any one of embodiments 186 to 208 in the manufacture of a medicament for treating APOL1 mediated kidney disease.
   219. Nicotinamide cocrystal Form B of Compound I according to any one of embodiments 186-208 or the pharmaceutical composition of embodiment 209 for use in treating APOL1 mediated kidney disease.
   220. A method of preparing nicotinamide cocrystal Form B of Compound I comprising
mixing Compound I Form A with nicotinamide (1:1) in a ball mill vessel with pentanol;
shaking at 15 hertz for about 30 minutes; and
isolating nicotinamide cocrystal Form B of Compound I.
   221. Aspartame cocrystal Form A cocrystal of Compound I.
   222. Aspartame cocrystal Form A of Compound I according to embodiment 221, characterized by an X-ray powder diffractogram having a signal at one or more two-theta values selected from 22.7±0.2, 21.2±0.2, 20.6±0.2, 20.3±0.2, and 6.9±0.2.
   223. Aspartame cocrystal Form A of Compound I according to embodiment 221, characterized by an X-ray powder diffractogram having a signal at two or more two-theta values selected from 22.7±0.2, 21.2±0.2, 20.6±0.2, 20.3±0.2, and 6.9±0.2.
   224. Aspartame cocrystal Form A of Compound I according to embodiment 221, characterized by an X-ray powder diffractogram having a signal at three or more two-theta values selected from 22.7±0.2, 21.2±0.2, 20.6±0.2, 20.3±0.2, and 6.9±0.2.
   225. Aspartame cocrystal Form A of Compound I according to embodiment 221, characterized by an X-ray powder diffractogram having signals at 22.7±0.2, 21.2±0.2, 20.6±0.2, 20.3±0.2, and 6.9±0.2 two-theta.
   226. Aspartame cocrystal Form A of Compound I according to embodiment 221, characterized by an X-ray powder diffractogram having (a) a signal at the following two-theta values 22.7±0.2, 21.2±0.2, 20.6±0.2, 20.3±0.2, and 6.9±0.2 and (b) a signal at one or more two-theta values selected from 24.0±0.2, 21.6±0.2, 18.5±0.2, 16.0±0.2, and 7.4±0.2.
   227. Aspartame cocrystal Form A of Compound I according to embodiment 221, characterized by an X-ray powder diffractogram having (a) a signal at the following two-theta values 22.7±0.2, 21.2±0.2, 20.6±0.2, 20.3±0.2, and 6.9±0.2; and (b) a signal at two or more two-theta values selected from 24.0±0.2, 21.6±0.2, 18.5±0.2, 16.0±0.2, and 7.4±0.2.
   228. Aspartame cocrystal Form A of Compound I according to embodiment 221, characterized by an X-ray powder diffractogram having (a) a signal at the following two-theta values 22.7±0.2, 21.2±0.2, 20.6±0.2, 20.3±0.2, and 6.9±0.2; and (b) a signal at three or more two-theta values selected from 24.0±0.2, 21.6±0.2, 18.5±0.2, 16.0±0.2, and 7.4±0.2.
   229. Aspartame cocrystal Form A of Compound I according to embodiment 221, characterized by an X-ray powder diffractogram having a signals at 24.0±0.2, 22.7±0.2, 21.6±0.2, 21.2±0.2, 20.6±0.2, 20.3±0.2, 18.5±0.2, 16.0±0.2, 7.4±0.2, and 6.9±0.2.
   230. Aspartame cocrystal Form A of Compound I according to embodiment 221, characterized by an X-ray powder diffractogram substantially similar to that in FIG. 30.
   231. Aspartame cocrystal Form A of Compound I according to embodiment 221, characterized by a DSC substantially similar to that in FIG. 32.
   232. Aspartame cocrystal Form A of Compound I according to embodiment 221, characterized by a DSC having an endothermic peak at about 147° C.
   233. Aspartame cocrystal Form A of Compound I according to embodiment 221, characterized a TGA substantially similar to that in FIG. 31.
   234. Aspartame cocrystal Form A of Compound I according to embodiment 221, characterized a TGA showing weight loss of about 10% from ambient temperature to about 144° C.
   235. A pharmaceutical composition comprising aspartame cocrystal Form A of Compound I according to any one of embodiments 221 to 234 and a pharmaceutically acceptable carrier.
   236. A method of treating APOL1 mediated kidney disease comprising administering to a patient in need thereof aspartame cocrystal Form A of Compound I according to any one of embodiments 221 to 234 or a pharmaceutical composition according to embodiment 235.
   237. The method according to embodiment 236, wherein the APOL1 mediated kidney disease is chosen from ESKD, NDKD, FSGS, HIV-associated nephropathy, arterionephrosclerosis, lupus nephritis, microalbuminuria, and chronic kidney disease.
   238. The method according to embodiment 236, wherein the APOL1 mediated kidney disease is chosen from ESKD, NDKD, and FSGS.
   239. The method according to any one of embodiments 236-238, wherein the APOL1 mediated kidney disease is associated with APOL1 genetic alleles chosen from homozygous G1: S342G:I384M and homozygous G2: N388del:Y389del.
   240. The method according to any one of embodiments 236-238, wherein the APOL1 mediated kidney disease is associated with compound heterozygous G1: S342G:I384M and G2: N388del:Y389del APOL1 genetic alleles.
   241. A method of inhibiting APOL1 activity comprising contacting said APOL1 with at least one entity according to any one of embodiments 221 to 234 or a pharmaceutical composition according to embodiment 235.
   242. The method according to embodiment 241, wherein the APOL1 is associated with APOL1 genetic alleles chosen from homozygous G: S342G:I384M and homozygous G2: N388del:Y389del.
   243. The method according to embodiment 241, wherein the APOL1 is associated with compound heterozygous G1: S342G:I384M and G2: N388del:Y389del APOL1 genetic alleles.
   244. Use of aspartame cocrystal Form A of Compound I according to any one of embodiments 221 to 234 in the manufacture of a medicament for treating APOL1 mediated kidney disease.
   245. Aspartame cocrystal Form A of Compound I according to any one of embodiments 221-234 or the pharmaceutical composition of embodiment 235 for use in treating APOL1 mediated kidney disease.
   246. A method of preparing aspartame cocrystal Form A of Compound I comprising
mixing Compound I Form A with aspartame in a ball mill vessel with pentanol;
shaking at 100 hertz for about 30 minutes;
isolating aspartame cocrystal Form A of Compound I.
   247. Glutaric acid cocrystal Form A cocrystal of Compound I.
   248. Glutaric acid cocrystal Form A of Compound I according to embodiment 247, characterized by an X-ray powder diffractogram having a signal at one or more two-theta values selected from 26.9±0.2, 22.2±0.2, 19.1±0.2, 18.9±0.2, and 9.4±0.2.
   249. Glutaric acid cocrystal Form A of Compound I according to embodiment 247, characterized by an X-ray powder diffractogram having a signal at two or more two-theta values selected from 26.9±0.2, 22.2±0.2, 19.1±0.2, 18.9±0.2, and 9.4±0.2.
   250. Glutaric acid cocrystal Form A of Compound I according to embodiment 247, characterized by an X-ray powder diffractogram having a signal at three or more two-theta values selected from 26.9±0.2, 22.2±0.2, 19.1±0.2, 18.9±0.2, and 9.4±0.2.
   251. Glutaric acid cocrystal Form A of Compound I according to embodiment 247, characterized by an X-ray powder diffractogram having signals at 26.9±0.2, 22.2±0.2, 19.1±0.2, 18.9±0.2, and 9.4±0.2 two-theta.
   252. Glutaric acid cocrystal Form A of Compound I according to embodiment 247, characterized by an X-ray powder diffractogram having (a) a signal at the following two-theta values 26.9±0.2, 22.2±0.2, 19.1±0.2, 18.9±0.2, and 9.4±0.2and (b) a signal at one or more two-theta values selected from 23.2±0.2, 21.9±0.2, 18.0±0.2, 13.5±0.2, and 11.0±0.2.
   253. Glutaric acid cocrystal Form A of Compound I according to embodiment 247, characterized by an X-ray powder diffractogram having (a) a signal at the following two-theta values 26.9±0.2, 22.2±0.2, 19.1±0.2, 18.9±0.2, and 9.4±0.2; and (b) a signal at two or more two-theta values selected from 23.2±0.2, 21.9±0.2, 18.0±0.2, 13.5±0.2, and 11.0±0.2.
   254. Glutaric acid cocrystal Form A of Compound I according to embodiment 247, characterized by an X-ray powder diffractogram having (a) a signal at the following two-theta values 26.9±0.2, 22.2±0.2, 19.1±0.2, 18.9±0.2, and 9.4±0.2; and (b) a signal at three or more two-theta values selected from 23.2±0.2, 21.9±0.2, 18.0±0.2, 13.5±0.2, and 11.0±0.2.
   255. Glutaric acid cocrystal Form A of Compound I according to embodiment 247, characterized by an X-ray powder diffractogram having signals at 26.9±0.2, 23.2±0.2, 22.2±0.2, 21.9±0.2, 19.1±0.2, 18.9±0.2, 18.0±0.2, 13.5±0.2, 11.0±0.2, and 9.4±0.22 two-theta.
   256. Glutaric acid cocrystal Form A of Compound I according to embodiment 247, characterized by an X-ray powder diffractogram substantially similar to that in FIG. 33.
   257. Glutaric acid cocrystal Form A of Compound I according to embodiment 247, characterized by a DSC substantially similar to that in FIG. 35.
   258. Glutaric acid cocrystal Form A of Compound I according to embodiment 247, characterized by a DSC having two endotherms at about 116° C. and about 227° C.
   259. Glutaric acid cocrystal Form A of Compound I according to embodiment 247, characterized a TGA substantially similar to that in FIG. 34.
   260. Glutaric acid cocrystal Form A of Compound I according to embodiment 247, characterized a TGA showing weight loss of about 5% from ambient temperature to about 188° C.
   261. A pharmaceutical composition comprising glutaric acid cocrystal Form A of Compound I according to any one of embodiments 247 to 260 and a pharmaceutically acceptable carrier.
   262. A method of treating APOL1 mediated kidney disease comprising administering to a patient in need thereof glutaric acid cocrystal Form A of Compound I according to any one of embodiments 247 to 260 or a pharmaceutical composition according to embodiment 261.
   263. The method according to embodiment 262, wherein the APOL1 mediated kidney disease is chosen from ESKD, NDKD, FSGS, HIV-associated nephropathy, arterionephrosclerosis, lupus nephritis, microalbuminuria, and chronic kidney disease.
   264. The method according to embodiment 262, wherein the APOL1 mediated kidney disease is chosen from ESKD, NDKD, and FSGS.
   265. The method according to any one of embodiments 262-264, wherein the APOL1 mediated kidney disease is associated with APOL1 genetic alleles chosen from homozygous G1: S342G:I384M and homozygous G2: N388del:Y389del.
   266. The method according to any one of embodiments 262-264, wherein the APOL1 mediated kidney disease is associated with compound heterozygous G1: S342G:I384M and G2: N388del:Y389del APOL1 genetic alleles.
   267. A method of inhibiting APOL1 activity comprising contacting said APOL1 with at least one entity according to any one of embodiments 247 to 260 or a pharmaceutical composition according to embodiment 261.
   268. The method according to embodiment 267, wherein the APOL1 is associated with APOL1 genetic alleles chosen from homozygous G: S342G:I384M and homozygous G2: N388del:Y389del.
   269. The method according to embodiment 267, wherein the APOL1 is associated with compound heterozygous G1: S342G:I384M and G2: N388del:Y389del APOL1 genetic alleles.
   270. Use of glutaric acid cocrystal Form A of Compound I according to any one of embodiments 247 to 260 in the manufacture of a medicament for treating APOL1 mediated kidney disease.
   271. Glutaric acid cocrystal Form A of Compound I according to any one of embodiments 247-260 or the pharmaceutical composition according to embodiment 261 for use in treating APOL1 mediated kidney disease.
   272. A method of preparing glutaric acid cocrystal Form A of Compound I comprising combining Compound I Form A and glutaric acid with butyl acetate/toluene; stirring magnetically at room temperature and adding butyl acetate/toluene to maintain a fluid slurry;
centrifuging after about one week and removing remaining fluid;
drying solid in vacuum dessicator for 2-3 hours to provide glutaric acid cocrystal Form A of Compound I
   273. L-proline cocrystal Form A cocrystal of Compound I.
   274. L-proline cocrystal Form A of Compound I according to embodiment 273, characterized by an X-ray powder diffractogram having a signal at one or more two-theta values selected from 22.9±0.2, 20.2±0.2, 6.0±0.2, and 4.9±0.2.
   275. L-proline cocrystal Form A of Compound I according to embodiment 273, characterized by an X-ray powder diffractogram having a signal at two or more two-theta values selected from 22.9±0.2, 20.2±0.2, 6.0±0.2, and 4.9±0.2.
   276. L-proline cocrystal Form A of Compound I according to embodiment 273, characterized by an X-ray powder diffractogram having a signal at three or more two-theta values selected from 22.9±0.2, 20.2±0.2, 6.0±0.2, and 4.9±0.2.
   277. L-proline cocrystal Form A of Compound I according to embodiment 273, characterized by an X-ray powder diffractogram having signals at 22.9±0.2, 20.2±0.2, 6.0±0.2, and 4.9±0.2.
   278. L-proline cocrystal Form A of Compound I according to embodiment 273, characterized by an X-ray powder diffractogram having (a) a signal at the following two-theta values 22.9±0.2, 20.2±0.2, 6.0±0.2, and 4.9±0.2 and (b) a signal at one or more two-theta values selected from 24.3±0.2, 22.0±0.2, 19.5±0.2, and 17.9±0.2.
   279. L-proline cocrystal Form A of Compound I according to embodiment 273, characterized by an X-ray powder diffractogram having (a) a signal at the following two-theta values 22.9±0.2, 20.2±0.2, 6.0±0.2, and 4.9±0.2; and (b) a signal at two or more two-theta values selected from 24.3±0.2, 22.0±0.2, 19.5±0.2, and 17.9±0.2.
   280. L-proline cocrystal Form A of Compound I according to embodiment 273, characterized by an X-ray powder diffractogram having (a) a signal at the following two-theta values 22.9±0.2, 20.2±0.2, 6.0±0.2, and 4.9±0.2; and (b) a signal at three or more two-theta values selected from 24.3±0.2, 22.0±0.2, 19.5±0.2, and 17.9±0.2.
   281. L-proline cocrystal Form A of Compound I according to embodiment 273, characterized by an X-ray powder diffractogram having signals at 24.3±0.2, 22.9±0.2, 22.0±0.2, 20.2±0.2, 19.5±0.2, 17.9±0.2, 6.0±0.2, and 4.9±0.2 two-theta.
   282. L-proline cocrystal Form A of Compound I according to embodiment 273, characterized by an X-ray powder diffractogram substantially similar to that in FIG. 36.
   283. L-proline cocrystal Form A of Compound I according to embodiment 273, characterized by a DSC substantially similar to that in FIG. 38.
   284. L-proline cocrystal Form A of Compound I according to embodiment 273, characterized by a DSC having three endotherms at about 140° C., about 221° C., and about 232° C.
   285. L-proline cocrystal Form A of Compound I according to embodiment 273, characterized a TGA substantially similar to that in FIG. 37.
   286. L-proline cocrystal Form A of Compound I according to embodiment 273, characterized a TGA showing weight loss of about 6% from ambient temperature to about 130° C.
   287. A pharmaceutical composition comprising L-proline cocrystal Form A of Compound I according to any one of embodiments 273 to 286 and a pharmaceutically acceptable carrier.
   288. A method of treating APOL1 mediated kidney disease comprising administering to a patient in need thereof. L-proline cocrystal Form A of Compound I according to any one of embodiments 273 to 286 or a pharmaceutical composition according to embodiment 287.
   289. The method according to embodiment 288, wherein the APOL1 mediated kidney disease is chosen from ESKD, NDKD, FSGS, HIV-associated nephropathy, arterionephrosclerosis, lupus nephritis, microalbuminuria, and chronic kidney disease.
   290. The method according to embodiment 288, wherein the APOL1 mediated kidney disease is chosen from ESKD, NDKD, and FSGS.
   291. The method according to any one of embodiments 288-290, wherein the APOL1 mediated kidney disease is associated with APOL1 genetic alleles chosen from homozygous G1: S342G:I384M and homozygous G2: N388del:Y389del.
   292. The method according to any one of embodiments 288-290, wherein the APOL1 mediated kidney disease is associated with compound heterozygous G1: S342G:I384M and G2: N388del:Y389del APOL1 genetic alleles.
   293. A method of inhibiting APOL1 activity comprising contacting said APOL1 with at least one entity according to any one of embodiments 273 to 286 or a pharmaceutical composition according to embodiment 261.
   294. The method according to embodiment 293, wherein the APOL1 is associated with APOL1 genetic alleles chosen from homozygous G: S342G:I384M and homozygous G2: N388del:Y389del.
   295. The method according to embodiment 293, wherein the APOL1 is associated with compound heterozygous G1: S342G:I384M and G2: N388del:Y389del APOL1 genetic alleles.
   296. Use of L-proline cocrystal Form A of Compound I according to any one of embodiments 273 to 286 in the manufacture of a medicament for treating APOL1 mediated kidney disease.
   297. L-proline cocrystal Form A of Compound I according to any one of embodiments 273-286 or the pharmaceutical composition according to embodiment 287 for use in treating APOL1 mediated kidney disease.
   298. A method of preparing L-proline cocrystal Form A of Compound I comprising
mixing Compound I Form A with L-proline in a ball mill with pentanol;
milling at 100 hertz for about 30 minutes;
isolating L-proline cocrystal Form A of Compound I.
   299. L-proline cocrystal Form B cocrystal of Compound I.
   300. L-proline cocrystal Form B of Compound I according to embodiment 299, characterized by an X-ray powder diffractogram having a signal at one or more two-theta values selected from 22.5±0.2, 21.2±0.2, and 18.7±0.2.
   301. L-proline cocrystal Form B of Compound I according to embodiment 299, characterized by an X-ray powder diffractogram having a signal at two or more two-theta values selected from 22.5±0.2, 21.2±0.2, and 18.7±0.2.
   302. L-proline cocrystal Form B of Compound I according to embodiment 299, characterized by an X-ray powder diffractogram having signals at 22.5±0.2, 21.2±0.2, and 18.7±0.2.
   303. L-proline cocrystal Form B of Compound I according to embodiment 299, characterized by an X-ray powder diffractogram having (a) a signal at the following two-theta values 22.5±0.2, 21.2±0.2, and 18.7±0.2 and (b) a signal at one or more two-theta values selected from 28.5±0.2, 16.0±0.2, and 13.1±0.2.
   304. L-proline cocrystal Form B of Compound I according to embodiment 299, characterized by an X-ray powder diffractogram having (a) a signal at the following two-theta values 22.5±0.2, 21.2±0.2, and 18.7±0.2; and (b) a signal at two or more two-theta values selected from 28.5±0.2, 16.0±0.2, and 13.1±0.2.
   305. L-proline cocrystal Form B of Compound I according to embodiment 299, characterized by an X-ray powder diffractogram having signals at 28.5±0.2, 22.5±0.2, 21.2±0.2, 18.7±0.2, 16.0±0.2, and 13.1±0.2 two-theta.
   306. L-proline cocrystal Form B of Compound I according to embodiment 299, characterized by an X-ray powder diffractogram substantially similar to that in FIG. 39A.
   306a. L-proline cocrystal Form B of Compound I, characterized by a ¹³C NMR spectrum having a signal at three or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306b. L-proline cocrystal Form B of Compound I according to any one of embodiments 299-306, characterized by a ¹³C NMR spectrum having a signal at five or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306c. L-proline cocrystal Form B of Compound I according to any one of embodiments 299-306, characterized by a ¹³C NMR spectrum having a signal at seven or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306d. L-proline cocrystal Form B of Compound I according to any one of embodiments 299-306, characterized by a ¹³C NMR spectrum having a signal at ten or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306e. L-proline cocrystal Form B of Compound I according to any one of embodiments 299-306, characterized by a ¹³C NMR spectrum having a signal at twelve or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306f. L-proline cocrystal Form B of Compound I according to any one of embodiments 299-306, characterized by a ¹³C NMR spectrum having a signal at fifteen or more ppm values chosen from 175.9±0.2 ppm, 173.6±0.2 ppm, 172.3±0.2 ppm, 136.5±0.2 ppm, 130.3±0.2 ppm, 128.0±0.2 ppm, 120.0±0.2 ppm, 118.7±0.2 ppm, 118.2±0.2 ppm, 116.0±0.2 ppm, 110.2±0.2 ppm, 47.4±0.2 ppm, 46.9±0.2 ppm, 34.2±0.2 ppm, 31.8±0.2 ppm, 27.6±0.2 ppm, 26.6±0.2 ppm, 25.3±0.2 ppm, and 19.3±0.2 ppm.
   306g. L-proline cocrystal Form B of Compound I, characterized by a ¹³C NMR spectrum substantially similar to that in FIG. 39B.
   306h. L-proline cocrystal Form B of Compound I, characterized by a ¹⁹F NMR spectrum having a signal at −116.9±0.2 ppm.
   306i. L-proline cocrystal Form B of Compound I, characterized by a ¹⁹F NMR spectrum substantially similar to that in FIG. 39C.
   307. L-proline cocrystal Form B of Compound I according to embodiment 299, characterized by a DSC substantially similar to that in FIG. 41.
   308. L-proline cocrystal Form B of Compound I according to embodiment 299, characterized by a DSC having two endotherms at about 220° C. and about 232° C.
   309. L-proline cocrystal Form B of Compound I according to embodiment 299, characterized a TGA substantially similar to that in FIG. 40.
   310. L-proline cocrystal Form B of Compound I according to embodiment 299, characterized a TGA showing weight loss of about 1.6% from ambient temperature to about 200° C.
   311. A pharmaceutical composition comprising L-proline cocrystal Form B of Compound I according to any one of embodiments 299 to 310 and a pharmaceutically acceptable carrier.
   312. A method of treating APOL1 mediated kidney disease comprising administering to a patient in need thereof. L-proline cocrystal Form B of Compound I according to any one of embodiments 299 to 310 or a pharmaceutical composition according to embodiment 311.
   313. The method according to embodiment 312, wherein the APOL1 mediated kidney disease is chosen from ESKD, NDKD, FSGS, HIV-associated nephropathy, arterionephrosclerosis, lupus nephritis, microalbuminuria, and chronic kidney disease.
   314. The method according to embodiment 312, wherein the APOL1 mediated kidney disease is chosen from ESKD, NDKD, and FSGS.
   315. The method according to any one of embodiments 312-314, wherein the APOL1 mediated kidney disease is associated with APOL1 genetic alleles chosen from homozygous G1: S342G:I384M and homozygous G2: N388del:Y389del.
   316. The method according to any one of embodiments 312-314, wherein the APOL1 mediated kidney disease is associated with compound heterozygous G1: S342G:I384M and G2: N388del:Y389del APOL1 genetic alleles.
   317. A method of inhibiting APOL1 activity comprising contacting said APOL1 with at least one entity according to any one of embodiments 299 to 310 or a pharmaceutical composition according to embodiment 311.
   318. The method according to embodiment 317, wherein the APOL1 is associated with APOL1 genetic alleles chosen from homozygous G: S342G:I384M and homozygous G2: N388del:Y389del.
   319. The method according to embodiment 317, wherein the APOL1 is associated with compound heterozygous G1: S342G:I384M and G2: N388del:Y389del APOL1 genetic alleles.
   320. Use of L-proline cocrystal Form B of Compound I according to any one of embodiments 299 to 310 in the manufacture of a medicament for treating APOL1 mediated kidney disease.
   321. L-proline cocrystal Form B of Compound I according to any one of embodiments 299 to 310 or the pharmaceutical composition according to embodiment 311 for use in treating APOL1 mediated kidney disease.
   322. A method of preparing L-proline cocrystal Form B of Compound I comprising
mixing Compound I Form A with L-proline in a ball mill with butyl acetate;
milling at 100 hertz for about 30 minutes;
isolating L-proline cocrystal Form B of Compound I.
   323. Vanillin cocrystal Form A cocrystal of Compound I.
   324. Vanillin cocrystal Form A of Compound I according to embodiment 323, characterized by an X-ray powder diffractogram having a signal at one or more two-theta values selected from 24.5±0.2, 21.9±0.2, 21.0±0.2, 15.6±0.2, and 9.6±0.2.
   325. Vanillin cocrystal Form A of Compound I according to embodiment 323, characterized by an X-ray powder diffractogram having a signal at two or more two-theta values selected from 24.5±0.2, 21.9±0.2, 21.0±0.2, 15.6±0.2, and 9.6±0.2.
   326. Vanillin cocrystal Form A of Compound I according to embodiment 323, characterized by an X-ray powder diffractogram having a signal at three or more two-theta values selected from 24.5±0.2, 21.9±0.2, 21.0±0.2, 15.6±0.2, and 9.6±0.2.
   327. Vanillin cocrystal Form A of Compound I according to embodiment 323, characterized by an X-ray powder diffractogram having signals at 24.5±0.2, 21.9±0.2, 21.0±0.2, 15.6±0.2, and 9.6±0.2 two-theta.
   328. Vanillin cocrystal Form A of Compound I according to embodiment 323, characterized by an X-ray powder diffractogram having (a) a signal at the following two-theta values 24.5±0.2, 21.9±0.2, 21.0±0.2, 15.6±0.2, and 9.6±0.2 and (b) a signal at one or more two-theta values selected from 27.4±0.2, 26.7±0.2, 26.2±0.2, 23.7±0.2, and 14.3±0.2.
   329. Vanillin cocrystal Form A of Compound I according to embodiment 323, characterized by an X-ray powder diffractogram having (a) a signal at the following two-theta values 24.5±0.2, 21.9±0.2, 21.0±0.2, 15.6±0.2, and 9.6±0.2; and (b) a signal at two or more two-theta values selected from 27.4±0.2, 26.7±0.2, 26.2±0.2, 23.7±0.2, and 14.3±0.2.
   330. Vanillin cocrystal Form A of Compound I according to embodiment 323, characterized by an X-ray powder diffractogram having (a) a signal at the following two-theta values 24.5±0.2, 21.9±0.2, 21.0±0.2, 15.6±0.2, and 9.6±0.2; and (b) a signal at three or more two-theta values selected from 27.4±0.2, 26.7±0.2, 26.2±0.2, 23.7±0.2, and 14.3±0.2.
   331. Vanillin cocrystal Form A of Compound I according to embodiment 323, characterized by an X-ray powder diffractogram having signals at 27.4±0.2, 26.7±0.2, 26.2±0.2, 24.5±0.2, 23.7±0.2, 21.9±0.2, 21.0±0.2, 15.6±0.2, 14.3±0.2, and 9.6±0.2 two-theta.
   332. Vanillin cocrystal Form A of Compound I according to embodiment 299, characterized by an X-ray powder diffractogram substantially similar to that in FIG. 42A.
   332a. Vanillin cocrystal Form A of Compound I characterized by a ¹³C NMR spectrum having a signal at three or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332b. Vanillin cocrystal Form A of Compound I according to any one of embodiments 323-332, characterized by a ¹³C NMR spectrum having a signal at five or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332c. Vanillin cocrystal Form A of Compound I according to any one of embodiments 323-332, characterized by a ¹³C NMR spectrum having a signal at seven or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332d. Vanillin cocrystal Form A of Compound I according to any one of embodiments 323-332, characterized by a ¹³C NMR spectrum having a signal at ten or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332e. Vanillin cocrystal Form A of Compound I according to any one of embodiments 323-332, characterized by a ¹³C NMR spectrum having a signal at twelve or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332f Vanillin cocrystal Form A of Compound I according to any one of embodiments 323-332, characterized by a ¹³C NMR spectrum having a signal at fifteen or more ppm values chosen from 191.4±0.2 ppm, 175.4±0.2 ppm, 171.9±0.2 ppm, 153.7±0.2 ppm, 147.4±0.2 ppm, 130.6±0.2 ppm, 129.4±0.2 ppm, 128.8±0.2 ppm, 127.8±0.2 ppm, 121.9±0.2 ppm, 120.5±0.2 ppm, 119.2±0.2 ppm, 116.1±0.2 ppm, 114.6±0.2 ppm, 113.0±0.2 ppm, 110.7±0.2 ppm, 107.8±0.2 ppm, 44.5±0.2 ppm, 35.5±0.2 ppm, and 18.2±0.2 ppm.
   332g. Vanillin cocrystal Form A of Compound I, characterized by a ¹³C NMR spectrum substantially similar to that is FIG. 42 B.
   332h. Vanillin cocrystal Form A of Compound I, characterized by a ¹⁹F NMR spectrum having a signal at −115.2±0.2 ppm.
   332i. Vanillin cocrystal Form A of Compound I, characterized by a ¹⁹F NMR spectrum substantially similar to that is FIG. 42 C.
   333. Vanillin cocrystal Form A of Compound I according to embodiment 323, characterized by a DSC substantially similar to that in FIG. 44.
   334. Vanillin cocrystal Form A of Compound I according to embodiment 323, characterized by a DSC having an endotherm at about 136° C.
   335. Vanillin cocrystal Form A of Compound I according to embodiment 323, characterized a TGA substantially similar to that in FIG. 43.
   336. Vanillin cocrystal Form A of Compound I according to embodiment 323, characterized a TGA showing weight loss of about 25% from ambient temperature to about 200° C.
   337. A pharmaceutical composition comprising vanillin cocrystal Form A of Compound I according to any one of embodiments 323 to 336 and a pharmaceutically acceptable carrier.
   338. A method of treating APOL1 mediated kidney disease comprising administering to a patient in need thereof vanillin cocrystal Form A of Compound I according to any one of embodiments 323 to 336 or a pharmaceutical composition according to embodiment 337.
   339. The method according to embodiment 338, wherein the APOL1 mediated kidney disease is chosen from ESKD, NDKD, FSGS, HIV-associated nephropathy, arterionephrosclerosis, lupus nephritis, microalbuminuria, and chronic kidney disease.
   340. The method according to embodiment 338, wherein the APOL1 mediated kidney disease is chosen from ESKD, NDKD, and FSGS.
   341. The method according to any one of embodiments 338-340, wherein the APOL1 mediated kidney disease is associated with APOL1 genetic alleles chosen from homozygous G1: S342G:I384M and homozygous G2: N388del:Y389del.
   342. The method according to any one of embodiments 338-340, wherein the APOL1 mediated kidney disease is associated with compound heterozygous G1: S342G:I384M and G2: N388del:Y389del APOL1 genetic alleles.
   343. A method of inhibiting APOL1 activity comprising contacting said APOL1 with at least one entity according to any one of embodiments 323 to 336 or a pharmaceutical composition according to embodiment 337.
   344. The method according to embodiment 343, wherein the APOL1 is associated with APOL1 genetic alleles chosen from homozygous G: S342G:I384M and homozygous G2: N388del:Y389del.
   345. The method according to embodiment 343, wherein the APOL1 is associated with compound heterozygous G1: S342G:I384M and G2: N388del:Y389del APOL1 genetic alleles.
   346. Use of vanillin cocrystal Form A of Compound I according to any one of embodiments 323 to 336 in the manufacture of a medicament for treating APOL1 mediated kidney disease.
   347. Vanillin cocrystal Form A of Compound I according to any one of embodiments 323 to 336 or the pharmaceutical composition according to embodiment 337 for use in treating APOL1 mediated kidney disease.
   348. A method of preparing vanillin cocrystal Form A of Compound I comprising
mixing Compound I Form A with vanillin in a ball mill with pentanol;
milling at 100 hertz for about 30 minutes;
isolating vanillin cocrystal Form A of Compound I.
   349. 2-pyridone cocrystal Form A cocrystal of Compound I.
   350. 2-pyridone cocrystal Form A of Compound I according to embodiment 349, characterized by an X-ray powder diffractogram having a signal at one or more two-theta values selected from 19.5±0.2, 18.9±0.2, 15.8±0.2, 13.2±0.2, and 7.2±0.2.
   351. 2-pyridone cocrystal Form A of Compound I according to embodiment 349, characterized by an X-ray powder diffractogram having a signal at two or more two-theta values selected from 19.5±0.2, 18.9±0.2, 15.8±0.2, 13.2±0.2, and 7.2±0.2.
   352. 2-pyridone cocrystal Form A of Compound I according to embodiment 349, characterized by an X-ray powder diffractogram having a signal at three or more two-theta values selected from 19.5±0.2, 18.9±0.2, 15.8±0.2, 13.2±0.2, and 7.2±0.2.
   353. 2-pyridone cocrystal Form A of Compound I according to embodiment 349, characterized by an X-ray powder diffractogram having a signal four or more two-theta values selected from 19.5±0.2, 18.9±0.2, 15.8±0.2, 13.2±0.2, and 7.2±0.2 two-theta.
   354. 2-pyridone cocrystal Form A of Compound I according to embodiment 349, characterized by an X-ray powder diffractogram having signals at the following two-theta values 19.5±0.2, 18.9±0.2, 15.8±0.2, 13.2±0.2, and 7.2±0.2
   355. 2-pyridone cocrystal Form A of Compound I according to embodiment 349, characterized by an X-ray powder diffractogram substantially similar to that in FIG. 45.
   356. 2-pyridone cocrystal Form A of Compound I according to embodiment 349, characterized by a ¹³C NMR spectrum having one or more signals selected from 165.3±0.2 ppm, 136.1±0.2 ppm, 129.7±0.2 ppm, and 119.8±0.2 ppm.
   357. 2-pyridone cocrystal Form A of Compound I according to embodiment 349, characterized by a ¹³C NMR spectrum having two or more signals selected from 165.3±0.2 ppm, 136.1±0.2 ppm, 129.7±0.2 ppm, and 119.8±0.2 ppm.
   358. 2-pyridone cocrystal Form A of Compound I according to embodiment 349, characterized by a ¹³C NMR spectrum having three or more signals selected from 165.3±0.2 ppm, 136.1±0.2 ppm, 129.7±0.2 ppm, and 119.8±0.2 ppm.
   359. 2-pyridone cocrystal Form A of Compound I according to embodiment 349, characterized by a ¹³C NMR spectrum signals at 165.3±0.2 ppm, 136.1±0.2 ppm, 129.7±0.2 ppm, and 119.8±0.2 ppm.
   360. 2-pyridone cocrystal Form A of Compound I according to embodiment 349, characterized by a ¹³C NMR spectrum having (a) signals at 165.3±0.2 ppm, 136.1±0.2 ppm, 129.7±0.2 ppm, and 119.8±0.2 ppm; and (b) one or more signals selected from
   142.3±0.2 ppm, 135.2±0.2 ppm, 107.8±0.2 ppm, and 36.6±0.2 ppm.
   361. 2-pyridone cocrystal Form A of Compound I according to embodiment 349, characterized by a ¹³C NMR spectrum having (a) signals at 165.3±0.2 ppm, 136.1±0.2 ppm, 129.7±0.2 ppm, and 119.8±0.2 ppm; and (b) two or more signals selected from
   142.3±0.2 ppm, 135.2±0.2 ppm, 107.8±0.2 ppm, and 36.6±0.2 ppm.
   362. 2-pyridone cocrystal Form A of Compound I according to embodiment 349, characterized by a ¹³C NMR spectrum having (a) signals at 165.3±0.2 ppm, 136.1±0.2 ppm, 129.7±0.2 ppm, and 119.8±0.2 ppm; and (b) three or more signals selected from
   142.3±0.2 ppm, 135.2±0.2 ppm, 107.8±0.2 ppm, and 36.6±0.2 ppm.
   363. 2-pyridone cocrystal Form A of Compound I according to embodiment 349, characterized by a ¹³C NMR spectrum having signals at 165.3±0.2 ppm, 142.3±0.2 ppm, 136.1±0.2 ppm, 135.2±0.2 ppm, 129.7±0.2 ppm, 119.8±0.2 ppm, 107.8±0.2 ppm, and 36.6±0.2 ppm.
   364. 2-pyridone cocrystal Form A of Compound I according to embodiment 349, characterized by a ¹³C NMR spectrum substantially similar to that in FIG. 46B.
   365. 2-pyridone cocrystal Form A of Compound I according to embodiment 349, characterized by a ¹⁹F NMR spectrum having a signal at −112.1±0.2 ppm.
   366. 2-pyridone cocrystal Form A of Compound I according to embodiment 349, characterized by a ¹⁹F NMR spectrum having a signal at −115.5±0.2 ppm.
   367. 2-pyridone cocrystal Form A of Compound I according to embodiment 349, characterized by a ¹⁹F NMR spectrum having signals at −112.1±0.2 ppm and −115.5±0.2 ppm.
   368. 2-pyridone cocrystal Form A of Compound I according to embodiment 349, characterized by a ¹⁹F NMR spectrum substantially similar to that in FIG. 47B.
   369. 2-pyridone cocrystal Form A of Compound I according to embodiment 349, characterized by a DSC substantially similar to that in FIG. 49.
   370. 2-pyridone cocrystal Form A of Compound I according to embodiment 349, characterized by a DSC having three endotherms at about 102° C., about 123° C., and about

### 216° C.

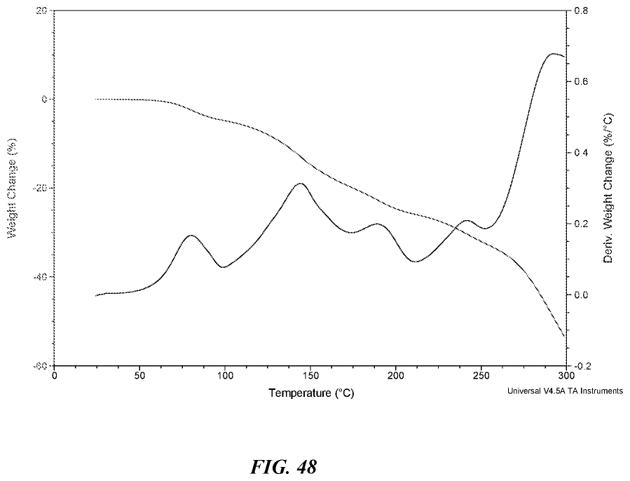

371. 2-pyridone cocrystal Form A of Compound I according to embodiment 349, characterized a TGA substantially similar to that in FIG. 48.
372. 2-pyridone cocrystal Form A of Compound I according to embodiment 349, characterized a TGA showing weight loss of about 25% from ambient temperature to about 200° C.
373. A pharmaceutical composition comprising 2-pyridone cocrystal Form A of Compound I according to any one of embodiments 349 to 372 and a pharmaceutically acceptable carrier.
374. A method of treating APOL1 mediated kidney disease comprising administering to a patient in need thereof 2-pyridone cocrystal Form A of Compound I according to any one of embodiments 349 to 372 or a pharmaceutical composition according to embodiment 373.
375. The method according to embodiment 374, wherein the APOL1 mediated kidney disease is chosen from ESKD, NDKD, FSGS, HIV-associated nephropathy, arterionephrosclerosis, lupus nephritis, microalbuminuria, and chronic kidney disease.
376. The method according to embodiment 374, wherein the APOL1 mediated kidney disease is chosen from ESKD, NDKD, and FSGS.
377. The method according to any one of embodiments 374-376, wherein the APOL1 mediated kidney disease is associated with APOL1 genetic alleles chosen from homozygous G1: S342G:I384M and homozygous G2: N388del:Y389del.
378. The method according to any one of embodiments 374-376, wherein the APOL1 mediated kidney disease is associated with compound heterozygous G1: S342G:I384M and G2: N388del:Y389del APOL1 genetic alleles.
379. A method of inhibiting APOL1 activity comprising contacting said APOL1 with at least one entity according to any one of embodiments 349 to 372 or a pharmaceutical composition according to embodiment 373.
380. The method according to embodiment 379, wherein the APOL1 is associated with APOL1 genetic alleles chosen from homozygous G: S342G:I384M and homozygous G2: N388del:Y389del.
381. The method according to embodiment 379, wherein the APOL1 is associated with compound heterozygous G1: S342G:I384M and G2: N388del:Y389del APOL1 genetic alleles.
382. Use of 2-pyridone cocrystal Form A of Compound I according to any one of embodiments 349 to 372 in the manufacture of a medicament for treating APOL1 mediated kidney disease.
383. 2-pyridone cocrystal Form A of Compound I according to any one of embodiments
349 to 372 or the pharmaceutical composition according to embodiment 373 for use in treating APOL1 mediated kidney disease.
384. A method of preparing 2-pyridone cocrystal Form A of Compound I comprising
mixing Compound I Form A with 2-pyridone in a ball mill with pentanol;
milling at 100 hertz for about 30 minutes;
isolating 2-pyridone cocrystal Form A of Compound I.

### EXAMPLES

In order that the disclosure described herein may be more fully understood, the following examples are set forth. It should be understood that these examples are for illustrative purposes only and are not to be construed as limiting this disclosure in any manner.

Methods of preparation, structure and physicochemical data of Compound I are reported in U.S. application Ser. No. 16/717,099 filed on Dec. 17, 2019, and PCT International Application No. PCT/US2019/066746 filed on Dec. 17, 2019, the contents of each of which are incorporated herein by reference.

### Example 1. Synthesis of Form a of Compound I

A. Preparation of Compound I and Forms Thereof

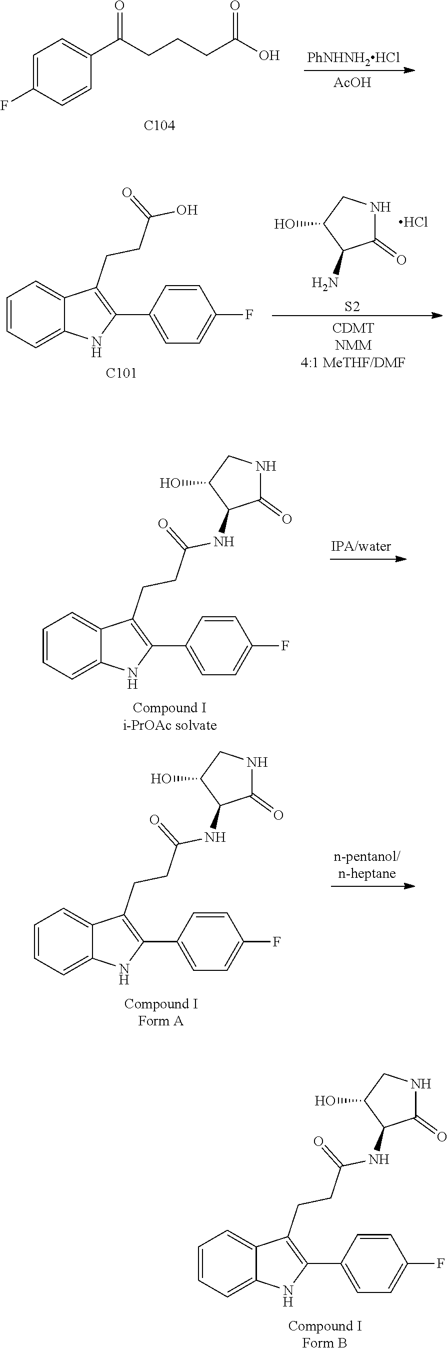

### Step 1. Synthesis of 3-[2-(4-fluorophenyl)-1H-indol-3-yl]propanoic acid (C101)

To a mixture of C104 (100.0 g, 1.0 equiv) and phenyl hydrazine hydrochloride (72.2 g, 1.05 eqiv) was charged AcOH (800 mL, 8 vol). The mixture was agitated and heated to 85° C. for 16 hours. The batch was cooled to 22° C. A vacuum was applied and the batch distill at <70° C. to ˜3 total volumes. The batch was cooled to 19-25° C. The reactor was charged with iPrOAc (800 mL, 8 vol) and then charged with water (800 mL, 8 vol). The internal temperature was adjusted to 20-25° C. and the biphasic mixture was stirred for no less than 0.5 h. Stirring was stopped and the phases allowed to separate for no less than 0.5 h. The lower aqueous layer was removed. 1 N HCl (500 mL, 5 vol) was charged to the reactor. The internal temperature was adjusted to 20-25° C., and the biphasic mixture was stirred for no less than 0.5 h. Stirring was stopped and phases were allowed to separate for no less than 0.5 h. The lower aqueous layer was removed. The reactor was charged with 1 N HCl (500 mL, 5 vol). The internal temperature was adjusted to 20-25° C., and the biphasic mixture was stirred for no less than 0.5 h. Stirring was stopped and phases were allowed to separate for no less than 0.5 h. The lower aqueous layer was removed. Water (500 mL, 5 vol) was charged to the reactor. The internal temperature was adjusted to 20-25° C., and the biphasic mixture was stirred for no less than 0.5 h. Stirring was stopped and phases were allowed to separate for no less than 0.5 h. The lower aqueous layer was removed. Water (500 mL, 5 vol) was charged to the reactor. The internal temperature was adjusted to 20-25° C., and the biphasic mixture was stirred for no less than 0.5 h. Stirring was stopped and phases were allowed to separate for no less than 0.5 h. The lower aqueous layer was removed. The organic phase was distilled under vacuum at <75° C. to 3 total volumes. The reactor was charged with toluene (1000 mL, 10 vol). The organic phase was distilled under vacuum at <75° C. to 5 total volumes. The reactor was charged with toluene (1000 mL, 10 vol). The organic phase was distilled under vacuum at <75° C. to 5 total volumes. The resulting slurry was heated to an internal temperature of 85° C. until complete dissolution of solids was achieved. The mixture was allowed to stir for 0.5 h at 85° C. and then cooled to an internal temperature of 19-25° C. over 5 h. The mixture was allowed to stir at 25° C. for no less than 2 h. The slurry was filtered. The filter cake was washed with toluene (1×2 vol (200 mL) and 1×1.5 vol (150 mL)). The solids were dried under vacuum with nitrogen bleed at 60° C. to afford product C101 (95.03 g, 70%).

### Step 2. Synthesis of Compound I

A mixture of 3-[2-(4-fluorophenyl)-1H-indol-3-yl]propanoic acid C101 (50 g, 1.0 equiv), S2 hydrochloride (28.3 g, 1.05 equiv), and CDMT (34.1 g, 1.1 equiv) was charged with 2-MeTHF (200 mL, 4 vol) and DMF (50 mL, 1 vol) and the mixture was agitated. The internal temperature adjusted to ≤13° C. The reactor was charged with NMM (64.5 g, 3.5 equiv) over 1 h, while maintaining internal temperature ≤20° C. The internal temperature was adjusted to 25° C. and the batch was stirred at that temperature for 14 h. The batch was cooled to 10° C. and charged with water (250 mL, 5 vol) while keeping the internal temperature <20° C. The batch was then warmed to 20-25° C. Stirring was stopped, and the phases allowed to separate for 10 min. The lower aqueous phase was removed. The aqueous layer was back extracted with 2-MeTHF (2×200 mL, 2×4 vol) at

20-25° C. The combined organic phases were washed with 1 N HCl (500 mL, 10 vol) at
20-25° C. by mixing for 10 min and settling for 10 min. The lower aqueous phase was removed. The organic phases were washed with 0.25 N HCl (2×250 mL, 2×5 vol) at 20-25° C. by mixing for 10 min and settling for 10 min for each wash. Lower aqueous phases were removed after each wash. The organic phase was washed with water (250 mL, 5 vol) at 20-25° C. by mixing for 10 min and settling for 10 min. The reactor was charged with
20 wt % Nuchar RGC® and stirred for 4 h. The reaction mixture was filtered through a pad of Celite®. The reactor and Celite® pad were rinsed with 2-MeTHF. The combined organics were distilled under vacuum at <50° C. to 5 total volumes. The reactor was charged with iPrOAc (500 mL, 10 vol). The organic phase was distilled under vacuum at <50° C. to 5 total volumes. The mixture was charged with additional iPrOAc (400 mL, 8 vol) and distillation under vacuum was repeated. The mixture was charged with additional iPrOAc (250 mL, 5 vol), heated to an internal temperature of 75° C. and stirred for 5 h. The slurry was cooled to 25° C., over 5 h and stirred for no less than 12 h. The slurry was filtered and the filter cake washed with iPrOAc (2×50 mL, 2×1 vol). The solids were dried under vacuum with nitrogen bleed at 55-60° C. to afford Compound I as an iPrOAc solvate (60.38 g including 9.9% w/w iPrOAc, 80.8% yield).

B. Recrystallization to Form a of Compound I

Compound I as an iPrOAc solvate (17.16 g after correction for iPrOAc content, 1.0 equiv) was charged to a reactor. A mixture of IPA (77 mL, 4.5 vol) and water (137 mL, 8 vol) were charged to the reactor. The slurry was heated to an internal temperature of

75° C. The batch was cooled to an internal temperature of 25° C. over 10 h and then stirred at 25° C. for at least 12 h. The slurry was filtered. The filter cake was washed with 36/64 IPA/water (2×52 mL, 2×3 vol). The solids were dried under vacuum with nitrogen bleed at 60° C. to afford Compound I as a neat, crystalline form (Form A, 15.35 g, 89%).

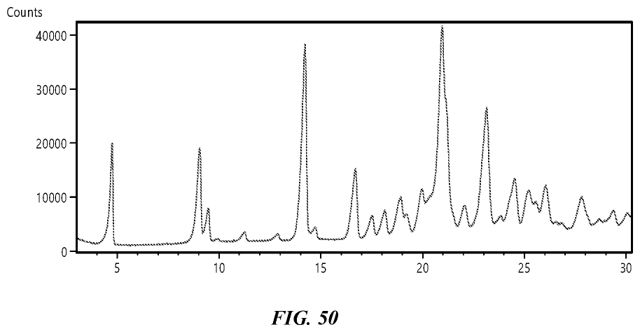

The X-ray powder diffractogram of Compound I Form A (FIG. 50) was acquired at room temperature using a PANalytical Empyrean diffractometer equipped with PIXcel 1D detector. The peaks are listed in Table A below.

TABLE A
XRPD of Form A of Compound I

| Angle | Intensity |
|---|---|
| (Degrees 2-Theta ± 0.2) | % |
| 21.0 | 100.0 |
| 14.2 | 95.9 |
| 23.1 | 59.5 |
| 21.2 | 54.2 |
| 4.7 | 49.4 |
| 9.0 | 46.2 |
| 16.7 | 33.5 |
| 22.9 | 31.2 |
| 24.5 | 24.2 |
| 20.0 | 21.2 |
| 26.1 | 20.1 |
| 26.0 | 18.4 |
| 25.2 | 18.2 |
| 18.9 | 17.6 |
| 9.5 | 16.5 |
| 27.8 | 15.0 |
| 24.3 | 13.6 |
| 25.6 | 12.7 |
| 18.1 | 11.9 |
| 22.1 | 11.8 |
| 17.5 | 9.8 |

C. Solid State NMR

Bruker-Biospin 400 MHz wide-bore spectrometer equipped with Bruker-Biospin 4 mm HFX probe was used. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

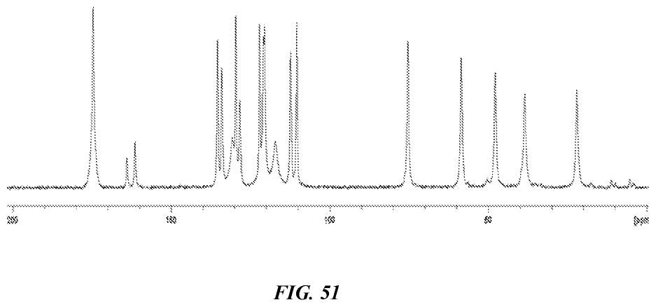

The ¹³C CPMAS of Form A (FIG. 51) was acquired at 275K with the following parameters: 12.5 kHz spinning; ref. adamantane 29.5 ppm. The peaks are listed in Table B below.

TABLE B
¹³C CPMAS of Compound I Form A

| Chem Shift [ppm ± 0.2] | Intensity [rel] |
|---|---|
| 174.5 | 100.0 |
| 163.8 | 16.5 |
| 161.3 | 25.5 |
| 135.3 | 81.6 |
| 133.9 | 66.4 |
| 130.6 | 28.0 |
| 129.5 | 96.0 |
| 128.3 | 48.2 |
| 122.0 | 90.6 |
| 120.8 | 83.3 |
| 120.5 | 89.9 |
| 117.0 | 25.6 |
| 112.2 | 75.4 |
| 110.3 | 91.5 |
| 75.3 | 80.8 |
| 58.4 | 72.4 |
| 47.7 | 63.6 |
| 38.4 | 52.1 |
| 22.0 | 54.3 |

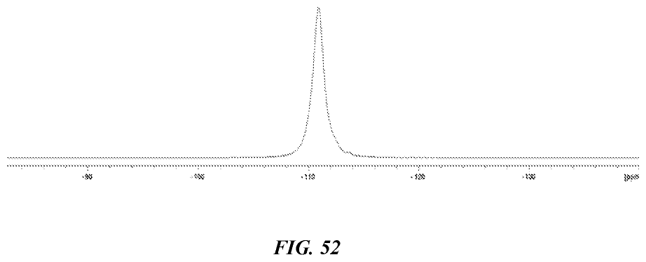

The ¹⁹F MAS of Form A (FIG. 52) was acquired at 275K with the following parameters: 12.5 kHz spinning; ref. adamantane 29.5 ppm. The peaks are listed in Table C below.

TABLE C
¹⁹F MAS of Compound I Form A

| Chem Shift [ppm ± 0.2] | Intensity [rel] |
|---|---|
| −110.9 | 12.5 |

### Example 2. Synthesis of Form B of Compound I

A. Recrystallization Form a of Compound I to Form B of Compound I

4.22 g of Form A of Compound I was charged with 33 mL 1-pentanol in a 100 mL reactor with overhead stirrer. The slurry was heated to 65° C. and held for 1 hour. Then the batch was seeded with 9.5 mg of Form B of Compound I and held at 65° C. for 11 hours. 50 mL heptane was charged over 24 hours. The slurry was cooled to 20° C. over 24 hours and held at 20° C. for 1 hour. The resulting solids were collected by vacuum filtration. The wet cake was transferred to a vacuum oven at 70° C. with a slight nitrogen bleed for 24 hours to yield 3.45 g of Form B of Compound I.

### Crystallization of Form B of Compound Ifrom Form a of Compound I

A jacketed reactor with an overhead stirrer, condenser, nitrogen line, temperature probe and recirculating fluid chiller/heater is charged with Compound I (Form A, 1.0 equiv). The reactor is charged with n-pentanol (8 vol). The mixture is heated to an internal temperature of 65° C. and forms thin slurry. The temperature is held at 65° C. for 12 hours. n-Heptane (18 vol, dry solvent) is added over 24 hours while maintaining an internal temperature of 65° C. The slurry becomes thick after 5-10 vols. of n-heptane is added. The stirring rate may need to be adjusted to maintain slurry mobility. The batch is cooled to an internal temperature of 20° C. over 12 hours. The slurry is filtered. The solids are dried under vacuum with nitrogen bleed at 70° C. to provide Compound I Form B.

B. X-Ray Powder Diffraction

The X-ray powder diffractogram of Form B of Compound I was acquired at room temperature using a PANalytical Empyrean diffractometer equipped with PIXcel 1D detector. Peaks were identified after background correction and refinement of peak profiles.

Interestingly, different lots of Compound I Form B show some variation in XRPD peaks but very little to no variability in solid state NMR peaks. The XRPD signals of Compound I Form B—Lot Tare listed in Table TA and shown in FIG. 1A. The XRPD signals of Compound I Form B—Lot 2 are listed in Table 1B and shown in FIG. 1B.

TABLE 1A
XRPD Peaks for Compound I Form B—Lot 1

| Angle | Intensity |
|---|---|
| (Degrees 2-Theta ± 0.2) | % |
| 14.2 | 100.0 |
| 23.3 | 47.0 |
| 4.7 | 22.8 |
| 21.1 | 14.7 |
| 20.3 | 14.2 |
| 9.2 | 11.1 |

TABLE 1B
XRPD peaks from Compound I Form B—Lot 2

| Angle | Intensity |
|---|---|
| (Degrees 2-Theta ± 0.2) | % |
| 14.3 | 100 |
| 23.4 | 64.8 |
| 21.2 | 36.3 |
| 4.7 | 22.3 |
| 20.4 | 21.0 |
| 16.9 | 13.4 |
| 9.3 | 12.2 |
| 9.6 | 5.4 |

A comparison of the XRPD diffractograms from Compound I Form B—Lot 1 and Lot 2 is shown in FIG. 1C. The top line represents Form B Lot 1 and the bottom line represents Form B Lot 2. As can be seen from FIG. 1C, in spite of the variability in XRPD peaks for Compound I Form B Lots 1 and 2, the overall XRPD pattern is very similar. FIG. 1D shows the substantial similarity of XRPD patterns for six separate preparations of Compound I Form B.

Compound 1 Form B is an intrinsically disordered material. Form B is the most thermodynamically stable form of Compound 1 from about 0 to 0.5 or 0.6 water activity at room temperature. Without being bound by theory, it is possible that the differences in XRPD peaks are the result of the level of residual solvent found in the lots. It should also be noted that the XRPD peaks listed for Compound I Form B overlap significantly with Compound I Form A. Unique peaks are listed in bold in Tables TA and 1B. This significant overlap is another reason why solid state NMR is a better way to distinguish between Compound I Form A and Compound I Form B.

C. Solid State NMR

The ¹³C CPMAS of Form B of Compound I (FIG. 2) was acquired at 275K with

12.5 kHz spinning and using as a reference adamantane 29.5 ppm. The peaks are listed in Table 2 below.

TABLE 2
Peak list from ¹³C CPMAS of Form B

| Chem Shift [ppm ± 0.2] | Intensity [rel] |
|---|---|
| 175.9 | 71.7 |
| 174.6 | 47.2 |
| 172.3 | 33.7 |
| 163.9 | 13.0 |
| 163.3 | 12.8 |
| 161.9 | 20.9 |
| 161.2 | 19.6 |
| 135.7 | 62.4 |
| 134.2 | 47.2 |
| 132.9 | 42.5 |
| 130.1 | 78.4 |
| 129.5 | 100.0 |
| 127.9 | 30.7 |
| 124.3 | 9.5 |
| 120.8 | 83.6 |
| 119.4 | 57.9 |
| 118.2 | 45.1 |
| 116.2 | 27.2 |
| 114.7 | 29.2 |
| 113.5 | 41.5 |
| 112.4 | 39.1 |
| 111.5 | 44.3 |
| 110.5 | 39.2 |
| 75.7 | 75.7 |
| 75.0 | 48.6 |
| 73.1 | 9.0 |
| 59.2 | 44.0 |
| 57.2 | 42.1 |
| 47.0 | 52.7 |
| 35.0 | 36.9 |
| 33.3 | 45.1 |
| 21.5 | 13.8 |
| 20.4 | 24.3 |
| 19.5 | 25.1 |
| 18.8 | 11.5 |
| 17.6 | 29.5 |
| 16.7 | 13.5 |

The ¹⁹F MAS of Form B of Compound I (FIG. 3) was acquired at 275K with

12.5 kHz spinning and using as a reference adamantane 29.5 ppm. The peaks are listed in Table 3 below.

TABLE 3
Peak list from ¹⁹F MAS of Form B

| Chem Shift [ppm ± 0.2] | Intensity [rel] |
|---|---|
| −109.4 | 5.8 |
| −112.5 | 12.5 |
| −113.7 | 3.8 |

As noted above, the various lots of Compound 1 Form B showed little to no variability in ssNMR patterns. It can also be seen that there is very little overlap in the ssNMR data for Compound I Form A and Form B. The peaks that are unique to Form B are shown in bold in Tables 2 and 3 above.

D. Thermogravimetric Analysis

Thermal gravimetric analysis of Form B of Compound I was measured using a TA Instruments Q5000 TGA. The TGA thermogram (FIG. 4) shows weight loss of ˜0.3% w/w from ambient temperature up to 225° C.

E. Differential Scanning Calorimetry Analysis

The melting point of Form B of Compound I was measured using a TA Instruments Discovery DSC. The thermogram (FIG. 5) shows a melting onset of 168° C. with a peak at 170° C. The melting peak of this form could range from 167° C. to 171° C.

F. Infrared (IR) Spectrum

The IR spectrum of Form B of Compound I was collected using Thermo Scientific Nicolet iS50 Spectrometer equipped with a diamond ATR sampling accessory. The IR spectra is provided at FIG. 6 and the peaks are listed in Table 4 below.

TABLE 4
Peak list from IR Spectrum of Form B

| Frequency [cm⁻¹] | Moiety |
|---|---|
| 3380 | Indole N—H |
| 3229 | O—H |
| 1716, 1695 | Lactam C═O |
| 1652 | Amide C═O |
| 1538, 1507, 1458 | Aromatic and |
| | heteroaromatic ring |
| 1227 | C—F |

### Example 3: Cocrystal Forms of Compound I

Solid State NMR experimental—Applies to all cocrystalforms of Compound I

A Bruker-Biospin 400 MHz wide-bore spectrometer equipped with Bruker-Biospin 4 mm HFX probe was used to evaluate cocrystal samples. Samples were packed into 4 mm ZrO₂ rotors and spun under Magic Angle Spinning (MAS) condition with spinning speed typically set to 12.5 kHz. The proton relaxation time was measured using ¹H MAS T₁ saturation recovery relaxation experiment in order to set up proper recycle delay of the ¹³C cross-polarization (CP) MAS experiment. The fluorine relaxation time was measured using ¹⁹F MAS T₁ saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### 1. Compound I Citric Acid Cocrystal Form A

A. Synthetic Procedure

Approximately 258 mg of Compound I form A and ˜126 mg of citric acid was added in a 4 mL vial. The mixture was dissolved in 3 ml of 2-Butanone (MEK). A slurry was formed after stirring with magnetic stirrer for about 30 min-1 hr. The slurry was centrifuged and the solid was dried in vacuum oven at 55° C. overnight with nitrogen bleed. Compound I citric acid cocrystal Form A was isolated.

In an alternative procedure, 15.70 g of citric acid was charge with 450 mL 2-butanone in a 500 mL bottle with magnetic stir bar. The slurry was heated to 50° C. and held for 30 min until solids were fully dissolved. 30.00 g of Compound I Form A was charged with 450 mL 2-butanone in a 1000 mL reactor with overhead stirrer. The slurry was heated to 40° C. and solids were fully dissolved. 225 mL of the prepared citric acid solution was charged into the reactor over 1.5 hours. Then the batch was seeded with 25 mg of Compound I citric acid cocrystal and held at 40° C. for 1 hour. The rest of the prepared citric acid solution was charged over 6 hours. The slurry was cooled to 25° C. over 3 hours and held at 25° C. before isolation. The resulting solids were collected by vacuum filtration. The wet cake was transferred to a vacuum oven at 45° C. with a slight nitrogen bleed for 24 hours to yield 32.84 g of product, Compound I citric acid cocrystal Form A.

B. X-Ray Powder Diffraction:

The XRPD diffractogram of Compound I citric acid cocrystal Form A (FIG. 7) was acquired at room temperature in reflection mode using a Bruker Advance equipped with Vantec-1 detector. The sample was analyzed on a silicon sample holder from 3-40° 2-theta on continuous mode with step size of 0.0144531° and time per step of 0.25 seconds. Sample was spinning at 15 rpm.

TABLE 5
XRPD diffractogram of Compound I citric acid cocrystal Form A

| | Angle (Degrees 2- | |
|---|---|---|
| XRPD Peaks | Theta ± 0.2) | Intensity % |
| 1 | 19.5 | 100.0 |
| 2 | 24.4 | 77.3 |
| 3 | 14.6 | 73.1 |
| 4 | 4.9 | 71.6 |
| 5 | 22.2 | 28.9 |
| 6 | 18.2 | 11.8 |
| 7 | 9.2 | 11.2 |
| 8 | 18.3 | 10.7 |
| 9 | 21.2 | 10.4 |

C. Solid State NMR

The ¹³C CPMAS of the citric acid cocrystal Form A of Compound I (FIG. 8) was acquired at 275K with 12.5 kHz spinning and using adamantane as a reference. The peaks are listed in Table 6 below.

TABLE 6
Peak list from ¹³C CPMAS of Compound I citric acid
cocrystal Form A

| Peak # | Chem Shift [ppm ± 0.2] | Intensity [rel] |
|---|---|---|
| 1 | 179.9 | 73.2 |
| 2 | 178.6 | 53.2 |
| 3 | 177.0 | 36.9 |
| 4 | 174.8 | 79.5 |
| 5 | 173.8 | 82.7 |
| 6 | 163.9 | 6.6 |
| 7 | 161.5 | 7.9 |
| 8 | 135.8 | 40.5 |
| 9 | 133.6 | 24.9 |
| 10 | 130.1 | 87.9 |
| 11 | 129.4 | 65.7 |
| 12 | 125.2 | 7.1 |
| 13 | 122.4 | 72.0 |
| 14 | 120.1 | 55.0 |
| 15 | 116.3 | 77.2 |
| 16 | 112.0 | 59.1 |
| 17 | 111.2 | 42.4 |
| 18 | 74.8 | 100.0 |
| 19 | 71.8 | 78.1 |
| 20 | 56.5 | 41.5 |
| 21 | 54.5 | 5.1 |
| 22 | 49.6 | 42.5 |
| 23 | 46.9 | 36.6 |
| 24 | 44.1 | 62.0 |
| 25 | 37.7 | 43.6 |
| 26 | 36.0 | 9.2 |
| 27 | 22.8 | 41.1 |
| 28 | 21.2 | 7.5 |

The ¹⁹F MAS of citric acid cocrystal Form A of Compound I (FIG. 9) was acquired at 275K with 12.5 kHz spinning and using adamantane as a reference. The peaks are listed in Table 7 below.

TABLE 7
Peak list from ¹⁹F MAS of citric acid cocrystal Form A of Compound I

| | Chem Shift [ppm ± | |
|---|---|---|
| Peak # | 0.2] | Intensity [rel] |
| 1 | −112.6 | 0.6 |
| 2 | −114.8 | 1.8 |
| 3 | −116.8 | 12.5 |

D. Thermogravimetric Analysis:

Thermal gravimetric analysis of Compound I Citric acid cocrystal Form A was measured using the TA Instruments Q5000 TGA. The TGA thermogram (FIG. 10) shows negligible weight loss from ambient temperature up until thermal degradation.

E. Differential Scanning Calorimetry Analysis:

The melting point of Compound I Citric acid cocrystal Form A was measured using a TA Instruments Q2000 DSC. The thermogram (FIG. 11) shows an endotherm at

### 189° C.

### 2. Compound I Piperazine Cocrystal Form A

A. Synthetic Procedure:

Compound I Form A ˜50 mg and ˜11 mg of piperazine was weighed and 0.5 mL of ethyl acetate (pre saturated with piperazine) was added in 2 mL Eppendorf tube. The tube was placed in a sonication bath and sonicated for 30 minutes at ambient temperature. The solid isolated from this procedure is Compound I piperazine cocrystal Form A.

B. X-Ray Powder Diffraction:

The XRPD diffractogram of Compound I piperazine cocrystral Form A (FIG. 12) was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40°2θ with a step size of 0.0131303° and 49 s per step.

TABLE 8
XRPD diffractogram of Compound I piperazine cocrystal Form A

| | Angle (Degrees 2- | |
|---|---|---|
| XRPD Peaks | Theta ± 0.2) | Intensity % |
| 1 | 19.7 | 100.0 |
| 2 | 10.0 | 60.4 |
| 3 | 17.3 | 56.0 |
| 4 | 13.1 | 52.5 |
| 5 | 16.9 | 46.7 |
| 6 | 22.2 | 41.0 |
| 7 | 22.0 | 39.2 |
| 8 | 26.5 | 35.1 |
| 9 | 16.3 | 29.6 |
| 10 | 13.4 | 28.6 |
| 11 | 24.8 | 28.4 |
| 12 | 16.7 | 25.8 |
| 13 | 22.7 | 25.3 |
| 14 | 26.2 | 23.7 |
| 15 | 8.8 | 23.0 |
| 16 | 19.3 | 20.3 |
| 17 | 27.9 | 18.6 |
| 18 | 23.6 | 17.8 |
| 19 | 15.1 | 17.1 |
| 20 | 21.4 | 16.4 |
| 21 | 21.1 | 15.0 |
| 22 | 23.8 | 14.0 |
| 23 | 17.6 | 12.0 |

C. Solid State NMR

The ¹³C CPMAS of the piperazine cocrystal Form A of Compound I (FIG. 13) was acquired at 275K with 12.5 kHz spinning and using adamantane as a reference. The peaks are listed in Table 9 below.

TABLE 9
Peak list from ¹³C CPMAS of Compound I piperazine cocrystal Form A

| | Chem Shift [ppm ± | |
|---|---|---|
| Peak # | 0.2] | Intensity [rel] |
| 1 | 177.6 | 24.8 |
| 2 | 177.3 | 31.0 |
| 3 | 173.4 | 22.0 |
| 4 | 173.0 | 36.1 |
| 5 | 163.3 | 7.9 |
| 6 | 160.6 | 13.2 |
| 7 | 136.2 | 54.3 |
| 8 | 130.5 | 60.1 |
| 9 | 129.2 | 59.3 |
| 10 | 129.0 | 61.7 |
| 11 | 125.2 | 52.4 |
| 12 | 120.5 | 59.3 |
| 13 | 119.9 | 64.1 |
| 14 | 113.3 | 41.6 |
| 15 | 111.6 | 64.4 |
| 16 | 111.0 | 65.2 |
| 17 | 72.8 | 71.6 |
| 18 | 65.6 | 23.3 |
| 19 | 65.2 | 38.2 |
| 20 | 52.0 | 40.1 |
| 21 | 47.0 | 88.0 |
| 22 | 46.2 | 69.3 |
| 23 | 45.1 | 82.8 |
| 24 | 44.8 | 100.0 |
| 25 | 37.1 | 55.1 |
| 26 | 24.5 | 58.1 |

The ¹⁹F MAS of pierazine cocrystal Form A of Compound I (FIG. 14) was acquired at 275K with 12.5 kHz spinning and using adamantane as a reference. The peaks are listed in Table 10.

TABLE 10
Peak list from
¹⁹F MAS of piperazine cocrystal Form A of Compound I

| | Chem Shift | |
|---|---|---|
| Peak # | [ppm ± 0.2] | Intensity [rel] |
| 1 | −112.1 | 12.5 |

D. Thermogravimetric Analysis:

Thermal gravimetric analysis of Compound I piperazine cocrystal form A was measured using the TA Instruments Discovery TGA. The thermogram (FIG. 15) shows ˜15% weight loss from ambient to 115° C. with continued weight loss until 300° C.

E. Differential Scanning Calorimetry Analysis:

The melting point of Compound I Piperazine cocrystal Form A was measured using the TA Instruments Q2000 DSC. The sample was placed in an aluminum pan, then along with an empty aluminum reference pan in a calorimeter cell. The calorimeter cell was closed and scanned from 50° C. to 300° C. with modulation of 0.32° C. every 60 seconds and a heating rate of 2° C. per minute under a nitrogen flow. The thermogram (FIG. 16) shows multiple endothermic peaks around 123 and 130° C.

### 3. Compound I Urea Cocrystal Form A

A. Synthetic Procedure:

82 mg of Compound I Form A was dissolved in 3 ml of solvent (2-propanol) and then 8 mg of urea was dissolved in the same vial. The solution was stirred at ambient temperature for 1 hour. 200 mg of Compound I Form A and 63 mg of urea dry were manually ground for 5 minutes. In a separate vial 30-40 mg of the ground physical mixture was added to 750 ml of pre-saturated solution at ambient temperature made a slurry. The slurry was then heated to 25° C. and stirred for 24 hours. The solids were analyzed as Compound I Urea cocrystal Form A.

Alternatively Compound I urea cocrystal Form A is prepared by charging 5.00 g of Compound I Form A with 50 mL of solvent mixture (95 v % 2-butanone with 5 v % water) in a 100 mL reactor with overhead stirrer. The slurry was heated to 40° C. and solids were fully dissolved. 0.788 g of urea solids was charged into the reactor. The solids were fully dissolved. Then 2.5 g of Compound I Form A and 0.394 g of urea solids was charged into the reactor. The solids slowly dissolved. Then the batch was seeded with 5 mg of Compound I urea cocrystal and hold at 40° C. for 2.5 hours. The slurry was cool to

25° C. over 3 hours and hold at 25° C. before isolation. The resulting solids were collected by vacuum filtration. The wet cake was transferred to a vacuum oven at 45° C. with a slight nitrogen bleed for 72 hours to yield 1.13 g of product, Compound I urea cocrystal Form A.

B. X-Ray Powder Diffraction:

The XRPD diffractogram of Compound I urea cocrystral Form A (FIG. 17) was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40°2θ with a step size of 0.0131303° and 49 s per step.

TABLE 11
XRPD diffractogram of Compound I urea cocrystal Form A

| | Angle (Degrees 2- | |
|---|---|---|
| XRPD Peaks | Theta ± 0.2) | Intensity % |
| 1 | 22.4 | 100.0 |
| 2 | 20.4 | 88.1 |
| 3 | 18.4 | 66.0 |
| 4 | 20.3 | 54.1 |
| 5 | 20.6 | 49.9 |
| 6 | 21.2 | 43.8 |
| 7 | 9.4 | 39.8 |
| 8 | 21.4 | 38.1 |
| 9 | 21.1 | 32.6 |
| 10 | 21.7 | 28.1 |
| 11 | 23.3 | 25.0 |
| 12 | 18.1 | 23.1 |
| 13 | 3.8 | 21.4 |
| 14 | 25.5 | 20.8 |
| 15 | 29.3 | 16.0 |
| 16 | 15.2 | 15.8 |
| 17 | 19.5 | 13.9 |
| 18 | 26.6 | 13.3 |
| 19 | 14.7 | 10.3 |

C. Solid State NMR

The ¹³C CPMAS of the urea cocrystal Form A of Compound I (FIG. 18) was acquired at 275K with 12.5 kHz spinning and using adamantane as a reference. The peaks are listed in Table 12.

TABLE 12
¹³C CPMAS of Compound I urea cocrystal Form A

| Peak # | Chem Shift [ppm ± 0.2] | Intensity [rel] |
|---|---|---|
| 1 | 175.4 | 47.0 |
| 2 | 175.0 | 50.3 |
| 3 | 163.6 | 15.8 |
| 4 | 162.7 | 28.5 |
| 5 | 161.2 | 19.7 |
| 6 | 135.5 | 54.8 |
| 7 | 133.7 | 14.7 |
| 8 | 132.0 | 14.1 |
| 9 | 129.2 | 100.0 |
| 10 | 127.8 | 20.7 |
| 11 | 121.3 | 33.0 |
| 12 | 120.3 | 59.5 |
| 13 | 119.1 | 29.4 |
| 14 | 116.7 | 22.5 |
| 15 | 115.3 | 35.5 |
| 16 | 113.5 | 21.6 |
| 17 | 112.3 | 23.5 |
| 18 | 110.2 | 39.1 |
| 19 | 74.6 | 72.8 |
| 20 | 58.4 | 53.4 |
| 21 | 44.6 | 60.9 |
| 22 | 38.4 | 50.2 |
| 23 | 19.2 | 35.4 |
| 24 | 18.9 | 39.7 |

The ¹⁹F MAS of urea cocrystal Form A of Compound I (FIG. 19) was acquired at 275K with 12.5 kHz spinning and using adamantane as a reference. The peaks are listed in Table 10.

TABLE 13
Peak list from ¹⁹F MAS of urea cocrystal Form A of Compound I

| | Chem Shift | |
|---|---|---|
| Peak # | [ppm ± 0.2] | Intensity [rel] |
| 1 | −110.8 | 0.8 |
| 2 | −113.2 | 9.8 |
| 3 | −113.7 | 12.5 |

D. Thermogravimetric Analysis:

Thermal gravimetric analysis of Compound I urea cocrystal Form A was measured using the TA TGA Q5000 from TA Instruments. The sample was scanned from 25° C. to 250° C. with a heating rate of 10° C. per minute under a nitrogen purge. The TGA thermogram (FIG. 20) shows gradual weight loss of around 0.3% from ambient temperature until thermal degradation.

E. Differential Scanning Calorimetry Analysis:

Differential scanning calorimetry analysis of Compound I urea cocrystal Form A was measured using the Discovery DSC for TA Instruments. The sample was placed in an aluminum pan, then along with an empty aluminum reference pan in a calorimeter cell. The calorimeter cell was closed and scanned from 35° C. to 250° C. with modulation of 0.32° C. every 60 seconds and a heating rate of 2° C. per minute under a nitrogen flow. The thermogram (FIG. 21) showed an endothermic peak around 182° C.

### 4. Compound I Nicotinamide Cocrystal Form A

A. Synthetic Procedure:

48 mg of Compound I form A was dissolved in 3 ml of solvent (ethyl acetate) then 21 mg of nicotinamide was dissolved in the same vial. The solution was stirred at ambient temperature for 1 hour. 150 mg of Compound I Form A and 48 mg of nicotinamide dry was manually ground for 5 minutes. In a separate vial 30-40 mg of the ground physical mixture was added to 750 ml of pre-saturated solution at ambient temperature made a slurry. The slurry was then heated to 25° C. and stirred for 24 hours. The solids were analyzed to be Compound I nicotinamide cocrystal Form A.

B. X-Ray Powder Diffraction:

The XRPD diffractogram of Compound I nicotinamide cocrystral Form A (FIG. 22) was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40°2θ with a step size of 0.0131303° and 49 s per step.

TABLE 14
XRPD diffractogram of Compound I nicotinamide cocrystal Form A

| | | Intensity |
|---|---|---|
| XRPD Peaks | Angle (Degrees 2-Theta ± 0.2) | % |
| 1 | 5.1 | 100.0 |
| 2 | 15.3 | 13.7 |
| 3 | 6.3 | 7.9 |
| 4 | 19.6 | 5.5 |
| 5 | 18.3 | 5.3 |

C. Solid State NMR

The ¹³C CPMAS of the nicotinamide cocrystal Form A of Compound I (FIG. 23) was acquired at 275K with 12.5 kHz spinning and using adamantane as a reference. The peaks are listed in Table 12.

TABLE 15
¹³C CPMAS of Compound I nicotinamide cocrystal Form A

| | Chem Shift [ppm ± | |
|---|---|---|
| Peak # | 0.2] | Intensity [rel] |
| 1 | 174.5 | 57.3 |
| 2 | 173.4 | 41.1 |
| 3 | 171.7 | 8.4 |
| 4 | 167.4 | 41.8 |
| 5 | 164.1 | 8.8 |
| 6 | 162.9 | 8.5 |
| 7 | 161.7 | 13.8 |
| 8 | 160.4 | 10.6 |
| 9 | 153.3 | 34.1 |
| 10 | 152.1 | 30.3 |
| 11 | 149.2 | 67.2 |
| 12 | 136.1 | 75.9 |
| 13 | 135.5 | 47.7 |
| 14 | 134.0 | 8.2 |
| 15 | 131.2 | 15.8 |
| 16 | 130.6 | 16.7 |
| 17 | 129.0 | 61.9 |
| 18 | 128.3 | 100.0 |
| 19 | 123.8 | 32.1 |
| 20 | 122.4 | 28.7 |
| 21 | 121.2 | 66.5 |
| 22 | 120.4 | 31.9 |
| 23 | 119.2 | 62.1 |
| 24 | 118.2 | 40.7 |
| 25 | 115.7 | 13.3 |
| 26 | 115.0 | 13.3 |
| 27 | 112.7 | 65.4 |
| 28 | 112.0 | 67.2 |
| 29 | 71.4 | 75.1 |
| 30 | 60.5 | 47.8 |
| 31 | 59.6 | 23.7 |
| 32 | 46.8 | 27.3 |
| 33 | 44.5 | 26.9 |
| 34 | 37.4 | 45.9 |
| 35 | 36.9 | 51.6 |
| 36 | 21.3 | 13.3 |
| 37 | 18.3 | 33.5 |
| 38 | 17.5 | 42.7 |

The ¹⁹F MAS of nicotinamide cocrystal Form A of Compound I (FIG. 24) was acquired at 275K with 12.5 kHz spinning and using adamantane as a reference. The peaks are listed in Table 10.

TABLE 16
¹⁹F MAS of nicotinamide cocrystal Form A of Compound I

| | Chem Shift [ppm ± | |
|---|---|---|
| Peak # | 0.2] | Intensity [rel] |
| 1 | −116.4 | 11.5 |
| 2 | −117.9 | 12.5 |
| 3 | −118.5 | 11.6 |

D. Thermogravimetric Analysis:

Thermal gravimetric analysis of Compound I nicotinamide cocrystal Form A was measured using the TA TGA Q5000 from TA Instruments. The sample was scanned from 25° C. to 250° C. with a heating rate of 10° C. per minute under a nitrogen purge. The TGA thermogram (FIG. 25) shows weight loss of 7% from ambient temperature to 125° C.

E. Differential Scanning Calorimetry Analysis:

Differential scanning calorimetry analysis of Compound I nicotinamide cocrystal Form A was measured using the TA Q2000 DSC for TA Instruments. The sample was placed in an aluminum pan, then along with an empty aluminum reference pan in a calorimeter cell. The calorimeter cell was closed and scanned from 35° C. to 250° C. with modulation of 0.32° C. every 60 seconds and a heating rate of 2° C. per minute under a nitrogen flow. The thermogram (FIG. 26) showed an endothermic peak around 89° C.

### 5. Compound I Nicotinamide Cocrystal Form B

A. Synthetic Procedure:

100 mg of 1:1 stoichiometric equivalent mixture of Compound I Form A and Nicotinamide was placed in a steel ball mill vessel with 20 ul of pentanol. The ball mill was shaken at 15 Hertz for 30 minutes. The solids analyzed was Compound I nicotinamide cocrystal Form B.

B. X-Ray Powder Diffraction:

The XRPD diffractogram of Compound I Nicotinamide cocrystal Form B (FIG. 27) was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40°2θ with a step size of 0.0131303° and 49 s per step.

TABLE 17
XRPD diffractogram of Compound I nicitinomide cocrystal Form B

| | Angle (Degrees | |
|---|---|---|
| XRPD Peaks | 2-Theta ± 0.2) | Intensity % |
| 1 | 5.0 | 100.0 |
| 2 | 4.9 | 95.2 |
| 3 | 20.0 | 16.6 |
| 4 | 15.1 | 15.8 |
| 5 | 19.2 | 13.3 |
| 6 | 18.0 | 12.1 |
| 7 | 16.5 | 10.8 |
| 8 | 6.6 | 10.6 |

C. Solid State NMR

The ¹³C CPMAS of the nicotinamide cocrystal Form B of Compound I (FIG. 28) was acquired at 275K with 120.0 kHz spinning and using adamantane as a reference. The peaks are listed in Table 18.

TABLE 18
¹³C CPMAS of Compound I nicotinamide cocrystal Form B

| | Chem Shift [ppm | |
|---|---|---|
| Peak # | ± 0.2] | Intensity [rel] |
| 1 | 176.4 | 41.4 |
| 2 | 174.5 | 49.7 |
| 3 | 167.6 | 42.2 |
| 4 | 163.9 | 10.5 |
| 5 | 163.3 | 11.9 |
| 6 | 161.4 | 15.8 |
| 7 | 160.8 | 17.6 |
| 8 | 152.6 | 46.6 |
| 9 | 151.5 | 18.4 |
| 10 | 148.6 | 44.7 |
| 11 | 136.4 | 100.0 |
| 12 | 134.1 | 16.7 |
| 13 | 130.4 | 43.8 |
| 14 | 128.9 | 99.0 |
| 15 | 123.5 | 33.9 |
| 16 | 121.7 | 59.5 |
| 17 | 120.6 | 49.8 |
| 18 | 120.2 | 49.8 |
| 19 | 119.2 | 56.9 |
| 20 | 116.1 | 32.0 |
| 21 | 113.6 | 27.9 |
| 22 | 111.6 | 58.2 |
| 23 | 71.5 | 34.6 |
| 24 | 62.8 | 55.1 |
| 25 | 61.4 | 37.3 |
| 26 | 47.7 | 11.1 |
| 27 | 45.1 | 38.8 |
| 28 | 37.6 | 27.4 |
| 29 | 36.9 | 40.1 |
| 30 | 33.4 | 37.1 |
| 31 | 29.0 | 45.9 |
| 32 | 24.1 | 48.3 |
| 33 | 18.1 | 57.1 |
| 34 | 15.4 | 45.0 |

The ¹⁹F MAS of nicotinamide cocrystal Form Bof Compound I (FIG. 29) was PP4′ acquired at 275K with 120.0 kHz spinning and using adamantane as areference. The peaks are listed in Table 19.

TABLE 19
¹⁹F MAS of nicotinamide cocrystal Form B of Compound I

| Peak # | Chem Shift [ppm ± 0.2] | Intensity [rel] |
|---|---|---|
| 1 | −111.0 | 4.6 |
| 2 | −113.0 | 12.5 |
| 3 | −115.4 | 10.0 |

### 6. Compound I Aspartame Cocrystal Form A

A. Synthetic Procedure:

Approximately ˜30.1 mg of Compound I form A and ˜23.7 mg of aspartame was weighed and placed in ball mill with ˜10 ul of 1-Pentanol. The material was milled at 100 Hz for 30 minutes.

B. X-Ray Powder Diffraction:

The XRPD diffractogram of Compound I aspartame cocrystal Form A (FIG. 30) was acquired at room temperature using Rigaku Smart-Lab X-ray diffraction system. This system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

TABLE 20
XRPD diffractogram of Compound I aspartame cocrystal Form A

| XRPD Peaks | Angle (Degrees 2-Theta ± 0.2) | Intensity % |
|---|---|---|
| 1 | 6.9 | 100.0 |
| 2 | 20.6 | 89.2 |
| 3 | 21.2 | 68.5 |
| 4 | 20.3 | 54.6 |
| 5 | 22.7 | 53.4 |
| 6 | 18.5 | 47.1 |
| 7 | 16.0 | 30.0 |
| 8 | 7.4 | 29.4 |
| 9 | 21.6 | 28.7 |
| 10 | 24.0 | 28.1 |
| 11 | 26.4 | 27.7 |
| 12 | 9.3 | 26.6 |
| 13 | 22.0 | 24.4 |
| 14 | 13.7 | 24.2 |
| 15 | 11.6 | 23.4 |
| 16 | 16.1 | 20.7 |
| 17 | 18.9 | 20.3 |
| 18 | 15.7 | 19.8 |
| 19 | 12.2 | 18.1 |
| 20 | 12.5 | 18.0 |
| 21 | 28.3 | 18.0 |
| 22 | 29.3 | 15.0 |
| 23 | 29.2 | 15.0 |
| 24 | 17.7 | 14.9 |
| 25 | 27.5 | 13.4 |
| 26 | 19.4 | 13.4 |
| 27 | 14.6 | 13.0 |
| 28 | 28.5 | 12.0 |

C. Thermogravimetric Analysis:

Thermal gravimetric analysis of Compound I aspartame cocrystal Form A was measured using the TA Instruments Q5500 Discovery Series. The TGA thermogram (FIG. 31) shows weight loss of ˜10% from ambient temperature to ˜144° C.

D. Differential Scanning Calorimetry Analysis:

The melting point of Compound I aspartame cocrystal Form A was measured using the TA Instruments Q2500 Discovery Series. The thermogram (FIG. 32) shows an endotherm at ˜147° C.

### 7. Compound I Glutaric Acid Cocrystal Form A

A. Synthetic Procedure:

Form A (˜20.5 mg) and glutaric acid (˜8.2 mg) were combined in a 1-dram vial; approx. 0.3 mL of 7:3 butyl acetate/toluene was added. The mixture was magnetically stirred at room temperature. A thick suspension resulted upon stirring and the solvent mixture was added to maintain a fluid slurry as follows: 0.2 mL (day 1), 0.1 mL (day 2), 0.2 mL (day 3). After one week, the solid material was separated by centrifugation, the remaining liquid was removed via pipette. The sample was dried in a vacuum desiccator for 2-3 hrs.

B. X-Ray Powder Diffraction:

The XRPD diffractogram of Compound I glutaric acid cocrystal Form A (FIG. 33) was acquired at room temperature using Rigaku Smart-Lab X-ray diffraction system. This system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

TABLE 21
XRPD diffractogram of Compound I glutaric acid cocrystal Form A

| XRPD Peaks | Angle (Degrees 2-Theta ± 0.2) | Intensity % |
|---|---|---|
| 1 | 18.9 | 100.0 |
| 2 | 19.1 | 49.7 |
| 3 | 9.4 | 44.9 |
| 4 | 26.9 | 34.2 |
| 5 | 22.2 | 32.9 |
| 6 | 23.2 | 27.5 |
| 7 | 21.9 | 20.4 |
| 8 | 18.0 | 19.5 |
| 9 | 13.5 | 17.9 |
| 10 | 11.0 | 17.5 |
| 11 | 21.1 | 16.3 |
| 12 | 20.4 | 16.2 |
| 13 | 24.9 | 15.1 |
| 14 | 35.9 | 14.5 |
| 15 | 17.7 | 13.1 |
| 16 | 20.8 | 12.0 |
| 17 | 21.4 | 11.2 |
| 18 | 29.3 | 10.8 |
| 19 | 16.2 | 10.5 |

C. Thermogravimetric analysis:

Thermal gravimetric analysis of Compound I glutaric acid cocrystal Form A was measured using the TA Instruments Q5500 Discovery Series. The TGA thermogram (FIG. 34) shows weight loss of ˜5% from ambient temperature to ˜188° C.

D. Differential Scanning Calorimetry Analysis:

The melting point of Compound I glutaric acid cocrystal Form A was measured using the TA Instruments Q2500 Discovery Series. The thermogram (FIG. 35) shows two endotherms at ˜116° C. and ˜227° C.

### 8. Compound I L-Proline Cocrystal Form A

A. Synthetic Procedure:

Approximately ˜25.0 mg of Compound I form A and ˜15.6 mg of L-proline was weighed and placed in ball mill with ˜10 ul of 1-Pentanol. The material was milled at 100 Hz for 30 minutes.

B. X-Ray Powder Diffraction:

The XRPD diffractogram of Compound I L-proline cocrystal Form A (FIG. 36) was acquired at room temperature using Rigaku Smart-Lab X-ray diffraction system. This system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

TABLE 22
XRPD diffractogram of Compound I L-proline cocrystal Form A

| | Angle (Degrees | |
|---|---|---|
| XRPD Peaks | 2-Theta ± 0.2) | Intensity % |
| 1 | 18.2 | 100.0 |
| 2 | 6.0 | 93.3 |
| 3 | 21.7 | 83.6 |
| 4 | 20.2 | 63.4 |
| 5 | 20.7 | 55.9 |
| 6 | 24.3 | 54.6 |
| 7 | 19.5 | 53.3 |
| 8 | 17.9 | 50.5 |
| 9 | 22.0 | 50.2 |
| 10 | 22.9 | 48.4 |
| 11 | 15.6 | 44.4 |
| 12 | 27.2 | 43.2 |
| 13 | 4.9 | 42.8 |
| 14 | 17.6 | 39.2 |
| 15 | 25.9 | 36.8 |
| 16 | 12.0 | 32.1 |
| 17 | 16.5 | 30.4 |
| 18 | 10.9 | 28.4 |
| 19 | 24.0 | 26.5 |
| 20 | 18.4 | 25.6 |
| 21 | 9.8 | 22.2 |
| 22 | 29.4 | 18.7 |
| 23 | 19.0 | 16.9 |
| 24 | 11.2 | 16.8 |
| 25 | 23.4 | 16.8 |
| 26 | 27.6 | 12.7 |
| 27 | 22.5 | 12.2 |
| 28 | 24.9 | 11.9 |

C. Thermogravimetric Analysis:

Thermal gravimetric analysis of Compound I L-proline cocrystal Form A was measured using the TA Instruments Q5500 Discovery Series. The TGA thermogram (FIG. 37) shows weight loss of ˜6% from ambient temperature to ˜130° C.

D. Differential Scanning Calorimetry Analysis:

The melting point of Compound I L-proline cocrystal Form A was measured using the TA Instruments Q2500 Discovery Series. The thermogram (FIG. 38) shows three endotherms at ˜140° C., ˜221° C. and ˜232° C.

### 9. Compound I L-Proline Cocrystal Form B

A. Synthetic Procedure:

Approximately ˜30.0 mg of Compound I Form A and ˜19 mg of L-proline was weighed and placed in ball mill with ˜10 ul of butyl acetate. The material was milled at 100 Hz for 30 minutes.

B. X-Ray Powder Diffraction:

The XRPD diffractogram of Compound I L-proline cocrystal Form B (FIG. 39A) was acquired at room temperature using Rigaku Smart-Lab X-ray diffraction system. This system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

TABLE 23A
XRPD diffractogram of Compound I L-proline cocrystal Form B

| | Angle (Degrees | |
|---|---|---|
| XRPD Peaks | 2-Theta ± 0.2) | Intensity % |
| 1 | 22.5 | 100.0 |
| 2 | 21.7 | 88.2 |
| 3 | 21.2 | 48.0 |
| 4 | 18.7 | 46.0 |
| 5 | 18.3 | 45.2 |
| 6 | 16.0 | 43.9 |
| 7 | 13.1 | 41.4 |
| 8 | 9.8 | 32.1 |
| 9 | 20.7 | 27.0 |
| 10 | 28.5 | 25.1 |
| 11 | 27.1 | 24.7 |
| 12 | 19.0 | 18.0 |
| 13 | 25.8 | 17.2 |
| 14 | 6.6 | 16.8 |
| 15 | 19.6 | 16.4 |
| 16 | 11.3 | 14.6 |
| 17 | 20.4 | 13.5 |
| 18 | 17.5 | 11.6 |
| 19 | 26.2 | 10.4 |

C. Solid State NMR

The ¹³C CPMAS of the L-proline cocrystal Form B of Compound I (FIG. 39B) was acquired at 275K with 12.5 kHz spinning and using adamantane as a reference. The peaks are listed in Table 23B.

TABLE 23B
¹³C CPMAS of Compound I L-proline cocrystal Form B

| | Chem Shift | |
|---|---|---|
| Peak # | [ppm ± 0.2] | Intensity [rel] |
| 1 | 175.9 | 69.2 |
| 2 | 173.6 | 32.3 |
| 3 | 172.3 | 29.5 |
| 4 | 162.3 | 15.3 |
| 5 | 159.8 | 21.6 |
| 6 | 136.5 | 25.8 |
| 7 | 133.2 | 21.2 |
| 8 | 130.3 | 60.5 |
| 9 | 128.0 | 28.3 |
| 10 | 120.0 | 57.9 |
| 11 | 118.7 | 53.2 |
| 12 | 118.2 | 59.2 |
| 13 | 116.0 | 32.7 |
| 14 | 110.2 | 100.0 |
| 15 | 70.3 | 54.0 |
| 16 | 61.8 | 86.8 |
| 17 | 60.6 | 56.1 |
| 18 | 47.4 | 76.1 |
| 19 | 46.9 | 75.3 |
| 20 | 34.2 | 42.3 |
| 21 | 31.8 | 41.7 |
| 22 | 28.6 | 8.2 |
| 23 | 27.6 | 41.5 |
| 24 | 26.6 | 50.4 |
| 25 | 25.3 | 53.2 |
| 26 | 19.3 | 41.3 |

The ¹⁹F MAS of L-proline cocrystal Form B of Compound I (FIG. 39C) was acquired at 275K with 12.5 kHz spinning and using adamantane as a reference. The peaks are listed in Table 23C.

TABLE 23C
¹⁹F MAS of L-proline cocrystal Form B of Compound I

| | Chem Shift | |
|---|---|---|
| Peak # | [ppm ± 0.2] | Intensity [rel] |
| 1 | −116.9 | 12.5 |

D. Thermogravimetric Analysis:

Thermal gravimetric analysis of Compound I L-proline cocrystal Form B was measured using the TA Instruments Q5500 Discovery Series. The TGA thermogram (FIG. 40) shows weight loss of ˜1.6% from ambient temperature to ˜200° C.

E. Differential Scanning Calorimetry Analysis:

The melting point of Compound I L-proline cocrystal Form B was measured using the TA Instruments Q2500 Discovery Series. The thermogram (FIG. 41) shows two endotherms at ˜220° C. and ˜232° C.

### 10. Compound I Vanillin Cocrystal Form A

A. Synthetic Procedure:

Approximately ˜30.1 mg of Compound I form A and ˜12.8 mg of vanillin was weighed and placed in ball mill with ˜10 ul of 1-Pentanol. The material was milled at 100 Hz for 30 minutes.

B. X-Ray Powder Diffraction:

The XRPD diffractogram of Compound I Vanillin cocrystal Form A (FIG. 42A) was acquired at room temperature using Rigaku Smart-Lab X-ray diffraction system. This system was configured for reflection Bragg-Brentano geometry using a line source X-ray beam. The x-ray source is a Cu Long Fine Focus tube that was operated at 40 kV and 44 mA. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1 °2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40 °2θ using a continuous scan of 6 °2θ per minute with an effective step size of 0.02 °2θ.

TABLE 24A
XRPD diffractogram of Compound I vanillin cocrystal Form A

| | Angle (Degrees | |
|---|---|---|
| XRPD Peaks | 2-Theta ± 0.2) | Intensity % |
| 1 | 21.9 | 100.0 |
| 2 | 21.0 | 92.0 |
| 3 | 15.6 | 90.0 |
| 4 | 24.5 | 67.2 |
| 5 | 9.6 | 62.3 |
| 6 | 26.2 | 60.8 |
| 7 | 23.7 | 52.7 |
| 8 | 27.4 | 41.6 |
| 9 | 26.7 | 40.2 |
| 10 | 14.3 | 40.1 |
| 11 | 12.0 | 35.4 |
| 12 | 9.3 | 34.0 |
| 13 | 13.1 | 33.9 |
| 14 | 3.3 | 33.8 |
| 15 | 19.9 | 31.3 |
| 16 | 12.7 | 27.4 |
| 17 | 19.6 | 26.4 |
| 18 | 27.9 | 25.3 |
| 19 | 10.3 | 24.9 |
| 20 | 18.8 | 23.5 |
| 21 | 28.5 | 20.8 |
| 22 | 22.1 | 20.1 |
| 23 | 18.2 | 12.7 |
| 24 | 29.9 | 12.0 |
| 25 | 22.4 | 11.1 |
| 26 | 27.0 | 10.2 |

C. Solid State NMR

The ¹³C CPMAS of the vanillin cocrystal Form A of Compound I (FIG. 42B) was acquired at 275K with 12.5 kHz spinning and using adamantane as a reference. The peaks are listed in Table 24B.

TABLE 24B
¹³C CPMAS of Compound I vanillin cocrystal Form A

| Peak # | Chem Shift [ppm ± 0.2] | Intensity [rel] |
|---|---|---|
| 1 | 191.4 | 42.4 |
| 2 | 175.4 | 25.9 |
| 3 | 171.9 | 24.3 |
| 4 | 163.9 | 8.1 |
| 5 | 161.4 | 11.9 |
| 6 | 153.7 | 51.6 |
| 7 | 147.4 | 51.2 |
| 8 | 135.4 | 23.8 |
| 9 | 132.9 | 20.5 |
| 10 | 130.6 | 48.6 |
| 11 | 129.4 | 100.0 |
| 12 | 129.1 | 65.5 |
| 13 | 128.8 | 50.2 |
| 14 | 127.8 | 57.3 |
| 15 | 121.9 | 42.2 |
| 16 | 120.5 | 33.6 |
| 17 | 119.2 | 49.8 |
| 18 | 116.1 | 66.3 |
| 19 | 114.6 | 50.0 |
| 20 | 113.0 | 43.6 |
| 21 | 110.7 | 35.6 |
| 22 | 107.8 | 39.7 |
| 23 | 74.3 | 49.1 |
| 24 | 59.8 | 16.8 |
| 25 | 59.4 | 23.3 |
| 26 | 54.6 | 61.9 |
| 27 | 44.9 | 20.3 |
| 28 | 44.5 | 27.7 |
| 29 | 35.5 | 35.1 |
| 30 | 18.2 | 38.5 |

The ¹⁹F MAS of vanillin cocrystal Form A of Compound I (FIG. 42C) was acquired at 275K with 12.5 kHz spinning and using adamantane as a reference. The peaks are listed in Table 24C.

TABLE 24C
¹⁹F MAS of vanillin cocrystal Form A of Compound I

| Peak # | Chem Shift [ppm ± 0.2] | Intensity [rel] |
|---|---|---|
| 1 | −115.2 | 12.5 |

D. Thermogravimetric Analysis:

Thermal gravimetric analysis of Compound I vanillin cocrystal Form A was measured using the TA Instruments Q5500 Discovery Series. The TGA thermogram (FIG. 43) shows weight loss of ˜25% from ambient temperature to ˜200° C.

E. Differential Scanning Calorimetry Analysis:

The melting point of Compound I vanillin cocrystal Form A was measured using the TA Instruments Q2500 Discovery Series. The thermogram (FIG. 44) shows an endotherm at ˜136° C.

### 11. 2-Pyridone Cocrystal Form A of Compound I

A. Synthetic Procedure:

Compound I 2-pyridone co-crystal Form A was produced via solvent assisted ball milling. Approximately ˜100 mg of Compound I Form A and ˜25 mg 2-pyridone was weighed and transfer to the ball milling vessel. ˜20 μL 1-pentanol was added to the vessel. The mixture was ball milled for 30 minutes. The solid obtained from this process was a mixture of Compound I 2-pyridone cocrystal Form A, Compound I Form B, and amorphous Compound I.

B. X-Ray Powder Diffraction:

The XRPD diffractogram of 2-pyridone Cocrystal Form A of Compound I (FIG. 45) was acquired at room temperature in transmission mode using a PANalytical Empyrean system equipped with a sealed tube source and a PIXcel 1D Medipix-3 detector (Malvern PANalytical Inc, Westborough, Mass.). The X-Ray generator operated at a voltage of 45 kV and a current of 40 mA with copper radiation (1.54060 Å). The powder sample was placed on a 96 well sample holder with mylar film and loaded into the instrument. The sample was scanned over the range of about 3° to about 40 °2θ with a step size of 0.0131303° and 49 s per step.

TABLE 25
XRPD diffractogram of 2-pyridone coerystal Form A of Compound I

| XRPD Peaks | Angle (Degrees 2-Theta ± 0.2) | Intensity % |
|---|---|---|
| 1 | 19.5 | 100 |
| 2 | 7.2 | 91.7 |
| 3 | 20.5 | 64.9 |
| 4 | 21.2 | 57.6 |
| 5 | 18.9 | 51.9 |
| 6 | 13.2 | 50.5 |
| 7 | 9.3 | 41.2 |
| 8 | 23.4 | 26.2 |
| 9 | 14.4 | 23.9 |
| 10 | 18.4 | 23.7 |
| 11 | 4.7 | 23.2 |
| 12 | 26.0 | 19.9 |
| 13 | 24.7 | 17.4 |
| 14 | 16.9 | 15.4 |
| 15 | 15.8 | 12.9 |

C. Solid State NMR

The ¹³C CPMAS of the 2-pyridone cocrystal Form A of Compound I was acquired at 275K with 120.0 kHz spinning and using adamantane as a reference. FIG. 46A shows full spectrum ¹³C CPMAS and FIG. 46B shows the ¹³C CPMAS after Form B and amorphous subtraction.

TABLE 26
¹³C CPMAS of Compound I 2-pyridone cocrystal Form A

| | Chem Shift | |
|---|---|---|
| Peak # | [ppm ± 0.2] | Intensity [rel] |
| 1 | 176.8 | 34.4 |
| 2 | 175.0 | 17.6 |
| 3 | 173.5 | 29.4 |
| 4 | 165.3 | 98.0 |
| 5 | 162.8 | 13.5 |
| 6 | 160.3 | 17.3 |
| 7 | 142.3 | 54.4 |
| 8 | 136.1 | 76.4 |
| 9 | 135.2 | 67.0 |
| 10 | 129.7 | 80.6 |
| 11 | 119.8 | 100.0 |
| 12 | 115.2 | 33.9 |
| 13 | 112.8 | 32.8 |
| 14 | 112.0 | 27.5 |
| 15 | 111.2 | 28.6 |
| 16 | 110.1 | 28.2 |
| 17 | 107.8 | 56.7 |
| 18 | 76.2 | 37.0 |
| 19 | 75.3 | 30.7 |
| 20 | 63.4 | 9.2 |
| 21 | 62.7 | 10.9 |
| 22 | 58.5 | 34.4 |
| 23 | 46.4 | 25.9 |
| 24 | 45.8 | 31.6 |
| 25 | 36.6 | 41.9 |
| 26 | 28.8 | 9.8 |
| 27 | 22.8 | 15.8 |
| 28 | 19.6 | 38.2 |
| 29 | 17.9 | 13.0 |
| 30 | 15.4 | 15.4 |

The ¹⁹F MAS of 2-pyridone cocrystal Form A of Compound I was acquired at

275K with 120.0 kHz spinning and using adamantane as a reference. FIG. 47A shows full spectrum ¹⁹F MAS and FIG. 47B shows the ¹⁹F MAS after Form B and amorphous subtraction.

TABLE 27
¹⁹F MAS of 2-pyridone cocrystal Form A of Compound I

| | Chem Shift | |
|---|---|---|
| Peak # | [ppm ± 0.2] | Intensity [rel] |
| 1 | −112.1 | 9.8 |
| 2 | −115.5 | 12.5 |

D. Thermogravimetric Analysis:

Thermal gravimetric analysis of Compound I 2-pyridone cocrystal Form A was measured using the TA Instruments Discovery TGA. The thermogram (FIG. 48) shows ˜25% weight loss from ambient to 200° C. with continued weight loss until 300° C.

E. Differential Scanning Calorimetry Analysis:

Differential scanning calorimetry analysis of Compound I 2-pyridone cocrystal Form A was measured using the TA Instruments Q2000 DSC. The thermogram (FIG. 49) shows three endotherms at 102° C., 123° C., and 216° C.

### Other Embodiments

This disclosure provides merely exemplary embodiments of the disclosure. One skilled in the art will readily recognize from the disclosure and claims, that various changes, modifications and variations can be made therein without departing from the spirit and scope of the disclosure as defined in the following claims.

1. A crystalline form of Compound I: 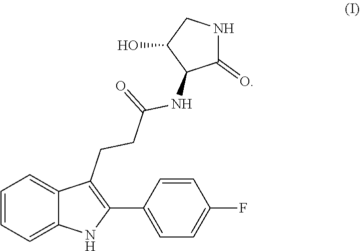 [SMILES: O=C(CCc1c(-c2ccc(F)cc2)[nH]c2ccccc12)N[C@@H]1C(=O)NC[C@H]1O]
selected from: Form B, citric acid cocrystal Form A, piperazine cocrystal Form A, urea cocrystal Form A, nicotinamide cocrystal Form A, nicotinamide cocrystal Form B, aspartame cocrystal Form A, glutaric acid cocrystal Form A, L-proline cocrystal Form A, L-proline cocrystal Form B, vanillin cocrystal Form A, and 2-pyridone cocrystal Form A.

2. A method of preparing a crystalline form of Compound I comprising:
a) mixing Compound I with n-pentanol at 65° C., and
stirring said mixture at 65° C. for at least 2 hours,
and isolating Compound I Form B;
b) mixing Compound I Form A with citric acid,
dissolving the mixture in 2 butanone (MEK),
stirring for 30 min-1 hour to form a slurry,
centrifuging and then drying the solid at 55° C. overnight with nitrogen bleed,
isolating Compound I citric acid Form A;
c) mixing Compound I Form A with piperazine and ethyl acetate,
sonicating mixture for about 30 minutes at ambient temperature,
isolating Compound I piperazine cocrystal Form A;
d) dissolving Compound I Form A in solvent and adding urea,
stirring for 1 hour at ambient temperature to form pre-saturated solution,
adding a preground mixture of Compound I Form A and dry urea to make a slurry,
heating to 25° C. and stirring for about 24 hours, and
isolating Compound I urea cocrystal Form A;
e) dissolving Compound I Form A in solvent and adding nicotinamide,
stirring for 1 hour at ambient temperature to form pre-saturated solution,
adding a preground mixture of Compound I Form A and dry nicotinamide to make a slurry,
heating to 25° C. and stirring for about 24 hours, and
isolating Compound I nicotinamide cocrystal Form A;
f) mixing Compound I Form A with nicotinamide (1:1) in a ball mill vessel with pentanol,
shaking at 15 hertz for about 30 minutes, and
isolating nicotinamide cocrystal Form B of Compound I;
g) mixing Compound I Form A with aspartame in a ball mill vessel with pentanol,
shaking at 100 hertz for about 30 minutes,
isolating aspartame cocrystal Form A of Compound I;
h) combining Compound I Form A and glutaric acid with butyl acetate/toluene,
stirring magnetically at room temperature and adding butyl acetate/toluene to maintain a fluid slurry,
centrifuging after about one week and removing remaining fluid,
drying solid in vacuum dessicator for 2-3 hours to provide glutaric acid cocrystal Form A of Compound I;
i) mixing Compound I Form A with L-proline in a ball mill with pentanol,
milling at 100 hertz for about 30 minutes,
isolating L-proline cocrystal Form A of Compound I;
j) mixing Compound I Form A with L-proline in a ball mill with butyl acetate,
milling at 100 hertz for about 30 minutes,
isolating L-proline cocrystal Form B of Compound I;
k) mixing Compound I Form A with vanillin in a ball mill with pentanol,
milling at 100 hertz for about 30 minutes,
isolating vanillin cocrystal Form A of Compound I;
l) mixing Compound I Form A with 2-pyridone in a ball mill with pentanol,
milling at 100 hertz for about 30 minutes,
isolating 2-pyridone cocrystal Form A of Compound I.

3. A pharmaceutical composition comprising a solid form of Compound I according to claim 1.

4. A pharmaceutical composition comprising a solid form of Compound I prepared by a method according to claim 2.

5. A method of treating APOL1 mediated kidney disease comprising administering to a patient in need thereof a solid form of Compound I according to claim 1 or a pharmaceutical composition according to claim 3 or claim 4.

6. A method of inhibiting APOL1 activity comprising contacting said APOL1 with a solid form of Compound I according to claim 1 or a pharmaceutical composition according to claim 3 or claim 4.
